(19) United States
(12) Patent Application Publication
(10) Pub. No.: US 2019/0144304 A1
(43) Pub. Date: May 16, 2019

(54) SYSTEM AND METHOD FOR COMPREHENSIVE TREATMENT OF CULTIVATION POLLUTION IN SCALABLE PIG FARM

(71) Applicants: Shen WANG, CHANGSHA (CN); HUNAN SAKAL ENVIRONMENTAL SCIENCE AND TECHNOLOGY CO., LTD., CHANGSHA (CN)

(72) Inventors: Shen WANG, CHANGSHA (CN); Wen KUANG, CHANGSHA (CN); Juncan WANG, CHANGSHA (CN)

(73) Assignee: HUNAN SAKAL ENVIRONMENTAL SCIENCE AND TECHNOLOGY CO., LTD.

(21) Appl. No.: 16/244,695

(22) Filed: Jan. 10, 2019

Related U.S. Application Data
(63) Continuation of application No. PCT/CN2016/090661, filed on Jul. 20, 2016.

(30) Foreign Application Priority Data
Jul. 11, 2016 (CN) 201610538015.2

(51) Int. Cl. C02F 1/26; A01K 1/01; A01C 3/02; C12M 1/16; C02F 1/66

(52) U.S. Cl. CPC C02F 1/26; A01K 1/0103; C02F 2103/20; C12M 21/16; C02F 1/66; A01C 3/023

(57) ABSTRACT

A system for comprehensive treatment of cultivation pollution in a scalable pig farm includes a source separation pigsty, a pigsty flushing water treatment system, a solid-liquid separation system, a solid high-temperature aerobic fermentation system, a liquid high-temperature aerobic fermentation system, an odor and flue gas treatment system, a boiler system and a test and control system. The source separation pigsty is designed into a pigsty that separates feces and urine from rainwater, pigsty flushing water and residual drinking water. The sludge pump pumps the feces and urine to the solid-liquid separation device. Solids separated by the solid-liquid separation device are conveyed to the solid high-temperature aerobic fermentation system, and liquid separated by the solid-liquid separation device is conveyed to the liquid high-temperature aerobic fermentation system. The boiler system includes a boiler, a circulating pump, a hot water pipeline and a water return pipeline.

### CROSS-REFERENCE TO RELATED APPLICATIONS

This application is a continuation of International Patent Application No. PCT/CN2016/090661 with a filing date of Jul. 20, 2016, designating the United States, now pending, and further claims priority to Chinese Patent Application No. 201610538015.2 with a filing date of Jul. 11, 2016. The content of the aforementioned applications, including any intervening amendments thereto, are incorporated herein by reference.

### TECHNICAL FIELD

The present disclosure belongs to the technical field of environmental protection, and particularly relates to a system and method for comprehensive treatment of cultivation pollution in a scalable pig farm.

### BACKGROUND OF THE PRESENT INVENTION

The intensive and large-scale development of the livestock and poultry breeding provides abundant and high-quality livestock and poultry products for the market, and also, feces enormous environmental pressure caused by a large number of livestock and poultry pollutants. In 2009, the livestock and poultry feces emissions were 3.264 billion tons by fresh weight in China, which was 1.6 times the total industrial solid waste emissions during the same period (Statistics Bureau of she People's Republic of China, 2010). Firstly, livestock and poultry feces will bring serious odor and pollute air. The livestock and poultry feces will be decomposed into malodorous gases such as ammonia, sulfuric acid, vinyl alcohol, dimethyl sulfide, hydrogen sulfide, methylamine and trimethylamine in the case of poor ventilation and has rotten onion odor, slinky egg odor, fish odor and the like. These gases are harmful to human health and aggravate the air pollution. Secondly, the livestock and poultry feces will pollute water. Random discharge of the livestock and poultry feces easily leads to eutrophication of water, which will worsen the water quality. In filtration of the feces and sewage into the ground may also cause an extremely high content of nitrate in groundwater. Thirdly, the livestock and poultry feces will spread zoonotic diseases. There are more than 90 kinds of zoonotic diseases transmitted from animals to humans. Carriers of these zoonotic diseases are mainly livestock and poultry feces and excretions, if these livestock and poultry feces are not properly treated and rationally used, they will not only cause huge negative impact on the ecological environment and seriously affect the health of people, but also restrict the sound development of the livestock and poultry breeding industry.

At present, feces cleaning modes commonly used in large-scale pig farms in China and abroad mainly include the processes of water submerging, rinsing and dry collection. The mode of feces cleaning by water submerging is often used at present, but it has the defects that because the feces stay in a pigsty for a long time, part of the feces will be anaerobically fermented and will produce a large amount of harmful gases such as ammonia, hydrogen sulfide and methane in a feces pit, which would reduce the appetite of pigs and harm the health of the pigs. People had to use high energy consumption forced ventilation to solve the problem of poisonous gas pollution. However, in winter, the forced ventilation makes it impossible to keep warm in the pigsty, and the pigs are susceptible to infectious diseases such as influenza, and meanwhile, a large amount of poisonous and harmful gases also affect the work of breeders and endanger the health of the breeders. The mode of feces cleaning by rinsing is also a common feces cleaning mode, bur it has the defects of high water consumption and severe waste of water resources; and in the later feces treatment process, after solid and liquid are separated, the nutrient content in dry substances is low, which lowers the fertilizer value, and a large amount of sewage is produced, and the content of most soluble organic matters in the sewage is still high, thereby increasing the treatment difficulty, in the dry collection mode, the feces are shunted as soon as it is produced. Dry feces are mechanically or manually collected, swept and transported, and urine and pigsty flushing water flow out from a sewer and are separately heated. The dry collection process includes manual feces cleaning and mechanical feces cleaning. The manual feces cleaning has the shortcomings of cross infection easily caused between human and livestock, high labor intensity, poor working environment and low productivity. The mechanical feces cleaning has the advantages of relieving the labor intensity saving labors and improving the working efficiency, but feces cleaning equipment manufactured in China at present are low in use reliability complicated, relatively high in fault occurrence rate and difficult in maintenance. Whether in the manual feces cleaning mode or in the mechanical feces cleaning mode, the urine will be mixed with the pigsty flushing water and residual pig drinking water, so that pollutants in the pigsty flushing water have high concentration, and the treatment volume and she treatment difficulty of the pigsty flushing water are increased.

In summary, the above feces cleaning modes mainly have the following problems that: (1) in the absence of an independent collection and conveying system for the residual pig drinking water, the residual pig drinking water is directly mixed with, the feces, the urine and the pigsty flushing water, thereby increasing the pollution volume and the treatment cost. According to the statistics, water leaked by the pigs that drink water in a duckbill type chinking bowl accounts for about 20%-40% of the total amount of the pigsty flushing water; and (2) the pigsty flushing water is not separated from the feces and the urine, but is directly mixed with the feces and the urine for subsequent treatment. The treatment method generally includes: performing anaerobic fermentation on the mixture in a biogas generating pit, then separating solid from liquid, manufacturing a solid organic fertilizer by a solid part obtained by separation, and treating a liquid part as sewage for “up-to-standard discharge”. The method has the disadvantage that, lots of nutrients are dissolved in the liquid, so that the produced solid organic fertilizer has low content of nutrients and low quality. The liquid is rich in organic matters and nitrogen phosphorus potassium, and COD (chemical oxygen demand) and the ammonia nitrogen concentrations are high, so that it is very difficult for a common sewage treatment technology to treat the liquid to meet the discharge standard. Even in accordance with limit values, i.e. COD 400 mg/L, ammonia nitrogen 80 mg/L and total phosphorus 8 mg/L. in the current Discharge Standard of Pollutants for Livestock and Poultry Breeding (GB 18596-2001), which are respectively 8 times, 16 times and 16 times the limit values in the Discharge Standard of Pollutants for Municipal Wastewater Treatment Plant (GB18918-2002, A level of first class), the so-called “up-to-standard discharge” is actually the source that causes water eutrophication, and the “up-to-standard discharged” sewage, continuously pollutants the environment.

In aerobic fermentation (composting), organic matters maybe degraded through microorganisms to realize reducing, harmlessness and recycling treatment for organic waste. Afield composting fermentation technology widely used at present mainly has a series of problems of huge occupation area, long fermentation time (generally, it takes about 15-30 days for primary fermentation), reduction and even stop of the fermentation speed in low-temperature weather, difficulty in collection and treatment of odor which pollutes the environment and the like. Trough composting is mostly researched at present, and aims to improve the efficiency or effect of aerobic composting through ventilation and forced oxygen feeding, turning or stirring and the like. This mode has the problems of high investment cost for infrastructures and turning equipment and severe secondary pollution caused by heat and odor which are produced by fermentation and generally allowed to be discharged into the atmosphere. A famous Dano drum type aerobic reactor has the features of high fermentation efficiency, small occupation area and the like relative to site fermentation, but a fermentation effect, depends on factors such as the length of a drum. To prolong the retention time of fermentation raw material in the drum reactor to guarantee the fermentation effect, the length of the drum is generally designed to be 10 times and even 20 times or more the diameter of the drum, so that the occupation area is still very large, and the equipment manufacturing cost is high. Meanwhile, the aerobic reactor also has the defects of extremely small contact area between newly added fermentation raw material (organic waste) and the fermented raw material, relatively low reaction speed caused by insufficiency of zymophyte of the newly added fermentation raw material and the like,

At present, the mortality rate of pigs in pig farms is generally 3-5%. and the proportion will be greater in case of epidemic situations. Pollution to a drinking water source is often caused by improper treatment, and even unscrupulous vendors resell dead pigs to make exorbitant profits, which directly threatens the food safety of ordinary people. The problem on isolation and treatment of carcasses of these dead animals that carry harmful bacteria is needed to be solved urgently. At present, harmless treatment methods of the dead pigs mainly include high-temperature incineration, landfill, high-temperature composting and the like. The high-temperature incineration mode has extremely high requirements for equipment and is relatively high in one-time investment cost, and most large-scale farms are not qualified for high-temperature incineration. Furthermore, centralized incineration has a series of problems of emission pollution, complicated collection and transferring processes, insufficient supervision measures as id the like. In addition, an extremely severe secondary pollution problem will be caused in the treatment process. The landfill mode is widely used at present, but it has the defects that the transportation and landfill costs are high, the subsidy for the dead pigs is much less than the landfill cost, and most farm staff do not landfill the waste sanitarily due to lack of health and safety knowledge, thereby leading to severe secondary pollution. The high-temperature composing is also one of the harmless treatment methods of the dead pigs, but has the defects that mechanical loss, energy consumption and equipment cleaning in a pretreatment process such as mincing of the dead pigs may increase the treatment cost; operators ate prone to have cross infection in processes such as mincing of the dead pigs, equipment clearing and replacement of easily damaged parts thereby increasing the risk of epidemic transmission; and composting occupies a large area, takes long time and is easily affected by weather conditions. Because the dead pigs carry lots of pathogenic microorganisms, it is hard for traditional fermentation modes to thoroughly kill these pathogenic microorganisms. A large number of pollutants such as placentas are produced in a pig breeding farm in a piglet breeding process. If these pollutants which are rich in nutrition and very easy to rot are not treated in time, it is very easy to cause pollution. The landfill treatment mode is often used at present and has the same problems as those in treatment of the dead pigs.

The pollutants of the large-scale pig farm not only include the pig feces and the pigsty flushing water but also include the dead pigs, the placentas, and odor and flue gas which are produced in the treatment process. There is no proper method for simultaneously treating the above pollutants at present. A Chinese patent CN203568937 discloses a livestock and poultry feces treatment system, including a rain-sewage separation system, a dry-wet separation system, a solid-liquid separation system and an ecological purification system. This system has the problems that because the feces are mixed with the pigsty flushing water and the residual drinking water and then are subjected to solid-liquid separation, the liquid obtained after the solid-liquid separation contains a large number of organic matters and nutrients, and the contents of COD and ammonia nitrogen are obviously increased, leading to waste of resources, increase of the treatment capacity and difficulty of subsequent sewage treatment and increase of the treatment cost. Separated solid materials are fermented by a natural composting mode. This natural composting mode occupies a large site area, is low in fermentation efficiency, causes pool organic fertilizer qualify, is easily affected by environmental factors and easily causes secondary pollution. In addition, the system does not involve treatment of the carcasses of dead livestock and poultry, the placentas and the like in livestock and poultry farms.

A Chinese patent CN201520168695.4 discloses a closed type biodegradable harmless treatment device for dead livestock and poultry. In this patent, the carcasses of the dead livestock and poultry are degraded by adding auxiliary materials such as biodegradable dead body decomposer; and parasites, eggs, pathogenic bacteria and viruses are killed through anaerobic fermentation. The device has the disadvantages that the decomposing efficiency of the carcasses of the dead livestock and poultry in a solid state is much lower than that under a liquid condition, and the anaerobic fermentation may not completely kill the harmful pathogenic bacteria in the carcasses of the dead livestock and poultry, thereby causing the risk of secondary pollution

A patent CN102964149 discloses a pollution treatment method of livestock and poultry farms, which does not involve the disposal of the carcasses of the dead livestock and poultry, the placentas and the like and the use of hot flue gas and fermentation heat which are produced by boiler combustion. Furthermore, the pigsty flushing water is treated by an SBR (Sequencing Batch Reactor), so that it is very difficult for a large discharge amount of the pigsty flushing water to meet the standard in the peak season of slaughtering.

### SUMMARY OF PRESENT INVENTION

The purpose of the present disclosure is to solve the problems in the above background, so as to provide a system and method for comprehensive treatment of cultivation pollution in a scalable pig farm. Pig feces urine, pigsty flushing water, residual drinking water carcasses of dead pigs, placentas, odor and flue gas are respectively treated from their sources. The pig feces, the urine, the dead pigs and the placentas are recycled, and heat in the odor and the flue gas is comprehensively used so as to comprehensively treat the pollution in the large-scale pig farm and really realize “zero emission, zero pollution and recycling use” of pollutants in the large-scale pig farm.

The present disclosure adopts the following technical solution:

A system for comprehensive treatment of cultivation pollution in a scalable pig farm includes a source separation pigsty, a pigsty flushing water treatment system, a solid-liquid separation system a solid high-temperature aerobic fermentation system, a liquid high-temperature aerobic fermentation system, an odor and flue gas treatment system, a boiler system and a test and control system. The source separation pigsty is designed into a pigsty that separates feces and urine from rainwater, pigsty flushing water and residual drinking water. The rainwater and the residual drinking water are discharged into an outdoor drainage ditch. The feces and urine are conveyed to a feces collection pit of the solid-liquid separation system. The pigsty flushing water is discharged into a pigsty flushing water pit of the pigsty flushing water treatment system. The solid-liquid separation system is composed of a sludge pump installed at the bottom of the feces collection pit, a solid-liquid separation device and conveying equipment. The sludge pump pumps the feces and urine to the solid-liquid separation device. Solids separated by the solid-liquid separation device are conveyed to a feed port of the solid high-temperature aerobic fermentation system, and liquid separated by the solid-liquid separation device is conveyed to a liquid inlet of the liquid high-temperature aerobic fermentation system. Fermentation odor exhaust ports of the solid high-temperature aerobic fermentation system and the liquid high-temperature aerobic fermentation system and a flue gas exhaust port of the boiler system are connected with an odor and flue gas treatment system through pipelines. The boiler system includes a boiler, a circulating pump, a hot water pipeline and a water return pipeline. The hot water pipeline of the boiler is connected with a heat exchange jacket or coil pipe of the solid high-temperature aerobic fermentation system and a jacket or coil pipe of the liquid high-temperature aerobic fermentation system. The circulating pump is installed in the water return pipeline. All sensors of the test and control system are disposed in the above systems to detect all key parameters. The test and control system controls connection of the above systems.

In the above technical solution, the source separation pigsty is designed into a device and system for rainwater and sewage separation, drinking water and sewage separation and separation of feces and urine from pigsty flushing water. The rainwater is blocked by the pigsty and discharged through an external ditch, so as to prevent the rainwater from being mixed into the feces and urine. The residual drinking water is discharged to the external ditch in time, so as to prevent the residual drinking water from being mixed into the feces and urine. The pigsty flushing water is not mixed into the feces and urine. During pigsty flushing, the feces and urine are firstly discharged completely, and then the pigsty is flushed, so that the pigsty flushing water is collected into the pigsty flushing water pit, and the feces and urine liquid is collected into the feces collection pit.

In the above technical solution, the pigsty flushing water treatment system is composed of the pigsty flushing water pit, an ABR (anaerobic baffled reactor) and a plurality of parallel-connected SBRs. A water overflow port is formed in the upper part of the pigsty flushing water pit. A large grating and a small grating are respectively installed on the water inlet outer side and the water outlet inner side of the pigsty flushing water pit. The water outlet side of the overflow port is connected with a water inlet of the ABR through an overflow pipe. A water outlet of the ABR is connected with water inlets of the parallel-connected SBRs through pipelines respectively. An electromagnetic valve is installed in front of the water inlet of each SBR. The water outlet of each SBR is connected to an ecological wetland through a pipeline. Settled sludge in the pigsty flushing water pit, the ABR and the SBRs are conveyed to a feed port of the solid high-temperature aerobic fermentation reactor and then are mixed with feces for fermentation to prepare a solid organic fertilizer.

In the above technical solution, when the source separation pigsty is flushed, the pigsty flushing water enters the pigsty flushing water pit through the coarse grating and flows to the ABR through the fine grating and the overflow pipe. The pigsty flushing water is subjected to sludge settling and anaerobic fermentation of the ABR, and then fermentation liquid enters the first SBR. After the liquid level of the first SBR reaches a designed liquid level of the SBR, the test and control system controls the electromagnetic valves in front of the SBRs to turn off the electromagnetic valve in front of the first SBR and tarn on the electromagnetic valve of the second SBR, so as to allow the liquid levels of ail the SBRs to reach the designed liquid levels. The test and control system realizes an aerobic-anaerobic alternating technical process on all SBRs according to an SBR technique by controlling intermittent aeration. After the SBRs complete a complete SBR treatment technique, supernatant is conveyed to the ecological wetland through the conveying equipment and then discharged.

In the above technical solution, the solid high-temperature aerobic fermentation system includes 1 to X solid high-temperature aerobic fermentation reactors, wherein X is more than or equal to 1. Each solid high-temperature aerobic fermentation reactor is composed of an inclined horizontal drum, a feed side sealing cover labyrinth sealing device, a discharge side sealing cover labyrinth sealing device, a power supporting wheel group, a stirring and anti-sticking device and an integrated base. A water jacket is arranged outside the horizontal drum. The feed side is higher than the discharge side. The horizontal drum, a feed side sealing cover, a discharge side sealing cover and the labyrinth sealing devices on both sides form a closed fermentation space. A feed hole and an exhaust hole are formed in the upper part of the feed side sealing cover. An air inlet hole is formed in the upper part of the discharge side sealing cover. A discharge hole is formed in the lower part of the discharge side sealing cover. A discharge gate is installed on the discharge hole. The stirring and anti-sticking device is positioned in the horizontal drum which is disposed on the power supporting wheel group. The power supporting wheel group, the feed side sealing cover and the discharge side sealing cover are fixed to the inclined integrated base to form a whole.

In the above technical solution, the water jacket outside the horizontal drum is divided into several parts by a rolling ring. The several parts are connected into a whole through a water jacket connection pipe. The water jacket is led to the axis of a horizontal drum sealing covet through a water jacket extraction pipe and is connected with an external circulating water pipe through a rotating joint installed at the axis of the sealing cover.

In the above technical solution, a heat preservation layer covers the outer surface of the water jacket arranged outside the horizontal drum and is made of heat preservation and isolation material.

In the above technical solution, the power supporting wheel group is mainly composed of a supporting wheel group, a power driving device and the like. Power driving adopts multi-wheel driving. The structure of the power driving device is that: a motor, a speed reducer, a shaft coupler or a motor, a speed reducer and a chain transmission device or a belt transmission device which are connected with each supporting wheel in sequence for connection transmission in sequence, so that each supporting wheel is a driving wheel. The supporting wheels are controlled to cooperate with one another to drive the horizontal drum to rotate. At least two groups of power supporting wheel groups, and the number of the power supporting wheel groups is determined according to the length of the drum.

In the above technical solution, the structure and the principle of the feed side sealing cover labyrinth sealing device are completely the same as those of the discharge side sealing cover labyrinth sealing device, and each of the structures is that two or more concentric cylindrical hoods having unequal diameters are perpendicularly welded and fixed to the inner side of the sealing cover, and the cylindrical hoods are consistent in height. Correspondingly, a radial lining ring is welded and fixed to the inner wall of the drum at an end part of the horizontal drum. The periphery of the lining ring is hermetically welded and fixed with the inner wall of the drum. One, two or more concentric cylindrical bodies having unequal diameters are perpendicularly welded on the lining ring, and the concentric cylindrical bodies are consistent in height. Moreover, the height of each of the concentric cylindrical bodies is equal to that of each of the concentric cylindrical hoods on the sealing cover. The concentric cylindrical hoods on the inner sides of the sealing covers and the concentric cylindrical bodies on the lining ring at the end part of the horizontal drum are alternately sheathed and sealed in a labyrinth manner. The labyrinth scaling effect is guaranteed by gaps between the inner sides of the sealing covers and the end surface of the horizontal drum. If the gaps between the inner sides of the sealing covers and the end surfaces of the horizontal drum are smaller, fewer materials are leaked, so that the positions of the end covers on both sides are adjusted to allow the drum to rotate flexibly so as to achieve a sealing effect of least leakage.

The number of the concentric cylindrical hoods on the inner side of each of the sealing covers is increased, and correspondingly, the number of the concentric cylindrical bodies on the inner side at an end part of the horizontal drum is increased to increase the number of labyrinth grids, so as to lengthen the labyrinth and reduce the leakage.

In the above technical solution, according to the length of the horizontal drum, the stirring and anti-sticking device may be composed of one or more cage-shaped structures. When the horizontal drum is relatively short, the stirring and anti-sticking device may be composed of only one cage-shaped structure. When the horizontal drum is relatively long, the stirring and anti-sticking device may be composed of a plurality of cage-shaped structures. Each of the cage-shaped structures is composed of two coaxial supporting plates and a plurality of shoveling plates. The supporting plates are circular rings, and both ends of each of the plurality of shoveling plates are respectively connected and fixed with the two coaxial supporting plates. Correspondingly, contact blocks are disposed on the inner wall of the horizontal drum. The plurality of shoveling plates are parallel to the axes of the cage-shaped structures, or the plurality of shoveling plates and the axes of the cage-shaped structures form inclined angles, or the plurality of shoveling plates are of spiral curve shapes. When the horizontal drum rotates, the contact blocks on the inner wall drive the stirring and anti-sticking device to rotate.

In the above technical solution, the movement of a fermentation raw material from the feed side to the discharge side in the horizontal drum may be realized through a certain angle formed between the horizontal drum and a horizontal plane, and also may be realized through spiral shapes formed by certain inclined angles between the shoveling plates of the stirring and anti-sticking device and the axis of the horizontal drum and backward rotation of the horizontal drum.

Because each of the shoveling plates has a certain width, the stirring and anti-sticking device drives materials at the inner bottom of the horizontal drum to move upwards, and the materials are separated from the shoveling plates and thrown away and fall back to the bottom of the horizontal drum under the gravity action, so as to achieve a material throwing effect. When the shoveling plates of the stirring and anti-sticking device and their axes form certain angles or spiral lines, the drum rotates forwards By virtue of the action of the spiral shoveling plates of the cage-shaped structures in the solid high-temperature aerobic fermentation reactors, the forward rotating drum shovels up the materials and conveys the fermentation law materials to the discharge side. During feeding and discharging through the reactors, by virtue of the action of the spiral shoveling plates of the cage-shaped structures in the solid high-temperature aerobic fermentation reactors, the backward rotating drum shovels up the materials and conveys the fermentation raw materials to the feed side so that the fermentation materials may not compact the discharge side sealing cover. Because the cage-shaped structures of the stirring and anti-sticking device collide with different contact blocks in the drum and rotate under the driving of the contact blocks, the cage-shaped structures and the inner wall of the drum may slide relatively, so that the fermentation raw materials may not be adhered to the inner wall of the drum of the solid high-temperature aerobic fermentation reactor.

In the above technical solution, an included angle of 0-5 degrees formed between the integrated base and the horizontal plane is adjustable. The drum slantways lies down by adjusting the included angle, so as to adjust the conveying speed of the fermentation raw material to the discharge end.

In the above technical solution, a stop wheel is also disposed on the integrated base. The stop wheel is connected to the integrated base in a bolting manner. A waist-shaped hole groove is formed in a stop wheel seat. The stop wheel is adjusted through the waist-shaped hole groove so as to come into contact between the stop wheel and the side line of the rolling ring. The stop wheel keeps off an axial component force of the horizontal drum, so as to prevent the drum from moving along the axis.

In the above technical solution, the liquid high-temperature aerobic fermentation system is mainly composed of 1 to N liquid high-temperature aerobic fermentation reactors, wherein N is more than or equal to 1. Each liquid high-temperature aerobic fermentation reactor mainly includes a top cover, a tank body, a lifting device and a hanging basket. A feed port is formed in the top cover, and a discharge port is formed in the bottom of the tank body. A heat exchange coil is arranged in the tank and is connected with an external hot water pipeline through a water inlet flange and a water outlet flange which are installed on the tank body. An aeration device is also arranged in the tank and is connected with an aeration pipeline and a fan through an air inlet flange installed on the tank body. An exhaust port flange is also installed on the top cover of the tank body to exhaust aeration waste gas. The discharge port is connected to a biogas generating pit through a pipeline. The aeration device is connected to an external fan through an air inlet pipeline. The lifting device is used for lifting and transferring the top cover component and the hanging basket. The hanging basket is composed of a hanging basket mam body, a hanging basket door and a lock catch. The hanging basket door may be laterally opened from one side and is used for putting dead livestock or placenta into the hanging basket. Steel meshes are welded at the upper part, the bottom and the side wall of the hanging basket main body. The hanging basket carries the dead livestock and the placentas and puts the dead livestock and the placentas into the feces and urine fermentation liquid for fermentation. If certain farms are qualified for treating the dead livestock and the placentas in an incineration way or other sanitary ways, the hanging basket may be omitted.

In the above technical solution, the tank body and the top cover of the liquid high-temperature aerobic fermentation reactor are both composed of outer shells, heat preservation layers and inner layers. The inner layers are made of an anti-corrosion material, and the heat preservation layers are made of a heat preservation and isolation material.

In the above technical solution raw materials of the liquid high-temperature aerobic fermentation system the mainly from a mixture of feces and urine of piglets in a suckling period and a nursing period and a liquid part separated by the solid-liquid separation device from feces and urine of fattening pigs, boars and sows, and a solid part separated from the feces and urine of the fattening pigs, the boars and the sows is conveyed to the solid high-temperature aerobic fermentation reactor.

In the above technical solution, the odor and flue gas treatment system includes an odor heat exchange condenser, a flue gas heat exchange condenser, a biological deodorization filtering tower, an induced draft fan, a temperature sensor, a three-way electric regulation valve and an electromagnetic valve. Each heat exchange condenser includes an upper end cover, a tank body and a lower end cover which are connected and fixed in sequence. The upper end of the upper end cover is provided with an odor inlet flange. An odor collection pipeline is connected and fixed with the odor inlet flange. The lower part of the side wall of the tank body is provided with a fresh an inlet flange, and the upper part is provided with a hot air exhaust flange. An upper pipe plate is installed at the upper part of the tank body, and a lower pipe plate is installed at the lower part of the tank body. A plurality of holes fire uniformly formed in the upper and the lower pipe plates. A heat exchange pipe passes through the corresponding holes of the upper pipe plate and the lower pipe plate to connect the upper pipe plate with the lower pipe plate. Both ends of the heat exchange pipe are respectively fixed to the upper and the lower pipe plates, so that a closed cavity is formed among the upper and the lower pipe plates, the outer side of the heat exchange pipe and the outer wall of the tank body and is communicated with the outside through the fresh air inlet flange and the hot air exhaust flange. A plurality of pull rods are uniformly fixed to the lower pipe plate. A plurality of partition plates are uniformly arranged in a space between the fresh air inlet flange and the hot air exhaust flange in the tank body. The partition plates are fixed to the pull rods. An inner cavity of the heat exchange pipe is communicated with the upper end cover and the lower end cover. A U-shaped pipe is arranged at the bottom of a lower cover plate. An odor exhaust flange is arranged on the side wall of the lower end cover.

In the above technical solution, the volume of the lower end cover of each heat exchange condenser is greater than or equal to that of the upper end cover. Each partition plate is of a trimmed circular structure and has a diameter equal to the inner diameter of the tank body. The partition plates are uniformly distributed in the tank body in a disordered manner along the axial direction. The partition plates are fixed to the pull rods, so that fresh air flows in a “Z”-shaped manner so as to add air paths and enlarge contact areas of the air and the heat exchange pipes.

In the above technical solution, a lower end cover water outlet is positioned at the lowest position of the lower end cover and is connected with a water inlet of the U-shaped pipe. The highest, liquid level of the U-shaped pipe is lower than the lowest point of the odor exhaust flange.

In the above technical solution, the odor and flue gas treatment system includes the following treatment systems:

(1) an odor treatment system of the solid high-temperature aerobic fermentation system, wherein the exhaust port of the solid high-temperature aerobic fermentation reactor is connected with a heat exchange air inlet of the odor heat exchange condenser A; a heat exchange exhaust port of the odor heat exchange condenser A is connected with the input end of the induced draft fan; an electromagnetic valve and a bypass branch are arranged on the air inlet pipeline of the odor heat exchange condenser A; the bypass branch of the odor heat exchange condenser A is provided with an electromagnetic valve; the output end of the induced draft fan is connected with an air inlet of the biological deodorization filtering tower, and a temperature sensor is installed on a main-path air inlet pipeline of the biological deodorization filtering tower; a biological deodorization filler is arranged in the biological deodorization filtering tower; the air inlet of the odor heat exchange condenser A is connected with the atmosphere, and an air output port is connected to the air inlet of the solid high-temperature aerobic fermentation reactor through a pipeline; and

(2) an odor treatment system of the high-temperature aerobic fermentation system and a flue gas treatment system of a boiler system, wherein the flue gas exhaust port of a boiler is connected with one air inlet input end of a three-way electric regulation valve, and the other air inlet input end of the three-way electric regulation valve is connected with the atmosphere; the output end of the three-way electric regulation valve is connected with the input end of an aeration fan; the output end of the aeration fan is connected with an air inlet flange of the liquid high-temperature aerobic fermentation reactor; an exhaust flange of the liquid high-temperature aerobic fermentation reactor is connected with a heat exchange air inlet of the odor heat exchange condenser B; a heat exchange exhaust port of the odor heat exchange condenser B is connected with the input end of the induced draft fan; an electromagnetic valve and a bypass branch are arranged on an air inlet pipeline of the odor heat exchange condenser B; the bypass branch of the odor heat exchange condenser B is provided with an electromagnetic valve; the output end of the induced draft fan is connected with an air inlet of a biological deodorization filtering tower, and a temperature sensor is installed on a main-path air inlet pipeline of the biological deodorization filtering tower; a biological deodorization filler is arranged in the biological deodorization filtering tower; an air inlet of the odor heat exchange condenser B is connected with the atmosphere; and an air output port is connected to the air inlet of the boiler through a pipeline.

In the above technical solution, a treatment method of the odor and flue gas treatment system includes:

(1) an odor treatment method of the solid high-temperature aerobic fermentation system: odor exhausted by the solid high-temperature aerobic fermentation reactor through the exhaust port is cooled by the odor heat exchange condenser A, then is absorbed and converted through the biological deodorization filtering tower and is discharged after reaching the standard; hot air subjected to heat exchange through the odor heat exchange condenser A enters the solid high-temperature aerobic fermentation reactor through the air inlet of the solid high-temperature aerobic fermentation reactor to provide fresh hot air for the solid high-temperature aerobic fermentation reactor; and

(2) an odor treatment method of the high-temperature aerobic fermentation system and a flue gas treatment method of the boiler system: the aeration fan adjusts the opening of the three-way electric regulation valve according to an oxygen demand of a material in the liquid high-temperature aerobic fermentation reactor to aerate the liquid high-temperature aerobic fermentation reactor, so that the air input ends of a hearth of the boiler and the three-way electric regulation valve are in negative pressure states all the time, and flue gas generated by the boiler and partial fresh air are mixed through the three-way electric regulation valve and enter the liquid high-temperature aerobic fermentation reactor; odor exhausted by the liquid high-temperature aerobic fermentation reactor through the exhaust port is cooled by the odor heat exchange condenser B, then is absorbed and converted through the biological deodorization filtering tower and is discharged after reaching the standard; and hot air subjected to heat exchange of the odor heat exchange condenser B enters the hearth of the boiler to provide fresh hot air for the boiler.

In the above technical solution when the boiler is used for incinerating objects, such as garbage and dead pigs, which block the aeration heads easily, the odor and flue gas treatment system adopts the following connection modes that;

(1) in the odor treatment system of the solid high-temperature aerobic fermentation system, the exhaust port of the solid high-temperature aerobic fermentation reactor is connected with the heat exchange an inlet of the odor heat exchange condenser A; the heat exchange exhaust port of the odor heat exchange condenser A is connected with the input end of the induced draft fan; the electromagnetic valve and the bypass branch are arranged on the air inlet pipeline of the odor heat exchange condenser A; the bypass branch of the odor heat exchange condenses A is provided with the electromagnetic valve, the output end of the induced draft fan is connected with the air inlet of the biological deodorization filtering tower, and the temperature sensor is installed on the main-path an inlet pipeline of the biological deodorization filtering tower; the biological deodorization filler is arranged in the biological deodorization filtering tower; the air inlet of the odor heat exchange condenser A is connected with the atmosphere, and the air output port is connected to the air inlet of the solid high-temperature aerobic fermentation reactor through a pipeline;

(2) in the odor treatment system of the liquid high-temperature aerobic fermentation system, the exhaust flange of the liquid high-temperature aerobic fermentation reactor is connected with the heat exchange air inlet of the odor heat exchange condenser C; the heat exchange exhaust port of the odor heat exchange condenser C is connected with the input end of the induced draft fan; the electromagnetic valve and the bypass branch are arranged on the air inlet pipeline of the odor heat exchange condenser C; the bypass branch of the odor heat exchange condenser C is provided with the electromagnetic valve; the output end of the induced draft fan is connected with the air inlet of the biological deodorization filtering tower, and the temperature sensor is installed on the main-path an: inlet pipeline of the biological deodorization filtering tower; the biological deodorization filler is arranged in the biological deodorization filtering tower; the air inlet of the odor heat exchange condenser C is connected with the atmosphere; the air output port is connected to the air inlet of the aeration fan through the pipeline; and the air outlet of the aeration fan is connected with the air inlet, of the liquid high-temperature aerobic fermentation reactor; and

(3) in the flue gas treatment system of the boiler system, the flue gas exhaust port of the hot water boiler is connected with the heat exchange air inlet of the flue gas heat exchange condenser; the heat exchange exhaust port of the flue gas heat exchange condenser is connected with the input end of the induced draft fan; the output end of the induced draft fan is connected with the air inlet of the biological deodorization filtering tower; the biological deodorization filler is arranged in the biological deodorization filtering tower; the air inlet of the flue gas heat exchange condenser is connected with the atmosphere; and the air output port is connected to the air inlet of the boiler through the pipeline.

In the above technical solution, the treatment method of the odor and flue gas treatment system includes:

(1) an odor treatment method of the solid high-temperature aerobic fermentation system; the odor exhausted by the solid high-temperature aerobic fermentation reactor through the exhaust port is cooled by the odor heat exchange condenser A, then is absorbed and converted through the biological deodorization filtering tower and is discharged after reaching the standard; hot air subjected to heat exchange through the odor heat exchange condenser A enters the solid high-temperature aerobic fermentation reactor through the air inlet of the solid high-temperature aerobic fermentation reactor to provide fresh hot air for the solid high-temperature aerobic fermentation reactor:

(2) an odor treatment method of the high-temperature aerobic fermentation system, odor exhausted by the liquid high-temperature aerobic fermentation reactor through the exhaust port is cooled by the odor heat exchange condenser C, then is absorbed and converted through the biological deodorization filtering tower and is discharged after reaching the standard; hot air subjected to heat exchange of the odor heat exchange condenser C is blasted into the liquid high-temperature aerobic fermentation reactor through the aeration fan to provide fresh hot air for the liquid high-temperature aerobic fermentation reactor; and

(3) flue gas exhausted by the hot water boiler is cooled by the flue gas heat exchange condenser; then is absorbed and converted through the biological deodorization filtering tower and is discharged after reaching the standard; and cold air is heated by the flue gas heat exchange condenser and then enters the hearth of the hot water boiler to provide fresh hot air for the hot water boiler;

Further, when detecting that the odor temperature detected by the temperature sensor installed on the mam-path air inlet pipeline of the biological deodorization filtering tower is more than 40° C., the test and control system duns on the electromagnetic valves on the air inlet pipelines of the odor heat exchange condensers A, B and C and turns off the electromagnetic valves of the bypass branches to allow the odor entering the biological deodorization filtering tower to be cooled by the odor heat exchange condensers A, B and C. When detecting that the odor temperature detected by the temperature sensor installed on the main-path air inlet pipeline of the biological deodorization filtering tower is less than 15° C., the test and control system turns off the electromagnetic valves on the air inlet pipelines of the odor heat exchange condensers A, B and C and turns on the electromagnetic valves of the bypass branches to forbid the odor to enter the odor heat exchange condensers A, B and C for cooling. Therefore, the biological deodorization filtering rower works in a temperature range between 15° C. and 40° C., so as to guarantee the deodorization effect and prevent dormancy and death of microorganisms in the biological deodorization filtering tower.

In the above technical solution, when hot odor and cold air are subjected to heat exchange in the odor heat exchange condensers A, B and C, and hot flue gas and cold air are subjected to heat exchange in the flue gas heat exchange condenser, produced condensed water is discharged by the odor heat exchange condensers A, B and C and the flue gas heat exchange condenser and then is drained to the external ditch through a pipeline.

In the above technical solution, the boiler system mainly includes the boiler, the circulating pump, a pressure water tank, the three-way electric regulation valve and the electromagnetic valve. A water owlet pipeline of the boiler is connected to the input end of the three-way electric regulation valve. The two output ends of the three-way electric regulation valve are respectively connected with the water inlet flanges of the parallel-connected liquid high-temperature aerobic fermentation reactors and the parallel-connected solid high-temperature aerobic fermentation reactors through the water outlet pipeline. The water outlet pipelines of the liquid high-temperature aerobic fermentation reactors and the solid high-temperature aerobic fermentation reactors are connected with the electromagnetic valve. The water outlet of the electromagnetic valve is connected with the water return pipeline of the boiler. Temperature sensors are respectively arranged on the water outlet pipeline and the water return pipeline of the boiler. The circulating pump is further installed on the water return pipeline to allow circulating water to form a loop.

In the above technical solution, in the high-temperature aerobic fermentation reaction process, the test and control system automatically controls the opening of the circulating water three-way electric regulation valve according to the temperatures of the materials in the high-temperature aerobic fermentation reactors, so that the temperatures of the fermentation materials are stabilized at a set temperature all the time. When the temperature of the material in the high-temperature aerobic fermentation reactor of the first, fermentation object is less than the set value, the opening of the three-way electric regulation valve in this loop is 100%, and the openings in the loops of other high-temperature aerobic fermentation reactors are 0. When the temperature of the material in the first fermentation object is close to the set value, the test and control system controls to turn on the electromagnetic valve in the loop of the second high-temperature aerobic fermentation reactor, and the three-way electric regulation valve performs PID regulation to allow part of the hot circulating water to flow through the second high-temperature aerobic fermentation reactor, so that the second high-temperature aerobic fermentation reactor is heated under the condition of stabilizing the temperature of the material of the first high-temperature aerobic fermentation reactor at the set value. Because the aerobic fermentation process is a heat release process, along with the fermentation, the temperatures of the materials in the high-temperature aerobic fermentation reactors continuously rise up When the temperature of the material in the first fermentation object is greater than the set value, the test and control system slows down or shuts off the heating of the boiler. Under the action of the circulating pump, the circulating water of the first fermentation object is mixed with the circulating water of the second fermentation object, resulting in that the temperature of the material of the first fermentation object is reduced, and the temperature of the material of the second fermentation object is increased. The three-way electric regulation valve and the electromagnetic valve are coordinately controlled by the test and control system to convey fermentation reaction heat of the previous high-temperature aerobic fermentation reactor and heat generated by heating of the boiler to the second or Mth or Nth high-temperature aerobic fermentation reactor, so that the temperatures of the materials in the high-temperature aerobic fermentation reactors may be stabilized at the set value, and the heat energy generated by the fermentation reaction may be recycled.

In the above technical solution, the pressure water tank is connected with a water supplementing pipe through a valve, and the other end of the water supplementing pipe is connected with the boiler. The pressure water tank is connected with an external water supply pipe through a water supplementing valve and is configured to supplement water to a circulating water system.

Further, an exhaust valve and a presume gauge are installed on the water inlet pipeline of the circulating pump. When ah is included in the circulating water system, the air may be discharged through the exhaust valve.

In the above technical solution, the test and control system includes a sensor, a controller and a data gateway which are installed in system equipment. The controller acquires key data of all aspects of the system equipment through the sensor and coordinately controls all parts of the system according to the acquired data. The controller further communicates with the data gateway. The controller sends the key data of the system to a cloud or remote server through the data gateway for later inquiry and management.

A comprehensive treatment method based on the above system for comprehensive treatment of the cultivation pollution of the large-scale pig farm includes that:

(I) a source separation pigsty separates rainwater from sewage, separates drinking water from sewage and separates feces and urine irons pigsty flushing water; rainwater and residual drinking water of pigs are discharged to an external ditch; pigsty flushing water is conveyed into a pigsty flushing water pit; feces and urine are conveyed into a feces collection pit;

(II) when the liquid level of the pigsty flushing water in the pigsty flushing water pit reaches an overflow port the pigsty flushing water is filtered through gratings; filtrate flows into ABRs (anaerobic baffled reactors) through an overflow pipeline; the test and control system controls to turn on or turn off electromagnetic valves in front of SBRs (sequencing batch reactors) to allow liquid treated by the ABRs to respectively flow into different SBRs, and the SBRs are aerated intermittently according to an SBR technique to realize an aerobic-anaerobic technological process; before a complete SBR technique cycle is completed, the control system turns off the electromagnetic valve in horn of the reactor and turns on the electromagnetic valve in front of the next SBR; when the SBRs complete the complete SBR treatment technique, a water pump pumps supernatant into an ecological wetland for discharging; sludge in the pigsty flushing water pit, the ABRs and the SBRs are regularly conveyed to the teed port of a solid high-temperature aerobic fermentation reactor and mixed with feces for fermentation to prepare a solid organic fertilizer;

(III) the feces and urine of piglets in the suckling period and the nursing period are conveyed into a liquid high-temperature aerobic fermentation reactor, namely a solid part separated by a solid-liquid separation device from the feces and urine of fattening pigs, boars and sows is conveyed into the solid high-temperature aerobic fermentation reactor, and a liquid part separated through solid-liquid separation is conveyed into the liquid high-temperature aerobic fermentation reactor;

(IV) auxiliary materials and high-temperature aerobic bacteria are conveyed into the solid high-temperature aerobic fermentation reactor through conveying equipment; during feeding, the test and control system starts all power driving devices at the same time to allow all power supporting wheel groups to rotate at the same time to drive a horizontal drum of the solid high-temperature aerobic fermentation reactor to rotate forwards; by virtue of the action of a spiral stirring and anti-sticking device in the solid high-temperature aerobic fermentation reactor, fermentation raw materials are conveyed to the discharge side, and organic waste is shoveled up and dropped down so that the organic waste is fully stirred and mixed with oxygen, thereby enlarging the contact area of the fermentation raw materials and the oxygen;

(V) dead pigs and placentas are put into a hanging basket through a forklift truck or other sets of transferring equipment; a lifting device lifts the hanging basket into the liquid high-temperature aerobic fermentation reactor to immerse the whole hanging basket into the liquid, and at the same time, a proper amount of a composite microbial fermentation agent is inoculated into the liquid high-temperature aerobic fermentation reactor for high-temperature aerobic fermentation; if certain pig farms are qualified for treating the dead pigs and the placentas in an incineration way or other sanitary ways, the hanging basket may be omitted;

(VI) a circulating pump and a boiler are started in sequence; hot water enters a jack of the solid high-temperature aerobic fermentation reactor and a heat exchange coil of the liquid high-temperature aerobic fermentation reactor to respectively heat solids in the solid high-temperature aerobic fermentation reactor and liquid in the liquid high-temperature aerobic fermentation reactor;

(VII) a boiler system and an odor and flue gas treatment system are started at the same time, and the odor and flue gas treatment system includes the following methods:

(1) an odor treatment method of the solid high-temperature aerobic fermentation system: odor exhausted by the solid high-temperature aerobic fermentation reactor through an exhaust port is cooled by an odor heat exchange condenser A, then is absorbed and converted through a biological deodorization filtering lower and is discharged after reaching the standard; hot air subjected to heat exchange through the odor heat exchange condenser A enters the solid high-temperature aerobic fermentation reactor through an air inlet of the solid high-temperature aerobic fermentation reactor to provide fresh hot air for the solid high-temperature aerobic fermentation reactor; and

(2) an odor treatment method of a high-temperature aerobic fermentation system and a flue gas treatment method of the boiler system: an aeration fan adjusts the opening of a three-way electric regulation valve according to an oxygen demand of a material in the liquid high-temperature aerobic fermentation reactor to aerate the liquid high-temperature aerobic fermentation reactor, so that the air input ends of a hearth of the boiler and the three-way electric regulation valve are in negative pressure states all the time, and flue gas generated by the boiler and partial fresh air are mixed through the three-way electric regulation valve and enter the liquid high-temperature aerobic fermentation reactor for aeration; odor exhausted by the liquid high-temperature aerobic fermentation reactor through the exhaust port is cooled by an odor heat exchange condenser B, then is absorbed and converted through the biological deodorization filtering tower and is discharged after reaching the standard, and hot air subjected to heat exchange of the odor heat exchange condenser B enters the hearth of the boiler to provide fresh hot air for the boiler;

(VIII) when the boiler is used for incinerating objects, such as garbage and dead pigs, which block the aeration heads easily, the odor and flue gas treatment system includes the following methods:

(1) an odor treatment method of the solid high-temperature aerobic fermentation system: the odor exhausted by the solid high-temperature aerobic fermentation reactor through the exhaust port is cooled by the odor heat exchange condenser A, then is absorbed and converted through the biological deodorization filtering tower and is discharged after reaching the standard; hot an subjected to heat exchange through the odor heat exchange condenser enters the solid high-temperature aerobic fermentation reactor through the air inlet of the solid high-temperature aerobic fermentation reactor to provide fresh hot an for the solid high-temperature aerobic fermentation reactor;

(2) an odor treatment method of the liquid high-temperature aerobic fermentation system odor exhausted by the liquid high-temperature aerobic fermentation reactor through the exhaust port is cooled by the odor heat exchange condenser C, then is absorbed and converted through the biological deionization filtering tower and is discharged after reaching the standard; hot air subjected to heat exchange of the odor heat exchange condenser C is blasted into the liquid high-temperature aerobic fermentation reactor through the aeration fan to provide fresh hot air for the liquid high-temperature aerobic fermentation reactor; and

(3) flue gas exhausted by the hot water boiler is cooled by the flue gas heat exchange condenser, then is absorbed and converted through the biological deodorization filtering tower and is discharged after reaching the standard; and cold air is heated by the flue gas heat exchange condenser and then enters the hearth of the hot water boiler to provide fresh hot air for the hot water boiler.

(IX) when detecting that the odor temperature detected by a temperature sensor installed on a mam-path an inlet pipeline of the biological deodorization filtering tower is more than 40° C., the test and control system turns on the electromagnetic valves on the an inlet pipelines of the odor heat exchange condensers A, B and C and turns oil the electromagnetic valves of the bypass branches to allow the odor entering the biological deodorization filtering tower to be cooled by the odor heat exchange condensers A, B and C at first; when detecting that the odor temperature detected by the temperature sensor installed on the main-path an inlet pipeline of the biological deodorization filtering tower is less than 15° C., the test and control system turns off the electromagnetic valves on the air inlet pipelines of the odor heat exchange condensers A, B and C and turns on the electromagnetic valves of the bypass branches to forbid the odor to enter the odor heat exchange condensers A, B and C for cooling, so that the biological deodorization filtering tower works in a temperature range between 15° C. and 40° C., so as to guarantee the deodorization effect and prevent dormancy and death of microorganisms in the biological deodorization filtering tower;

(X) when hot odor and cold air are subjected to heat exchange in the odor heat exchange, condensers A, B and C, and hot flue gas and cold air are subjected to heat exchange in the flue gas heat exchange condenser, produced condensed water is discharged by the odor heat exchange condensers A, B and C and the flue gas heat exchange condenser and then is drained to an external ditch through a pipeline;

(XI) in an aerobic fermentation reaction process, the test and control system controls the power driving device of the solid high-temperature aerobic fermentation reactor to operate in a periodic intermittent operation manner of backward rotation-stop-backward rotation-stop . . . according to a detected temperature of the fermentation raw material or a set time; during rotation of the drum, under the driving of contact blocks welded on the inner wall of the horizontal drum, shoveling plates of a stirring and anti-sticking device drive materials at the inner bottom of the horizontal, drum to move upwards along the inner wall of the drum, and the materials are separated from the shoveling plates and thrown away and fall back to the bottom of the horizontal drum under the gravity action, so as to achieve stirring and air contact effects; by virtue of the action of the spiral shoveling plates in the solid high-temperature aerobic fermentation, reactor, the backward rotating drum shovels up the materials and conveys the fermentation raw materials to the feed side, so that the fermentation materials may not be compacted on a discharge side sealing cover; because cage-shaped structures of the stirring and anti-sticking device collide with different contact blocks in the drum and rotate under the driving of the contact blocks, the cage-shaped structures and the inner wall of the drum may slide relatively, so that the fermentation raw materials may not be adhered to the inner wall of the chum of the solid high-temperature aerobic fermentation reactor, and the energy consumption caused by stirring and heat conduction is minimized;

(XII) the solids in the solid high-temperature aerobic fermentation reactor are continuously fermented at 60° C. or higher for more than 24 hours to complete the whole high-temperature aerobic fermentation process to prepare the solid organic fertilizer, the test and control system controls to turn off electromagnetic valves at the front ends of the power driving device and a water inlet pipeline of the water jacket and controls to turn on a discharge gate at the same time; then the test and control system controls the power driving device to continuously rotate forwards to discharge part of old fermentation materials to the next working procedure for treatment through external conveying equipment;

(XIII) the material in the liquid high-temperature aerobic fermentation reactor is continuously fermented at 60° C. or higher for more than 3 days to complete the whole high-temperature aerobic fermentation process; if carcasses of dead pigs and placentas are not placed, the feces and urine are continuously fermented at 60° C. or above for 24 hours to complete high-temperature harmless treatment; further, the discharge port of the liquid high-temperature aerobic fermentation reactor is provided with a heat preservation anaerobic fermentation reactor (a biogas generating pith), hot fermentation liquid subjected to the high-temperature aerobic fermentation is immediately conveyed to the heat preservation anaerobic fermentation reactor subjected to heat preservation treatment through a pipeline for high-temperature or medium-temperature anaerobic fermentation; the fermentation liquid is continuously anaerobically fermented at 35-60° C. for 15 to 20 days to complete the anaerobic fermentation process; after being diluted, secondary fermentation liquid may be directly applied for agriculture, and produced biogas may be applied to the boiler system or power generation; residues produced by the dead pigs are rotten, and hairs and bone residues are conveyed to a furnace for incineration; ash produced by incineration is conveyed to the solid high-temperature aerobic fermentation reactor and is mixed with solid feces for fermentation, so as to prepare a solid organic fertilizer;

(XIV) the test and control system is used for monitoring and acquiring key data of all aspects of the comprehensive treatment system and coordinately controlling all constituents of the comprehensive treatment system according to the acquired data;

(1) in the high-temperature aerobic fermentation reaction process, the test and control system automatically controls the opening of a circulating water three-way electric regulation valve according to the temperatures of the materials in the high-temperature aerobic fermentation reactors, so that the temperatures of the fermentation materials are stabilized at a set temperature all the tune: when the temperature of the material in the high-temperature aerobic fermentation reactor of a first fermentation object is less than the set value, the opening of the three-way electric regulation valve in this loop is 100%, and the openings in the loops of other high-temperature aerobic fermentation reactors are 0; when the temperature of the material of the first fermentation object is close to the set value, the test and control system controls to turn on the electromagnetic valve in the loop of the second high-temperature aerobic fermentation reactor, and the three-way electric regulation valve performs PID regulation to allow the hot circulating water part to flow through the second high-temperature aerobic fermentation reactor, so that the second high-temperature aerobic fermentation reactor is heated under the condition of stabilizing the temperature of the material of the first high-temperature aerobic fermentation reactor at the set value: because the aerobic fermentation process is a hear release process, with the fermentation, the temperatures of the materials in the high-temperature aerobic fermentation reactors continuously rise up; when the temperature of the material in the high-temperature aerobic fermentation reactor of the first fermentation object is greater than the set value, the test and control system slows down or shuts off the heating of the boiler, under the action of the circulating pump, the circulating water of the high-temperature aerobic fermentation reactors of the first fermentation object and the second fermentation object is mixed, resulting in that the temperature of the material in the high-temperature aerobic fermentation reactor of the first fermentation object is reduced, and the temperature of the material in the high-temperature aerobic fermentation reactor of the second fermentation object is increased; the three-way electric regulation valve and the electromagnetic valve are coordinately controlled by the test and control system to convey fermentation reaction heat of the previous high-temperature aerobic fermentation reactor and treat generated by heating of the boiler to the second or Mth solid high-temperature aerobic fermentation reactor or the Nth liquid high-temperature aerobic fermentation reactor, so that the temperatures of the materials in the high-temperature aerobic fermentation reactors may be stabilized at the set value, and the heat energy generated by the fermentation reaction may be recycled; and

(2) the test and control system uploads the key data in a data region in the test and control system to a cloud or remote server for storage and backup through communication with a data gateway, so that all dam of a treatment process evidence chain are stored for later inquiry, and service staff of a remote head office can find out faults and alarm of equipment operation by virtue of the cloud data and handle with the faults and alarm in time, and the data stored in the cloud are also favorable for completion and upgrading of the treatment system.

The system and method for comprehensive treatment of the pollution of the large-scale pig farm of the present disclosure are used to comprehensively treat the feces, the urine, the pigsty flushing water, the residual drinking water of pigs, carcasses and the placentas which are produced in a breeding process of the large-scale pig farm and pollutants such as odor and flue gas, and recycle the feces, the urine, the carcasses and the placentas. In addition, by use of the heat of the odor and the flue gas, the heat efficiency is improved, and the pollution is relieved. The prevent disclosure not only treats all the pollutants in the breeding process of the large-scale pig farm, but also converts the organic wastes into available resources to conform to the circulation law of natural matters, relieves the environmental burden is relieved, provides resources are provided for repairing of the environment and has important significance for benign development of the animal husbandry and implementation of an energy sustainable development strategy.

By the adoption of the design of the source separation pigsty, the feces and urine are separated from the pigsty flushing water, the residual drinking water and the rainwater from the resource, thereby improving the feces cleaning efficiency, reducing the water use amount, maintaining nutrients of the solid feces and the liquid and improving the fertilizer efficiency of the organic fertilizer, in addition, the solid part obtained by solid-liquid separation may be directly fermented to prepare the solid organic fertilizer, thereby simplifying steps of composting pretreatment and saving a large number of dry auxiliary materials and reducing the treatment cost. Because the pigsty flushing water is not mixed with the feces and urine and is not added into the residual drinking water, the treatment volume of the pigsty flushing water is greatly reduced, and the concentrations of COD and ammonia nitrogen in the pigsty flushing water are also greatly reduced. According to the present disclosure, after the pigsty flushing water is separated from the feces and urine, impurities are removed through the gratings. Then after the pigsty flushing water is treated through the ABRs and the SBRs and then conveyed to the ecological wetland for discharging. Because the concentrations of the COD and the ammonia nitrogen in the pigsty flushing water are relatively low after the feces and urine are separated from the pigsty flushing water, the pigsty flushing water treated as above may really meet the discharge standard, and treating the pigsty flushing water in combination with the ABRs and the SBRs greatly reduces the occupation area and reduces the infrastructure construction cost, and leaves enough time for treating sewage in the SBRs, so that the treated pigsty flushing water may meet up-to-standard discharge easily, and the pigsty flushing water treatment cost is obviously reduced.

The stirring and anti-sticking device arranged in the horizontal drum of the present disclosure has the material throwing effect and may prevent the materials in the drum from being adhered to the inner wall of the drum. When the shoveling plates of the stirring and anti-sticking device and the axis of the stirring and anti-sticking device form a certain angle, a material guide effect is also achieved. The drum rotates backwards to prevent the materials from compacting the discharge side sealing cover in continuous fermentation, and the drum rotates forwards to facilitate feeding and discharging. In case of damage, the stirring and anti-sticking device may be removed for maintenance or replacement and is convenient to use and maintain.

In the present disclosure, the labyrinth sealing devices are arranged in gaps between the horizontal drum and the feed side sealing cover as well as between the horizontal drum and the discharge side sealing cover. Clearances between the feed side sealing cover and the horizontal drum as well as between the discharge side sealing cover and the horizontal drum are adjusted by adjusting the relative positions of the waist-shaped hole grooves of the feed side sealing cover and the discharge side sealing cover and the integrated base, so as to prevent material leakage from the gaps between the feed side sealing cover as well as the discharge side sealing cover and both ends of the inclined horizontal drum. The labyrinth sealing structure is simple, has no contact surface and thus avoids wear problem, and is not affected by movement and vibration of the drum.

In the present disclosure, the solid high-temperature aerobic fermentation reactor is provided with the integrated base, and the supporting wheel groups, the stop wheel, the power driving device, the feed side sealing cover and the discharge side sealing cover are fixed to the integrated base to form a standard plane, so that the relative positions of all the components may be accurately positioned, and the clearance between components may be within a reasonable range. All the power supporting wheel groups are active driving wheels, which are either supporting wheel groups or friction wheels for driving the drum to rotate, so that the transmission cost is reduced, and the horizontal drum of the solid high-temperature aerobic fermentation reactor is allowed to rotate successfully without being locked to guarantee efficient operation of the solid high-temperature aerobic fermentation reactor.

The solid high-temperature aerobic fermentation reactor is adopted to perform high-temperature aerobic fermentation on the organic wastes, so that the aerobic reaction is realized under a relatively constant high-temperature environment all the time to facilitate domestication and mass propagation of high-temperature thermophilic bacteria strains and greatly improve the fermentation efficiency. Under the constant high-temperature environment, diseased eggs are killed thoroughly, and the organic fertilizer is high in quality. Furthermore reacted odor is intensively treated and then discharged without producing secondary pollution. In a word, the method has many advantages of small occupation area, no possibility of being influenced by the environmental factors and low temperature conditions high fermentation efficiency, no secondary pollution no odor and heat emissions, good environmental protection effect and the like.

In the present disclosure, long-term and continuous high temperature is used to kill pathogenic bacteria and eggs in the pig feces and urine, the dead pigs and the placenta, so as to achieve the goal of harmless treatment. The feces and urine of the pigs and remains of the dead pigs were put into the liquid high-temperature aerobic reactor for continuous fermentation for more than 3 days at 60° C. or higher. The detection of harmful microorganisms in the liquid is shown in FIG. 26. It can be seen from the figure that the fecal conform value, the ascarid egg mortality, the bloodsucker egg mortality and the hookworm egg mortality rate all meet requirements of relevant indicators of the standard 525-2012 Organic Fertilizer and GB7959-1987 Sanitary Standard of Fecal Harmlessness. The contents of heavy metals (Cr, Cd, As, Hg and Pb) in liquid fertilizer are all within the limits of 525-2012 Organic Fertilizer. If can be seen from the table that the feces and urine of the pigs and carcasses of dead pigs are continuously fermented at 60° C. or above for more than 3 days, and most of the pathogenic microorganisms carried by the feces and mine of the pigs and the remains of the dead pigs can be killed. Therefore, the long-term and continuous high temperature is used to kill the pathogenic bacteria and eggs in the feces and urine of the pigs and the dead pigs, so as to achieve the goal of harmless treatment. The heavy metal content in liquid fertilizer does not exceed the agricultural limit standard. The above fermentation methods and test indicators show that the fermentation liquid meets the requirements of harmless treatment and meets the agricultural standards.

In the present disclosure, the feces and urine of pigs are used to proliferate thermophilic microoganisms to decompose the carcasses of the dead pigs, the placenta and the like at high speed under the high-temperature condition and rapidly transform fat, protein, sugar and the like in the carcasses of the dead pigs, the placenta and the like into soluble small-molecular organic substances dissolved in the feces and urine. This not only achieves the goal of harmless and reduced treatment after the carcasses of the dead pigs and the placenta are treated, but also increases the contents of the organic matters and nutrients in the liquid fertilizer, enhances the fertilizer efficiency and achieves the goal of resource utilization. The medium-temperature or high-temperature anaerobic fermentation is carried out by using a large amount of heat carried in the fermentation liquid subjected to high-temperature aerobic fermentation, which not only utilizes the heat carried in the fermentation liquid subjected to the high-temperature aerobic fermentation, but also greatly improves the fermentation efficiency of the anaerobic fermentation. The fermentation time is greatly shortened, and the medium-temperature or high-temperature anaerobic fermentation has higher biogas production rate than the conventional normal-temperature anaerobic fermentation, and the biogas resources can be utilized. After the anaerobic fermentation, the organic matters are further stabilized, and the stink in the feces and urine of pigs is eliminated. The quality of the liquid fertilizer and the usability of the liquid organic fertilizer are improved. Ash obtained by burning of decomposition remains is conveyed to the solid high-temperature aerobic fermentation reactor for fermentation to produce solid organic fertilizer, really realizing the goal of “zero pollution, zero emission and resource utilization”.

FIG. 27 is a report for analysis for the liquid organic fertilizer. The present disclosure fully considers the characteristics of high heat and high humidity of the organic waste fermentation odor, and creatively designs a heat exchange condenser. The hot odor of the heat exchange condenser tube is convected with the fresh air outside the tube to fully exchange heat. Compared with the traditional heat exchange mode, the method is large in heat exchange specific surface area and high in heat exchange efficiency. Meanwhile, the fresh air is heated into hot air through the heat exchange condenser, and the hot air may serve as a hot source and air oxygen source for the organic waste to heat and supply oxygen to the organic wastes, thereby shortening the organic waste fermentation and heating time and increasing the fermentation efficiency.

Due to the high humidity of the organic waste fermentation odor, a large amount of condensed water is also generated while the odor is cooled by the heat exchange condenser, and the condensed water is naturally collected into the lower end cover of the heat exchange condenser through the heat exchange pipe. When the liquid level of the condensed water teaches a certain height, the condensed water is naturally discharged into the ditch due to the pressure difference. This method is simple and feasible, and the condensed water drainage port of the lower end cover is sealed by the condensed water to prevent the condensed odor from being discharged to the atmosphere through the condensed water drainage port of the lower end cover and causing secondary pollution.

The present disclosure utilizes the odor heal exchange condenser to absorb the heat in the fermentation odor to reduce the odor temperature, and also controls the temperature range of the odor that enters the biological deodorization filtering tower through the bypass branch to avoid microbial failure and reduction of the deodorization effect due to too high or too low odor that enters the biological deionization filtering tower. The present disclosure not only ensures the deodorization effect, but also prevents the microorganisms in the biological deodorization filtering tower from sleeping and dying, and simultaneously uses the heat exchange condenser to absorb the heat in the fermentation odor or the boiler fine gas to heat the fresh air. The heated air is conveyed into the solid high-temperature aerobic fermentation reactor, the liquid high-temperature aerobic fermentation reactor or the boiler to provide fresh hot air to the materials or boilers in the high-temperature aerobic fermentation reactors to increase the efficiency and reduce energy consumption. The present disclosure utilizes the hot flue gas generated by the combustion of the boiler to aerate the feces and urine of the pigs. On one hand, a large amount of heat contained in the flue gas generated by the combustion of the boiler is absorbed by the feces and urine of pigs to heat and preserve the heat of the feces and urine of pigs, improve the fermentation efficiency and reduce the energy consumption. On the other hand, the flue gas contains a large amount of acidic gases, such as hydrogen sulfide, sulfur dioxide, sulfur trioxide, nitrogen monoxide and nitrogen dioxide, which are combined with the moisture in the feces and urine of the pigs to release H⁺ and generate neutralization reaction with OH⁻ in the feces and urine liquid of the pigs to reduce the pH of the livestock and poultry feces, reduce the release of ammonia gas, reduce the loss of nutrients in the livestock and poultry feces and also improve the fermentation efficiency.

The present disclosure provides the system and method for comprehensive treatment of the cultivation pollution of the large-scale pig farm, so as to comprehensively treat the pollution of the farm, convert the pollution into available resources, comprehensively utilize the energy generated in the conversion process and realize combination and unification of ecological benefits and economic benefits.

### DESCRIPTION OF THE DRAWINGS

FIG. 1 is a schematic diagram of a system for comprehensive treatment of cultivation pollution in a scalable pig farm;

FIG. 2 is a structural schematic diagram of a pollutant source separation pigsty;

FIG. 3 is a sectional diagram along line A-A of FIG. 2;

FIG. 4 is a schematic diagram of a pigsty flushing water treatment system;

FIG. 5 is a schematic diagram of an overall structure of a solid high-temperature aerobic fermentation reactor.

FIG. 6 is a schematic diagram of a specific structure of a solid high-temperature aerobic fermentation reactor.

FIG. 7 is a structural schematic diagram of a side surface of a power supporting wheel group;

FIG. 8 is a structural schematic diagram of a cross section of a power supporting wheel group;

FIG. 9 is a schematic diagram of a cage-shaped structure with parallel shoveling plates;

FIG. 10 is a schematic diagram of a cage-shaped structure with inclined shoveling plates;

FIG. 11 is a structural diagram of Embodiment 1 of a sealing device;

FIG. 12 is an enlarged view of portion A in FIG. 11;

FIG. 13 is a structural schematic diagram of Embodiment 2 of a sealing device;

FIG. 14 is an enlarged view of portion C in FIG. 13;

FIG. 15 is a structural schematic diagram of a stop wheel;

FIG. 16 is a structural schematic diagram of a liquid high-temperature aerobic fermentation reactor.

FIG. 17 is a structural schematic diagram of a top cover component of a liquid high-temperature aerobic fermentation reactor;

FIG. 18 is a structural schematic diagram of a hanging basket;

FIG. 19 is a schematic diagram of Embodiment 1 of an odor and flue gas treatment system;

FIG. 20 is a schematic diagram of Embodiment 2 of an odor and line gas treatment system;

FIG. 21 is a schematic diagram of Embodiment 3 of an odor and flue gas treatment system;

FIG. 22 is a schematic diagram of Embodiment 4 of an odor and flue gas treatment system;

FIG. 23 is a schematic diagram of Embodiment 5 of an odor and flue gas treatment system;

FIG. 24 is a structural schematic diagram of a heat exchange condenser;

FIG. 25 is a schematic diagram of a boiler system;

FIG. 26 is a the temperature/time table of main pathogenic microorganisms carried by feces and urine of pigs and dead pigs; and

FIG. 27 is a report for analysis for a liquid organic fertilizer.

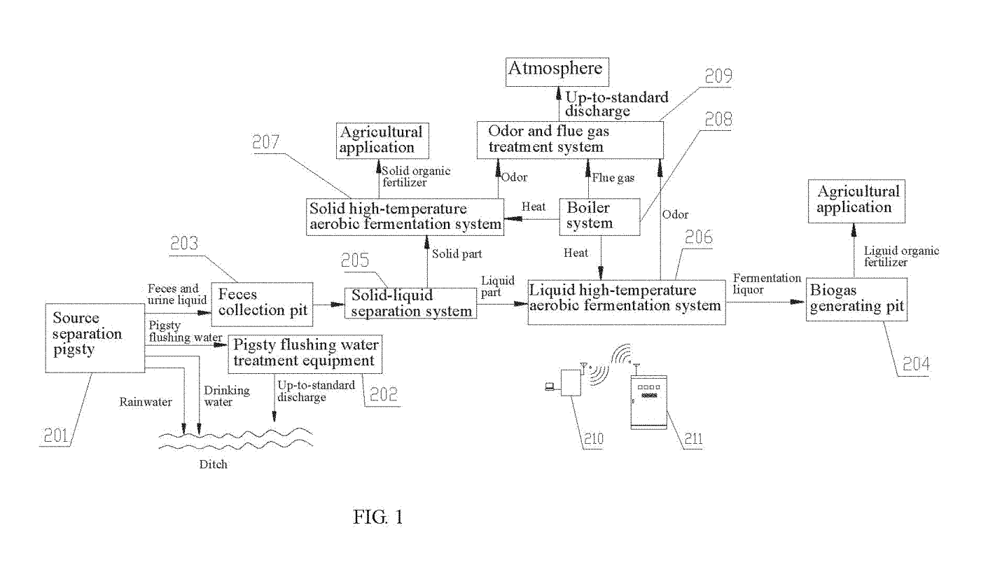

Numbering in FIG. 1: 201—source separation pigsty; 202—pigsty flushing water treatment system, 203—feces collection pit; 204—biogas generating pit; 205—solid-liquid separation system; 206—liquid high-temperature aerobic fermentation system; 207—solid high-temperature aerobic fermentation system; 208—boiler system; 209—odor and flue gas treatment system; 210—test and control system; 211—cloud or remote server.

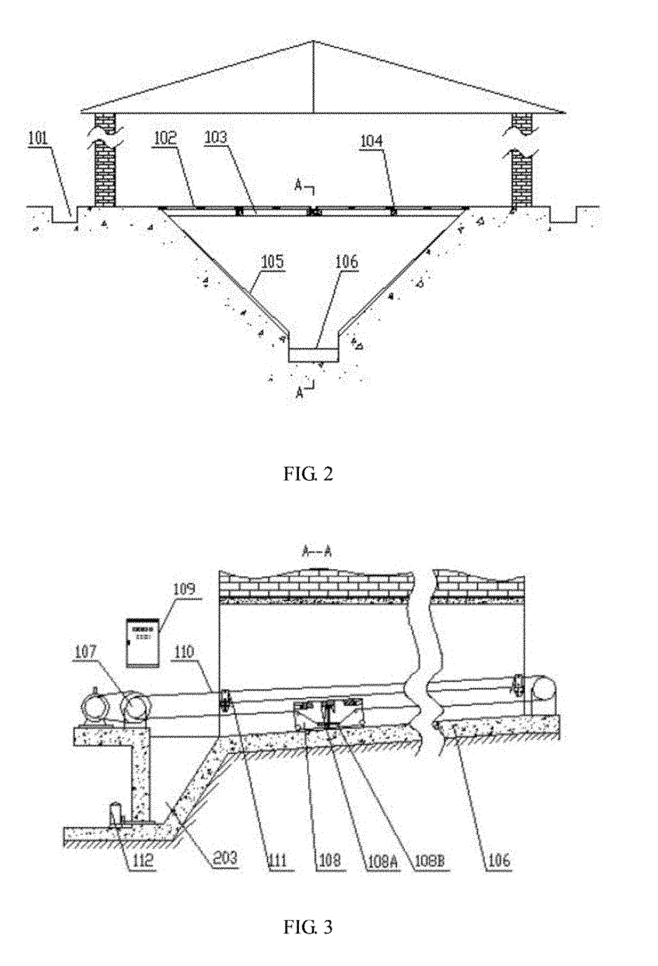

Numbering in FIG. 2 and FIG. 3: 101—external drainage ditch; 102—slatted floor; 103—longitudinal beam; 104—cross beam; 105—V-shaped slope; 106—feces cleaning ditch; 107—driving device; 108—feces scraper; 108A—limiting clip, 108B—scraper blade; 109—feces scraping control system; 110—driving rope; 111—position sensor and mounting seat; 112—sludge pump; 203—feces collection pit;

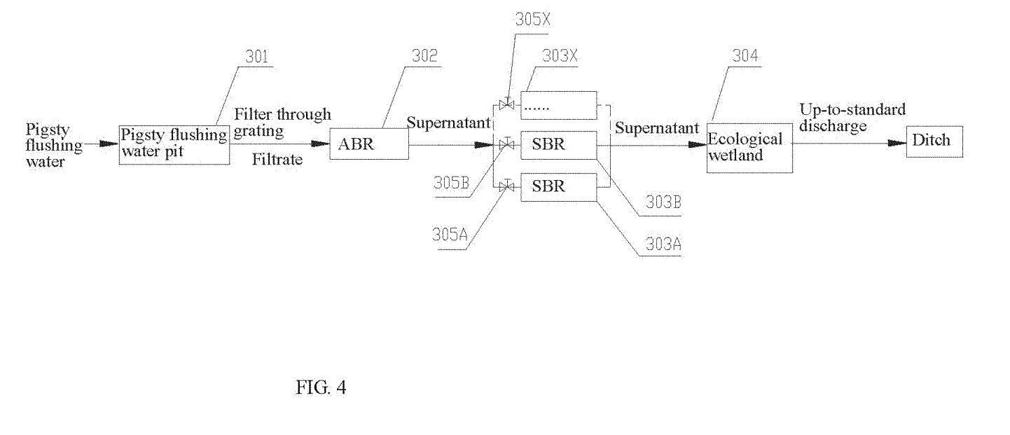

Numbering in FIG. 4: 301—pigsty flushing water pit; 302—ABR; 303A—SBR; 303B—SBR; 303X—SBR; 304—ecological wetland; 305A—electromagnetic valve; 305B—electromagnetic valve; 305X—electromagnetic valve.

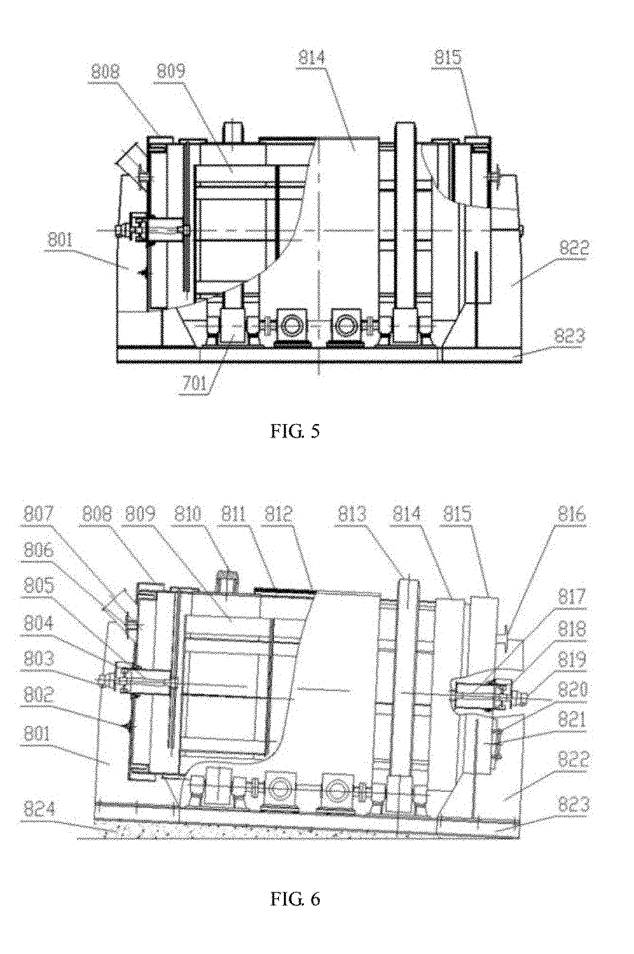

Numbering in FIG. 5: 701—power supporting wheel group; 801—feed side sealing cover; 808—feed side sealing device; 809—cage-shaped structure; 814—horizontal drum; 815—discharge side sealing device; 822—discharge side sealing cover; 823—integrated base;

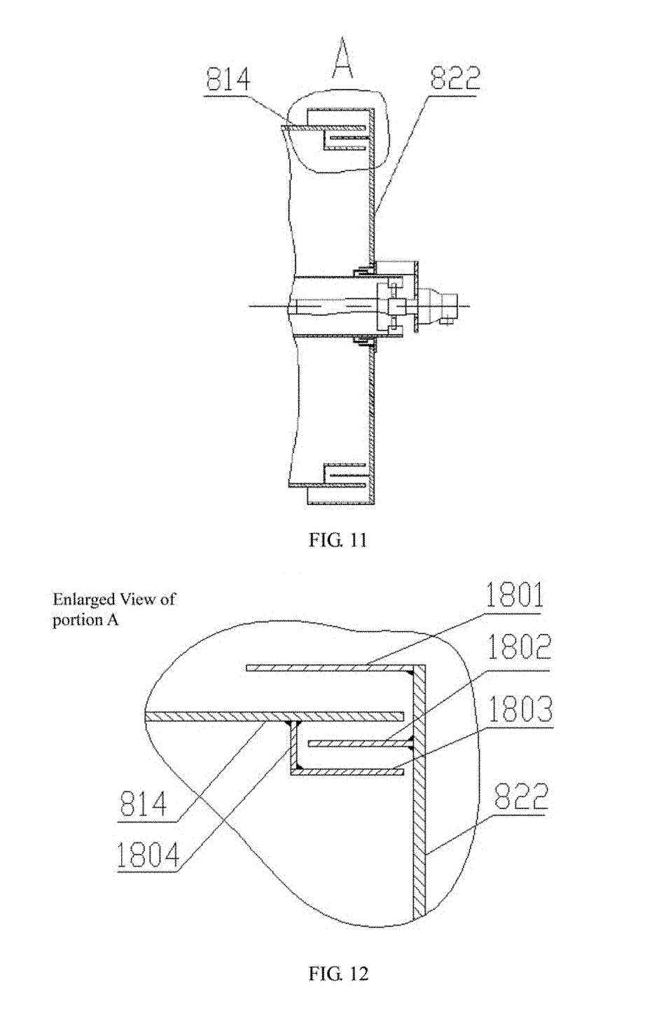

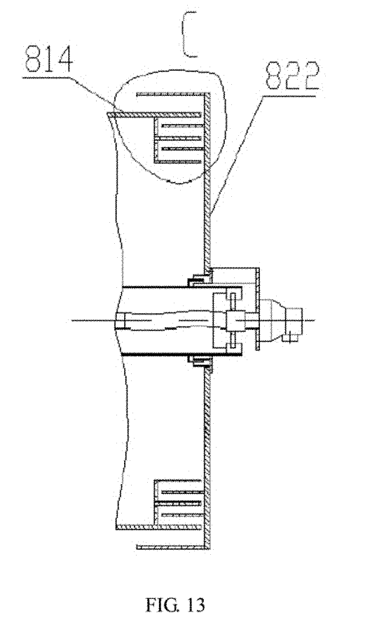

Numbering in FIG. 6, FIG. 11 and FIG. 13: 801—feed side sealing cover; 802—material temperature sensor; 803—feed side water jacket rotating joint; 804—solid high-temperature aerobic fermentation reactor water outlet flange; 805—feed side water jacket extraction pipe; 806—solid high-temperature aerobic fermentation reactor exhaust hole; 807—solid in high-temperature aerobic fermentation reactor feed hole; 808—feed side sealing device; 809—stirring and anti-sticking device; 810—feed side rolling ring; 811—water jacket; 812—heat preservation layer: 813—discharge side rolling ring; 814—horizontal drum; 815—discharge side sealing device; 816—solid high-temperature aerobic fermentation reactor air inlet hole; 817—discharge side water jacket extraction pipe; 818—solid high-temperature aerobic fermentation reactor water inlet flange; 819—discharge side water jacket rotating joint; 820—discharge gate; 821—solid high-temperature aerobic fermentation reactor discharge hole, 822—discharge side sealing cover; 823—integrated base; 824—concrete foundation;

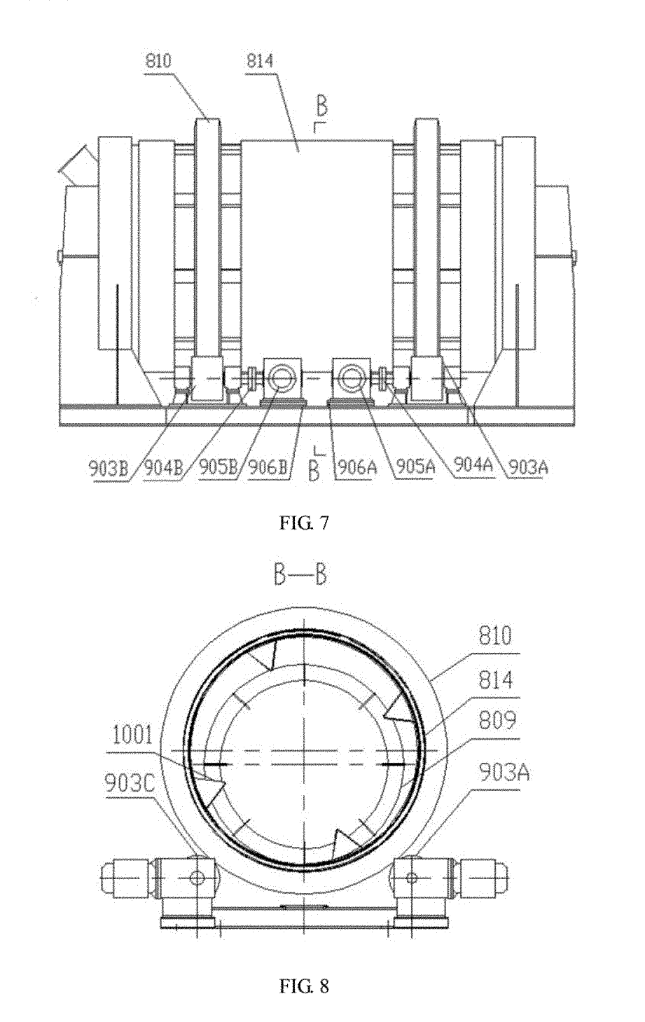

Numbering in FIG. 7: 903A—supporting wheel; 903B—supporting wheel; 904A—shaft coupler; 904B—shaft coupler; 905A—motor; 905B—motor; 906A—speed reducer; 906B—speed reducer;

Numbering in FIG. 8: 1001—contact block; 903C—supporting wheel:

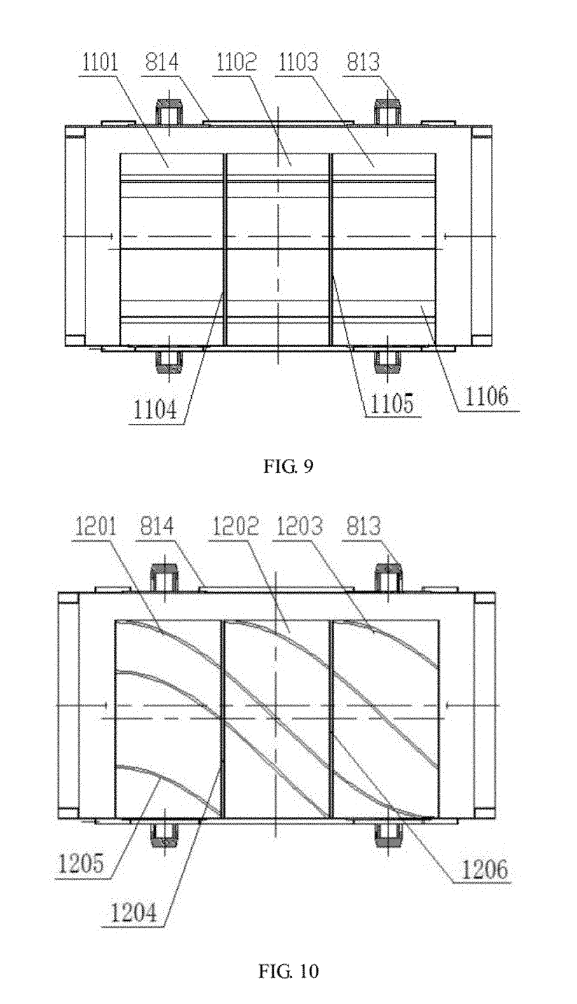

Numbering in FIG. 9: 1101—parallel shoveling plate left side cage-shaped structure; 1102—parallel shoveling plate middle side cage-shaped structure; 1103—parallel, shoveling plate right side cage-shaped structure; 1104—parallel shoveling plate middle side cage-shaped structure left supporting plate; 1105—parallel shoveling plate middle side cage-shaped structure right supporting plate; 1106—parallel shoveling plate

Numbering in FIG. 10: 1201—inclined shoveling plate left side cage-shaped structure; 1202—inclined shoveling plate middle side cage-shaped structure; 1203—inclined shoveling plate right side cage-shaped structure; 1204—inclined shoveling plate middle side cage-shaped structure left supporting plate; 1205—inclined shoveling plate; 1206—inclined shoveling plate middle side cage-shaped structure right supporting plate;

Numbering in FIG. 12: 1801—sealing cover outer hood; 1802—sealing cover inner hood A; 1803—drum ring hood A; 1804—drum lining ring;

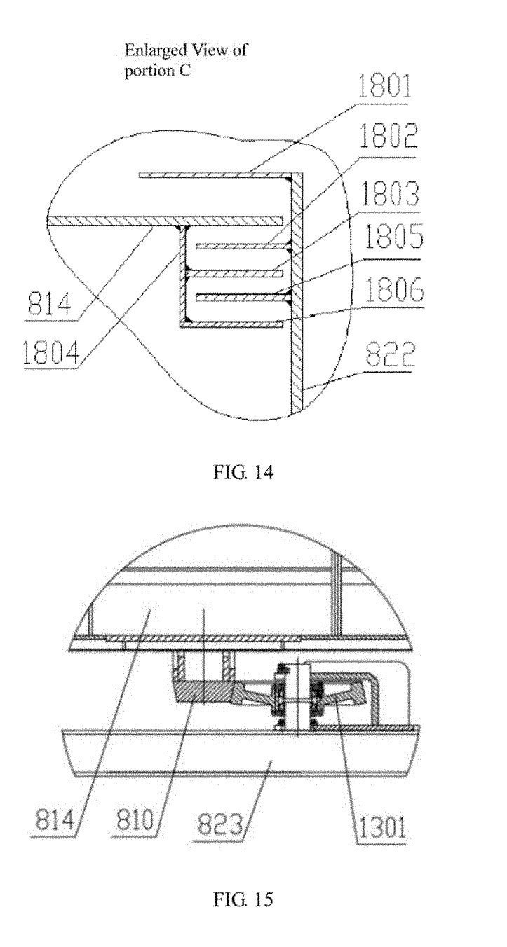

Numbering in FIG. 14: 1805—sealing cover inner hood B; 1806—drum ring hood B

Numbering in FIG. 15: 1301—stop wheel;

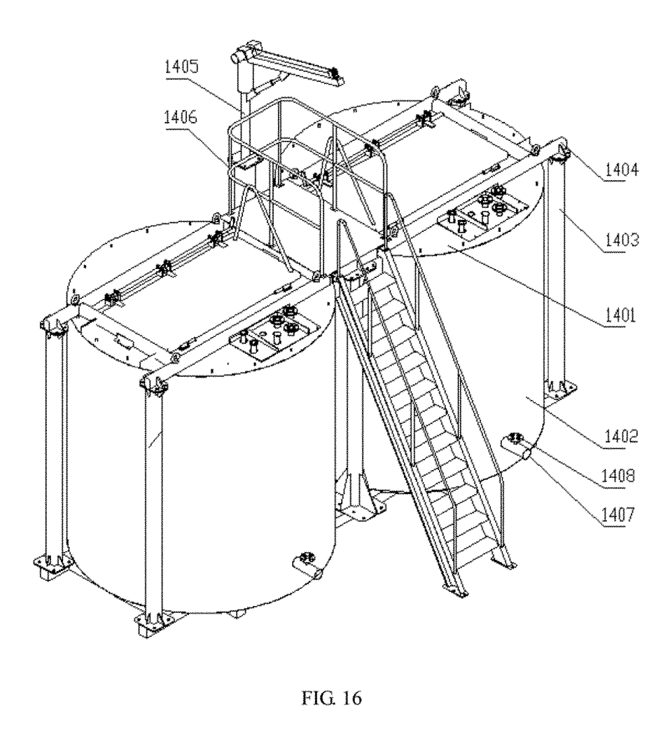

Numbering in FIG. 16: 1401—top cover; 1402—tankbody; 1403—supporting vertical column; 1404—top cover component: 1405—lifting device; 1406—escalator; 1407—liquid drainage port; 1408—liquid drainage valve;

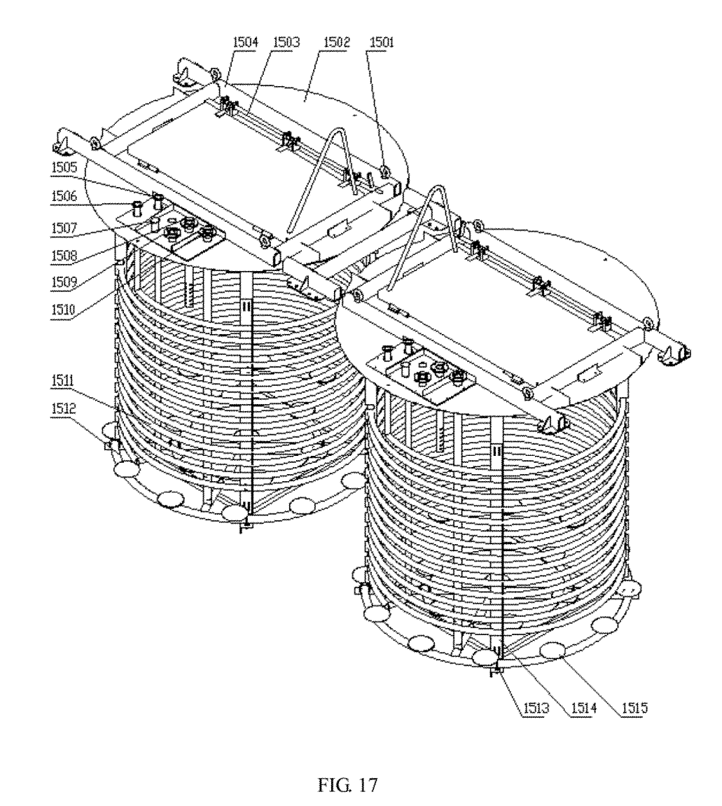

Numbering in FIG. 17: 1501—lifting ring; 1502—top cover; 1503—sealing door: 1504—bracket; 1505—heat exchange coil water outlet flange; 1506—heat exchange coil water inlet flange; 1507—safety valve; 1508—feed flange; 1509—exhaust flange; 1510—air inlet flange; 1511—heat exchange coil; 1512—aeration device; 1513—vertical frame; 1514—connecting plate; 1515—aeration head;

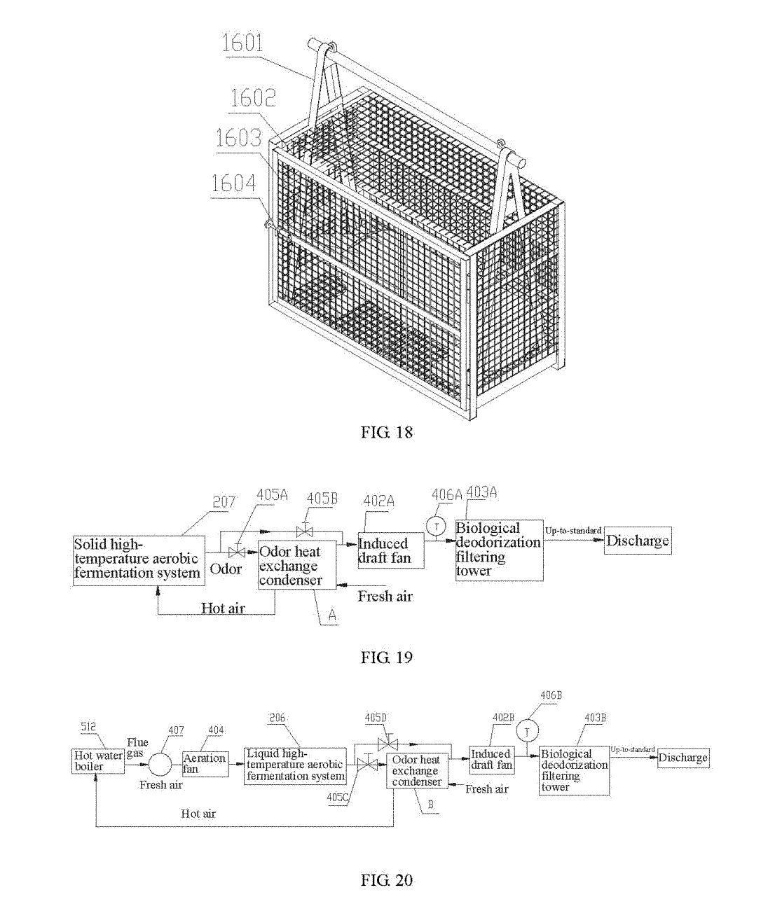

Numbering in FIG. 18: 1001—hanging basket main body; 1602—hanging basket door; 1603—lock catch; 1604—dead pig

Numbering in FIG. 19: A—odor heat exchange condenser; 402A—induced draft fan; 403A—biological deodorization filtering tower; 405A—electromagnetic valve; 405B—electromagnetic valve; 406A—temperature sensor;

Numbering in FIG. 20: B—odor heat exchange condenser; 402B—induced draft fan; 403B—biological deodorization filtering tower; 404—aeration fan; 405C—electromagnetic valve; 405D—electromagnetic valve; 406B—temperature sensor; 407—three-way electric regulation valve;

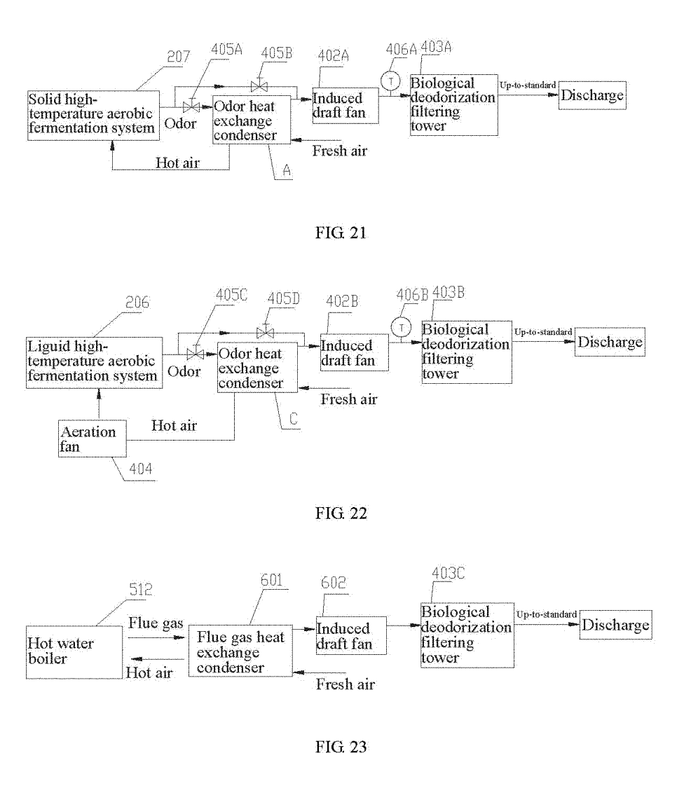

Numbering in FIG. 21: A—odor heat exchange condenser; 402—induced draft fan; 403A—biological deodorization filtering tower; 405A—electromagnetic valve; 405B—electromagnetic valve; 406A—temperature sensor;

Numbering in FIG. 22: C—odor heat exchange condenser; 602B—induced draft fan; 403B—biological deionization filtering tower; 404—aeration fan; 405C—electromagnetic valve; 405D—electromagnetic valve; 406B-temperature sensor

Numbering in FIG. 23: 601—flue gas heat exchange condenser; 602—induced draft fan; 403B—biological deodorization filtering tower; A—odor heat exchange condenser; 402A—induced draft fan; 403A—biological deodorization filtering tower 405A—electromagnetic valve; 405B—electromagnetic valve; 406A—temperature sensor

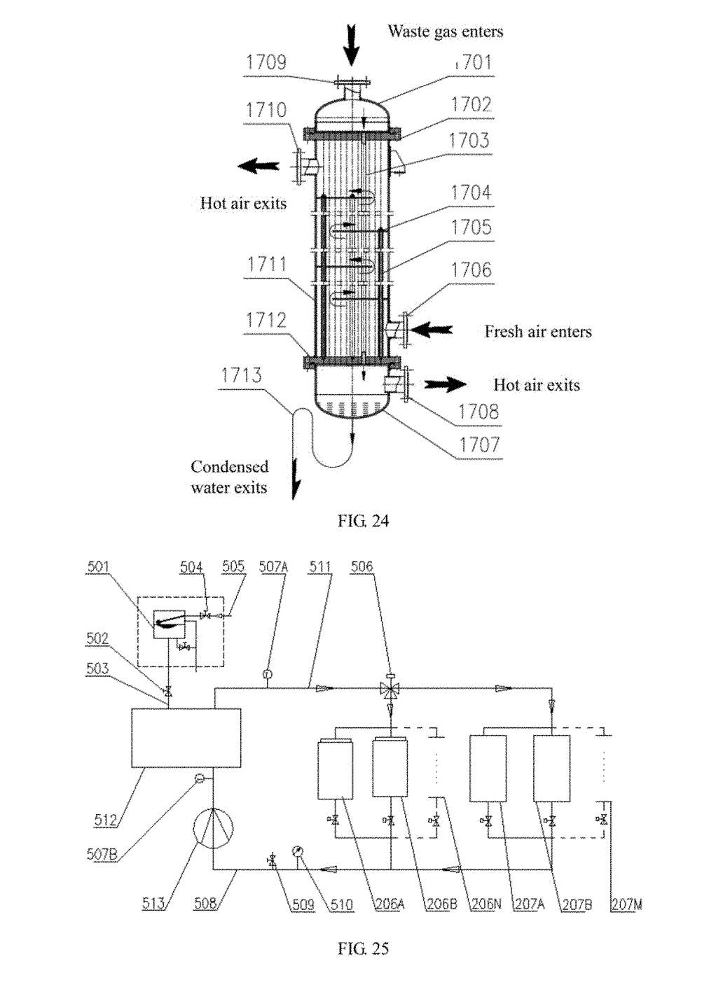

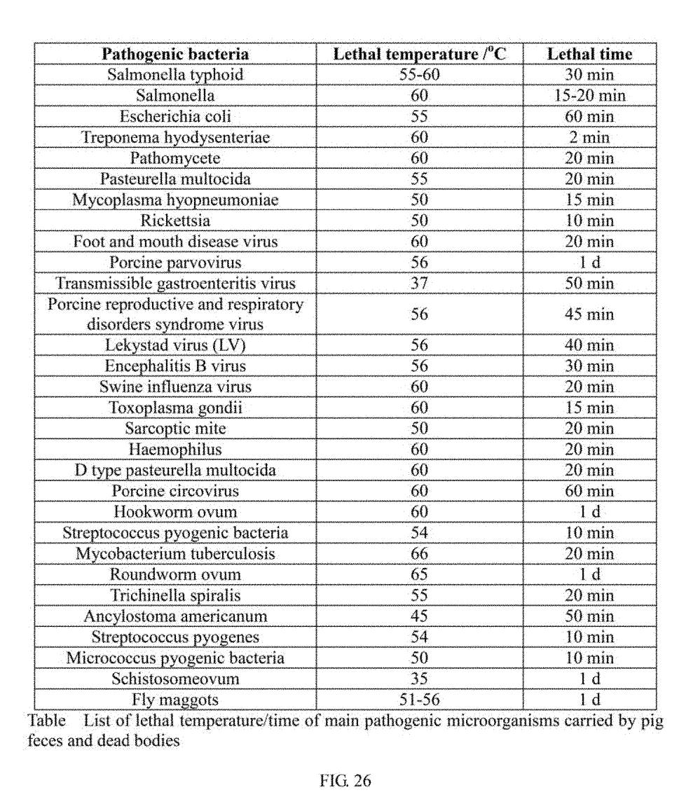

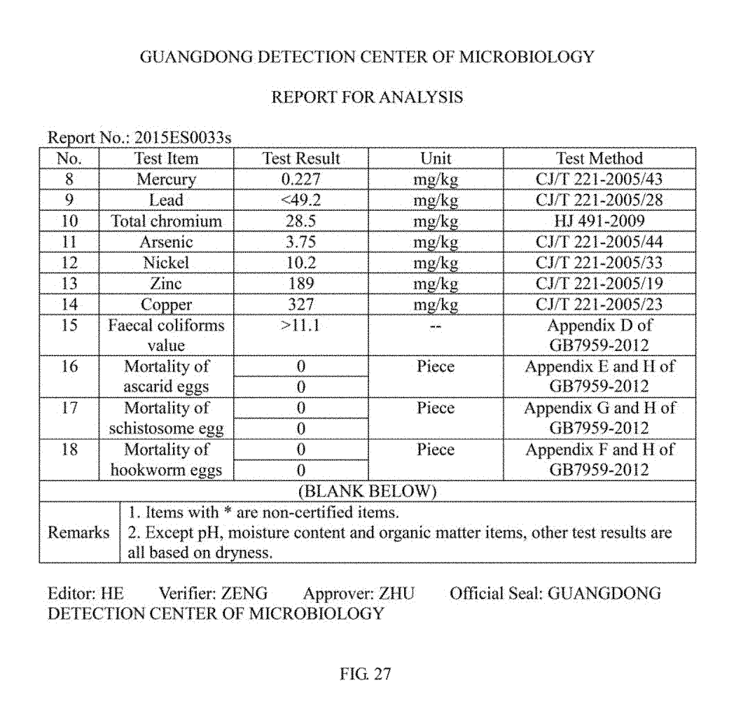

Numbering in FIG. 24: 1701—upper end cover; 1702—upper pipe plate; 1703—heat exchange pipe; 1704—partition plate; 1705—pull rod; 1706—fresh air inlet flange; 1707—lower end cover; 1708—odor exhaust flange; 1709—odor inlet flange; 1710—hot air exhaust flange; 1711—tank body; 1712—lower pipe plate; 1713—U-shaped pipe;

Numbering in FIG. 25: 206A—liquid high-temperature aerobic fermentation reactor, 206B—liquid high-temperature aerobic fermentation reactor; 206N—liquid high-temperature aerobic fermentation reactor; 207A—solid high-temperature aerobic fermentation reactor; 207B—solid high-temperature aerobic fermentation reactor; 207M—solid high-temperature aerobic fermentation reactor; 501—pressure water tank; 502—boiler water inlet valve; 503—boiler water inlet pipeline; 504—water supplementing valve; 505—water supplementing pipe; 506—three-way electric regulation valve; 507A—temperature sensor; 507B—temperature sensor; 508—boiler water return pipeline; 509—exhaust valve; 510—pressure gauge; 511—boiler water outlet pipeline; 512—boiler; 513—circulation pump

### DETAILED DESCRIPTION OF PREFERRED EMBODIMENTS

A comprehensive treatment system disclosed by the present disclosure is shown in FIG. 1, and mainly includes a source separation pigsty 201, a pigsty flushing water treatment system 202, a solid-liquid separation system 205, a liquid high-temperature aerobic fermentation system 206, a solid high-temperature aerobic fermentation system 207, a boiler system 208, an odor and flue gas treatment system 209 and a test and control system 210. The source separation pigsty 201 separates feces and urine from rainwater, pigsty flushing water and residual drinking water the rainwater and the residual drinking water are discharged into an external drainage ditch 101. The feces and urine are conveyed to a feces collection pit 203 of the solid-liquid separation system 205. The pigsty flushing water are discharged into a pigsty flushing water pit 301 of the pigsty flushing water treatment system 202. The solid-liquid separation system 205 is composed of a sludge pump installed at the bottom of the feces collection pit, a solid-liquid separation device and conveying equipment. The sludge pump pumps the feces and urine to the solid-liquid separation device. Solids separated by the solid-liquid separation device are convened to a feed port of the solid high-temperature aerobic fermentation system 207, and liquid separated by the solid-liquid separation device is conveyed to a feed port of the liquid high-temperature aerobic fermentation system 206. The liquid drainage port of tire high-temperature aerobic fermentation system 206 is connected with the liquid inlet of the biotas generating pit 204 through the conveying equipment and a pipeline. Fermentation odor generated by the solid high-temperature aerobic fermentation system 207 and the liquid high-temperature aerobic fermentation system 206 and flue gas generated by the boiler system 208 are conveyed to the odor and flue gas treatment system 209. The boiler system 208 conveys hot water to a heat exchange jacket or coil of the solid high-temperature aerobic fermentation system 207 and a jacket or coil of the liquid high-temperature aerobic fermentation system 206 through hot water pipelines so as to provide heat, and under the driving of a circulating pump 513, the hot water returns into a boiler 512 through a water return pipeline to complete the cycle. The test and control system 210 arranges sensors into all the systems to detect respective key parameters and coordinately controls the above systems.

The pollutant source separation pigsty involved in the present disclosure is shown in FIG. 2 and FIG. 3, and includes rainwater and sewage separation and drinking water and sewage separation, and separation and collection of feces and urine from pigsty flushing water. The drinking water and sewage separation is mainly composed of an autodrinker for pigs, a U-shaped water collection cavity, a drainage pipeline and an external drainage ditch. The U-shaped water collection cavity is arranged under a water faucet of the autodrinker for pigs. The drainage port in the bottom of the U-shaped water collection cavity is connected with the drainage pipeline. The water outlet of the drainage pipeline is connected with the external drainage ditch. The separation and collection of the feces and urine of pigs from the pigsty flushing water is mainly composed of a pigsty slatted floor 102, V-shaped slopes 10, a feces cleaning ditch 106, a feces scraping system and a feces collection pit 203. The V-shaped slopes 105 and the feces cleaning ditch 106 are arranged below the pigsty slatted floor 102. The V-shaped slopes 105 are positioned on both sides of the feces cleaning ditch 106. A feces scraper 108 is arranged in the feces cleaning ditch 106. A feces scraping system involved in the present disclosure is shown in FIG. 1, and mainly includes a driving device 107, a driving rope 110, the feces scraper 108, sensors 111 and a feces scraping control system 109. A limiting clip 108 A and a scraper blade 108B are arranged on the feces scraper 108. The driving device 107 is connected with the feces scraper 108 through the driving rope 110. The sensors 111 are arranged at both ends of the feces cleaning ditch 106. The driving device 107 drives the feces scraper 108 through the driving rope 110 to move back and forth from the highest end to the lowest end along the bottom surface of the feces cleaning ditch 106. When the feces scraper 108 moves towards one side of the feces collection pit 203, the limiting clip 108A clamps the scraper blade 108B, and the scraper blade 108B drives the feces and urine to move forwards to finally collect the feces and urine into the feces collection pit 203. When the sensors 111 detect that the feces scraper 108 arrives at both ends of the feces cleaning ditch 106, the feces scraping control system 109 controls the feces scraping driving device 107 to stop the operation and then to operate oppositely after delay time. When the feces scraper 108 operates oppositely the restriction of the limiting clip 108A is released, and the scraper blade 108B is shoveled up by the driving rope 110, but the feces and urine may not move backwards.

The structural schematic diagram of the pigsty flushing water treatment system involved in the present disclosure is shown in FIG. 4, and is mainly composed of a pigsty flushing water pit 301, an ABR (anaerobic baffled reactor) 302, a plurality of SBRs (sequencing batch reactors) (303A, 303B and . . . 303X). an ecological wetland 304 and a plurality of electromagnetic valves (305A, 305B and . . . 305X). Large and small gratings are arranged on the outer side of the water inlet and the inner side of the water overflow port of the pigsty flushing water pit 301. The water outlet outer side of the water overflow port of the pigsty flushing water pit is connected with the water inlet of the ABR 302 through a water outlet pipeline. The water outlet of the ABR 302 is connected to the water inlets of the parallel-connected SBRs (303A, 303B and . . . 303X) respectively through pipelines. An electromagnetic valve (305A, 305B and . . . 305X) is installed on the water inlet pipeline of each SBR (303A, 303B and . . . 303X). and the water outlets of the SBRs (303A, 303B and . . . 303X) are connected with the water inlet of the ecological wetland 304. When the source separation pigsty 201 is flushed, the pigsty flushing water enters the pigsty flushing water pit 301 through the coarse grating and flows to the ABR 302 through the fine grating and the overflow pipe. The pigsty flushing water is subjected to sludge settling and anaerobic fermentation of the ABR 302, and then fermentation liquid enters the first SBR 303A. After the liquid level of the first SBR 303A reaches its designed liquid level, the test and control system 210 controls the electromagnetic valve 305A in front of the SBR 303A to turn off the electromagnetic valve 305A in front of the first SBR 303A and tarn on the electromagnetic valve 305B of the second SBR 303B, so as to allow the liquid levels of all the SBRs to reach the designed liquid levels. The test and control system 210 realizes an aerobic-anaerobic alternating technical process on all the SBRs (303A, 303B and . . . 303X) according to an SBR technique by controlling intermittent aeration. After the SBRs (303A, 303B and . . . 303X) complete a complete SBR treatment technique, supernatant is conveyed to the ecological wetland 304 through the conveying equipment and then discharged.

The structural schematic diagram, of the solid high-temperature aerobic fermentation reactor is shown in FIG. 5 and FIG. 6. The solid high-temperature aerobic fermentation reactor is composed of an inclined horizontal drum 814, a feed side sealing cover 801, a sealing device 808, a discharge side sealing cover 822, a sealing device 815, a power supporting wheel group 701, a stirring and anti-sticking device 809 and an integrated base 823. The feed side is higher than the discharge side. The horizontal drum 814, the feed side sealing cover 801, the discharge side searing cover 822 and the sealing devices (808 and 815) on both sides form a closed fermentation space. A feed hole 807 and an exhaust hole 806 are formed in the upper part of the feed side sealing cover 801. An air inlet hole 816 is formed in the upper part of the discharge side sealing cover 822. A discharge hose 821 is formed in the lower part of the discharge side sealing cover 822. A discharge gate 820 is installed on the discharge hole. A temperature sensor 802 is arranged at the lower part of the feed side sealing cover 801. A temperature probe of the temperature sensor 802 extends into the horizontal drum 814.

A water jacket 811 is welded outside the horizontal drum 814, and is divided into several parts by a feed side rolling ring 810 and a discharge side rolling ring 813 on the horizontal drum 814. The water jacket 811 is connected into a whole through a water jacket connection pipe. The water jacket 811 is connected with a solid high-temperature aerobic fermentation reactor water inlet flange 818 by a feed side water jacket extraction pipe 805 through a feed side water jacket rotating joint 803 arranged in the center of the feed side sealing cover 801. The water jacket 811 is connected with a solid high-temperature aerobic fermentation reactor water outlet flange 804 by a discharge side water jacket extraction pipe 817 through a discharge side water jacket rotating joint 819 arranged in the center of the discharge side sealing cover 822. The water inlet flange 818 and the water outlet flange 804 of the solid high-temperature aerobic fermentation reactor are connected with a water outlet pipeline 511 of the boiler system 208 to form a circulating loop. A heat preservation layer 812 is arranged outside the water jacket 811, so that radiation waste of heat energy may be reduced.

The stirring and anti-sticking device 809 is positioned in the horizontal drum 814. The horizontal drum 814 is arranged on the power supporting wheel group 701. The power supporting wheel group 701, the feed side sealing cover 801 and the discharge side sealing cover 822 are fixed to the inclined integrated base 823 to form a whole. The integrated base 823 is fixed to an inclined concrete foundation 824 through second pouring; the basic plane of the concrete foundation 824 and the gradient of a horizontal plane form an adjustable included angle of 0-5 degrees. The conveying speed of fermentation raw materials to the discharge end may be adjusted by adjusting the included angle.

The structural schematic diagram of the side surface and the structural schematic diagram of the cross section of the power supporting wheel group 701 are shown in FIG. 7 and FIG. 8. The power supporting wheel group 701 is composed of two groups of supporting wheels and power driving devices thereof and the like. Power driving adopts four-wheel driving. In FIG. 6 of the structural schematic diagram of the side surface, the structure of the first power driving device is that; a motor 905A, a speed reducer 906A and a shaft coupler 904A are connected with the supporting wheel 903A in sequence and are in connection transmission in sequence. The structure of the second power driving device is that: a motor 905B, a speed reducer 906B and a shaft coupler 904B are connected with the supporting wheel 903B in sequence and are in connection transmission in sequence. In this way, each supporting wheel is a driving wheel. The two groups of supporting wheels are in linear contact with the rolling ring 901 of the horizontal drum 814. The power supporting wheel group is controlled to coordinately drive the horizontal, drum 814 to rotate.

The structural schematic diagram of a contact block is shown in FIG. 8. A general shovel plate structure is not disposed on the inner wall of the horizontal drum 814. On the inner wall, a plurality of contact blocks 1001 are uniformly fixed relative to gap positions of cage-shaped structures 809 of the stirring and anti-sticking device. When the horizontal drum 814 rotates, the contact blocks 1001 on the inner wall drive a parallel shoveling plate left side cage-shaped structure 1101, a parallel shoveling plate middle side cage-shaped structure 1102 and a parallel shoveling plate right side cage-shaped structure 1103 to rotate at the same time. Because shoveling plates 1106 of the cage-shaped structures have certain widths, the three cage-shaped structures (1101, 1102 and 1103) drive materials at the bottom of the horizontal drum 814 to move upwards. Under the gravity action, the materials are separated from the shoveling plates and thrown away, and fall to the bottom of the horizontal drum 814, so as to achieve material throwing and stirring effects. Because the outer diameters of the parallel shoveling plate left, side cage-shaped structure 1101, the parallel shoveling plate middle side cage-shaped structure 1102 and the parallel shoveling plate right side cage-shaped structure 1103 are less than the inner diameter of the horizontal drum 814, gaps are also reserved between the contact blocks 1001 and the three cage-shaped structures (1101, 1102 and 1103). When the horizontal drum 814 rotates, the three cage-shaped structures (1101, 1102 and 1103) and the horizontal drum 814 generate relative movement. By virtue of collision and scratching between left supporting plates and right supporting plates of the three cage-shaped structures (1101, 1102 and 1103) as well as between the shoveling plates and the inner wall of the horizontal drum 814, the materials possibly adhered on the inner surface of a drum body of the horizontal drum 314 may be cleaned, so as to achieve an effect of preventing the materials in the horizontal drum 814 from being adhered on the inner wall of the horizontal drum 814.

The stirring and anti-sticking system 809 is composed of one or more than one cage-shaped structure. According to a state whether the axes of the cage-shaped structures are parallel to the shoveling plates, the cage-shaped structures are divided into parallel shoveling plate cage-shaped structures and inclined shoveling plate cage-shaped structures. The schematic diagram of the parallel shoveling plate cage-shaped structure is shown in FIG. 9. The stirring and anti-sticking system is composed of the parallel shoveling plate left side cage-shaped structure 1101, the parallel shoveling plate middle side cage-shaped structure 1102 and the parallel shoveling plate right side cage-shaped structure 1103. Each cage-shaped structure is composed of a left supporting plate, a right supporting plate and a plurality of shoveling plates. The left and the right supporting plates are circular rings and are coaxial. The plurality of shoveling plates are arranged between the supporting plates. As shown in FIG. 11, the parallel shoveling plate middle side cage-shaped structure 1102 is composed of a parallel shoveling plate middle side cage-shaped structure left supporting plate 1104, a parallel shoveling plate middle side cage-shaped structure right supporting plate 1105 and a plurality of shoveling plates 1106. The left supporting plate 1104 and the right supporting plate 1105 are coaxial, and a plurality of parallel shoveling plates 1106 are arranged between the left supporting plate 1104 and the right supporting plate 1105 The shoveling plates 1106 are parallel to the axis of the horizontal drum 814.

The schematic diagram of the inclined shoveling plate cage-shaped structure is shown in FIG. 10. The stirring and anti-sticking system 809 is composed of an inclined shoveling plate left side cage-shaped structure 1201, an inclined shoveling plate middle side cage-shaped structure 1202 and an inclined shoveling plate right side cage-shaped structure 1203. Each cage-shaped structure is composed of a left supporting plate, a right supporting plate and a plurality of inclined shoveling plates. The left and right supporting plates are circular rings and are coaxial. The plurality of inclined shoveling plates are disposed between the supporting plates. The inclined shoveling plates are inclined to their axes at certain angles. The inclined shoveling plate middle side cage-shaped structure 1202 is composed of a left supporting plate 1204, a right supporting plate 1205 and a plurality of inclined shoveling plates 1206. When the horizontal chum 902 rotates, the contact blocks 1001 on the inner wall drive the inclined shoveling plate left side cage-shaped structure 1201, the inclined shoveling plate middle side cage-shaped structure 1202 and the inclined shoveling plate right side cage-shaped structure 1203 to rotate at the same time. Because the shoveling plates 1206 of the cage-shaped structures have certain widths, the three cage-shaped structures (1201, 1202 and 1203) drive materials at the bottom of the horizontal drum 814 to move upwards. Under the gravity action, the materials are separated from the shoveling plates and thrown away, and fall to the bottom of the horizontal drum 814. When the materials are thrown away, the shoveling plates of the three cage-shaped structures (1201, 1202 and 1203) form certain angles to their axes, and a forward thrust is generated while the materials are thrown away to allow the materials to move from the feed side to the discharge side, so as to achieve material throwing, stirring and guiding effects.

The structural schematic diagram of the labyrinth sealing device involved in the present disclosure is shown in FIG. 11 and FIG. 12. The structure of the sealing device is sealed in a labyrinth manner. Sealing between the drum 814 and the teed side sealing cover 801 and sealing between the drum 814 and the discharge side sealing cover 822 are labyrinth sealing. The labyrinth sealing is realized on the inner sides of the two sealing covers (the feed side sealing cover 801 and the discharge side sealing cover 822). As shown in FIG. 10, an outer hood 1801 and an inner hood 1802 which are coaxial with each other are perpendicularly welded on the inner side of the discharge side sealing cover 822. Correspondingly, coaxial lining rings 1804 are welded in parts, positioned on both sides, of the drum 814. The perpendicular lining rings 1804 are welded with coaxial ring hoods 1803, each of which has an outer diameter less than the inner diameter of the drum 814. The inner diameter of the sealing cover outer hood 1801 is greater than the outer diameter of the drum 814. The inner diameter of the sealing cover inner hood 1802 is greater than the outer diameter of the ring hood 1803. The outer diameter of the sealing cover inner hood 1802 is less than the inner diameter of the drum 814. The depth of the sealing cover inner hood 1802 is equal to that of the ring hood 1803. The labyrinth sealing effect is guaranteed by gaps between the inner sides of the sealing covers (the feed side sealing cover 801 and the discharge side sealing cover 822) and the end surfaces of the drum 814. If the gaps between the inner sides of the sealing covers (the feed side sealing cover 801 and the discharge side sealing cover 822) and the end surfaces of the drum 814 are smaller, fewer materials are leaked, so that the positions of the end covers (the feed side sealing cover 801 and the discharge side sealing cover 822) on both sides are adjusted to allow the drum 814 to rotate flexibly, so as to achieve a sealing effect of least leakage.

Further, the number of labyrinths is increased to lengthen the labyrinths and reduce the leakage. As shown in FIG. 13 and FIG. 14, an outer hood 1801, an inner hood A1802 and an inner hood B1805 which are coaxial are perpendicularly welded on the inner side of the discharge side sealing cover 822. Correspondingly, coaxial lining rings 1804 are welded in pans, positioned on both sides, of the drum 814. The perpendicular lining rings 1804 are welded with a ring hood A1803 and a ring hood B1806 coaxial with each other and having an outer diameter less than the inner diameter of the drum 814. The inner diameter of the sealing cover outer hood 1801 is greater than the outer diameter of the drum 814. The inner diameter of the sealing cover inner hood A1802 is greater than the outer diameter of the ring hood A1803. The outer diameter of the sealing cover inner hood A1802 is less than the inner diameter of the drum 814. The inner diameter of the sealing cover inner hood B1805 is greater than the outer diameter of the ring hood B1803. The inner diameter of the ring hood A1802 is greater than the outer diameter of the sealing cover inner hood B1805. The depth of the sealing cover inner hood A1802 is equal to those of the sealing cover inner hood B1805, the ring cover A1803 and the ring cover B1806, that is, the four hoods have the consistent depths. The labyrinth sealing effect is guaranteed by gaps between the inner sides of the sealing covers (the feed side sealing cover 801 and the discharge side sealing cover 822) and the end surfaces of the drum 814. If the gaps between the inner sides of the sealing covers (the feed side sealing cover 801 and the discharge side sealing cover 822) and the end surfaces of the drum 814 are smaller, fewer materials are leaked, so that the positions of the end covers (the feed side sealing cover 801 and the discharge side sealing cover 822) on both sides ate adjusted to allow the drum 814 to rotate flexibly, so as to achieve a sealing effect of least leakage.

The structural schematic diagram of a stop wheel is shown in FIG. 15. The stop wheel 1301 is connected to the integrated base 823 in a bolting manner. A waist-shaped hole groove is formed in a stop wheel seat. The stop wheel 1301 is adjusted through the waist-shaped hole groove so that the stop wheel 1301 comes into contact with the side line of the discharge side rolling ring 813. The stop wheel 1301 keeps off an axial component force of the horizontal drum 814, so as to prevent the horizontal drum 814 from moving along the axis.

The liquid high-temperature aerobic fermentation reactor is of a vertical type structure. As shown in FIG. 16, each liquid high-temperature aerobic fermentation reactor 1407 mainly includes a tank body 1402, a supporting vertical column 1403, a top cover component 1404, a lifting device 1405, a hanging basket and the like. The structural diagram of the top cove component 1404 of the liquid high-temperature aerobic fermentation reactor is shown in FIG. 14. The fop cover component 1404 mainly includes a lifting ring 1501, a top cover 1502, a sealing door 1503, a heat exchange coil water outlet flange 1505, a heat exchange coil water inlet flange 1506, a safety valve 1507, a feed flange 1505, an exhaust flange 1509. an air inlet flange 1510, a heat exchange coil 1511 and an aeration device 1512. The lifting ring 1501 is fixed to the top cover 1502 and is used for lifting the top cover component 1404. The feed flange 1508, the water inlet flange 1506, the water outlet flange 1505, the aeration device 1512, the air inlet flange 1510 and the exhaust flange 1509 are fixed to the top cover 1502. The heat exchange coil 1511 is fixed to the lower side of the top cover 1502 through a connecting plate 1514 and a vertical frame 1513 and is immersed in the fermentation liquid. The top cover 1502 is supported by the supporting vertical column 1403 and fixed at the upper part of the liquid high-temperature aerobic fermentation reactor 1407 to form a closed space together with the tank body 1402. A liquid drainage port 1407 is formed in the bottom of the tank body 1402. The liquid drainage port 1407 is connected to the biomass generating pit 212 through a pipeline. A liquid drainage valve 1408 is arranged on a liquid drainage pipeline. The aeration device 1512 is connected to an external aeration fan through an air inlet pipeline. A plurality of aeration heads 1515 are uniformly arranged on an aeration pipeline.

The structure of the hanging basket is shown in FIG. 18. The hanging basket is a tool used for accommodating dead pigs 1604 and placentas, and is mainly composed of a hanging basket main body 1601, a hanging basket door 1602, a lock catch 1603 and the like.

The schematic diagram of the odor and flue gas system involved in the present disclosure is shown in FIG. 19 FIG. 20, FIG. 21, FIG. 22 and FIG. 23. The odor and flue gas treatment system mainly includes odor heat exchange condensers (A, B and C), induced draft fans (402A and 402B), biological deodorization filtering towers (403A and 403B), an aeration fan 404, an electromagnetic valve 405, a three-way electromagnetic valve 407, a flue gas heat exchange condenser 601 and an induced draft fan 602. The schematic diagram of the odor treatment system of the solid high-temperature aerobic fermentation system is shown in FIG. 19. The exhaust port of the solid high-temperature aerobic fermentation reactor 207 is connected with the heat exchange an inlet of the odor heat exchange condenser A. The heat exchange exhaust port of the odor heat exchange condenser A is connected with the input end of the induced draft fan 402A. The electromagnetic valve 405A and a bypass branch are arranged on the air inlet pipeline of the odor heat exchange condenses A. The bypass branch of the odor heat exchange condenser A is provided with the electromagnetic valve 405B. The output end of the induced draft foil 402A is connected with an air inlet of the biological deodorization filtering tower 403A, and a temperature sensor 406A is installed on a main-path air inlet pipeline of the biological deionization filtering towel 403A. A biological deodorization filler is arranged in the biological deodorization filtering tower 403A. Odor exhausted by the solid high-temperature aerobic fermentation reactor 307 is cooled by the odor heat exchange condenser A, then is absorbed and converted through the biological deodorization filtering tower 403A and is discharged after reaching the standard. The air inlet of the odor heat exchange condenser A is connected with the atmosphere, and the air output port is connected to the air inlet of the solid high-temperature aerobic fermentation reactor 207 through a pipeline. Cold air is heated by the odor heat exchange condenser A to provide fresh hot air for the solid high-temperature aerobic fermentation reactor 207.

The odor treatment system of the liquid high-temperature aerobic fermentation system and the flue gas treatment system of the boiler system are as shown in FIG. 20. The flue gas exhaust port of the hot water boiler 512 is connected with one air inlet input end of the three-way electric regulation valve 407, and the other air inlet input end of the three-way electric regulation valve 407 is connected with the atmosphere. The output end of the three-way electric legislation valve 407 is connected with the input end of an aeration fan 404. The output end of the aeration fan 404 is connected with the air inlet flange of the liquid high-temperature aerobic fermentation reactor 206. The exhaust flange of the liquid high-temperature aerobic fermentation reactor 206 is connected with a heat exchange an inlet of the odor heat exchange condenser B. A heat exchange exhaust port of the odor heat exchange condenser B is connected with the input end of the induced draft fan 402B. An electromagnetic valve 405C and a bypass branch are arranged on an air inlet pipeline of the odor heat exchange condenser B. The bypass branch of the odor heat exchange condenser B is provided with an electromagnetic valve 405D. The output end of the induced draft fan 402B is connected with an air inlet of the biological deodorization filtering tower 403B, and a temperature sensor 406B is installed on a main-path air inlet pipeline of the biological deodorization filtering tower 403B. A biological deodorization filler is arranged in the biological deodorization filtering tower 403B. The aeration fan 404 adjusts the opening of the three-way electric regulation valve 407 according to an oxygen demand of a material in the liquid high-temperature aerobic fermentation reactor 206 to aerate the liquid high-temperature aerobic fermentation reactor 206, so that the an input ends of a hearth of the hot water boiler 512 and the three-way electric regulation valve 407 are in negative pressure states all the time, and flue gas generated by the hot water boiler 512 and partial fresh air are mixed through the three-way electric regulation valve 407 and enter the liquid high-temperature aerobic fermentation reactor 206. Odor exhausted by the liquid high-temperature aerobic fermentation reactor 206 is cooled by the odor heat exchange condenser B, then is absorbed and converted through the biological deodorization filtering tower 403B and is discharged after reaching the standard. The air inlet of the odor heat exchange condenser B is connected with the atmosphere, and the air output port is connected to the air inlet of an air blower of the hot water boiler 512. Cold air is heated by the odor heat exchange condenser B to provide fresh hot air for the hot water boiler 512.

When the boiler is used for incinerating objects, such as garbage and dead pigs, which block the aeration heads easily, the odor and flue gas treatment system is shown in FIG. 21, FIG. 22 and FIG. 23. The schematic diagram of the odor treatment system of the solid high-temperature aerobic fermentation system is shown in FIG. 17-1. The exhaust port of the solid high-temperature aerobic fermentation reactor 207 is connected with the heat exchange air inlet of the odor heat exchange condenser A. The heat exchange exhaust port of the odor heat exchange condenser A is connected with the input end of the induced draft fan 402A. The electromagnetic valve 405A and a bypass branch are arranged on the air inlet pipeline of the odor heat exchange condenser A. The bypass branch of the odor heat exchange condenser A is provided with the electromagnetic valve 405B. The output end of the induced draft fan 402A is connected with an air inlet of the biological deodorization filtering tower 403A, and a temperature sensor 406A is installed on a main-path air inlet pipeline of the biological deodorization filtering tower 403A. A biological deodorization filler is arranged in the biological deodorization filtering rower 403A. Odor exhausted by the solid high-temperature aerobic fermentation reactor 207 is cooled by the odor heat exchange condenser A, then is absorbed and converted through the biological, deodorization filtering tower 403A and is discharged after reaching the standard. The air inlet of the odor heat exchange condenser A is connected with the atmosphere, and the air output port is connected to the air inlet of the solid high-temperature aerobic, fermentation reactor 207 through a pipeline. Cold air is heated by the odor heat exchange condenser A to provide fresh hot air for the solid high-temperature aerobic fermentation reactor 207.

The schematic diagram of the odor treatment system of the liquid high-temperature aerobic fermentation system is shown in FIG. 22. The exhaust flange of the liquid high-temperature aerobic fermentation reactor 206 is connected with a heat exchange air inlet of the odor heat exchange condenser C. A heat exchange exhaust port of the odor heat exchange condenser C is connected with the input end of the induced draft fair 402B. An electromagnetic valve 405C and a bypass branch are arranged on an air inlet pipeline of the odor heat exchange condenser C. The bypass branch of the odor heat exchange condenser C is provided with an electromagnetic valve 405D. The output end of the induced draft fan 402B is connected with an air inlet of the biological deodorization filtering tower 403B, and a temperature sensor 406B is installed on a main-path air inlet pipeline of the biological deodorization filtering tower 403B. A biological deodorization filler is arranged in the biological deodorization filtering tower 403B. Odor exhausted by the liquid high-temperature aerobic fermentation reactor 206 is cooled by the odor heat exchange condenser C, then is absorbed and converted through the biological deodorization filtering tower 403B and is discharged after reaching the standard. The air inlet of the odor heat exchange condenser C is connected with the atmosphere, and the air output port is connected to the air inlet of the aeration fan 404. The air outlet of the aeration fan 404 is connected with the air inlet of the liquid high-temperature aerobic fermentation reactor 206. Cold air is heated by the odor heat exchange condenser C and then blown into the liquid high-temperature aerobic fermentation reactor 206 through the aeration fan 404 to provide fresh hot air for the liquid high-temperature aerobic fermentation reactor 206.

The schematic diagram of the flue gas treatment system of the boiler system is shown in FIG. 23. The flue gas exhaust port of the boiler 512 is connected with the heat exchange air inlet of the flue gas heat exchange condenser 601. The heat exchange exhaust port of the flue gas heat exchange condenser 601 is connected with the input end of the induced draft fan 602. The output end of the induced draft fan 602 is connected with the air inlet of the biological deodorization filtering tower 403C. A biological deodorization filler is arranged in the biological deodorization filtering tower 403C. Flue gas exhausted by the hot water boiler 512 is cooled by the Hue gas heat exchange condenser 601, then is absorbed and converted through the biological deodorization filtering tower 403C and is discharged after reaching the standard. The air inlet of the flue gas heat exchange condenser 601 is connected with the atmosphere, and the air output port is connected to the air inlet of the air blower of the hot water boiler 512 through the pipeline. Cold air is heated by the flue gas heat exchange condenser 601 and then blown into the hearth of the hot water boiler 512 through the air blower of the hot water boiler 512 to provide fresh hot air for the hot water boiler 512.

The structure schematic diagram of the heat exchange condenser involved in the present disclosure is shown in FIG. 24, the heat exchange condenser includes an upper end cover 1701, a tank body 1711 and a lower end cover 1707 winch are connected and fixed in sequence. The upper end of the upper end cover 1701 has an odor inlet flange 1709. An odor collection pipeline is connected and fixed with the odor inlet flange 1709. The lower part of the side wall of the tank body 1711 is provided with a fresh air inlet flange 1706, and the upper part is provided with a hot air exhaust flange 1710. An upper pipe plate 1702 is installed at the upper part of the tank body 1711, and a lower pipe plate 1712 is installed at the lower part of the tank body 1711. A plurality of holes are uniformly formed in the upper pipe plate 1702 and the lower pipe plate 1712. A heat exchange pipe 1703 passes through the corresponding holes of the upper pipe plate 1702 and the lower pipe plate 1712 to connect the upper pipe plate 1702 with the lower pipe plate 1712. Both ends of the heat exchange pipe 1703 are respectively welded on the upper pipe plate 1702 and the lower pipe plate 1712, so that, a closed cavity is formed among the upper pipe plate 1702, the lower pipe plate 1712, the outer side of the heat exchange pipe 1703 and the outer wall of the tank body 1711 and is communicated with the outside through the fresh air inlet flange 1706 and the hot air exhaust flange 1710. A plurality of pull rods 1705 are uniformly fixed to the lower pipe plate. A plurality of partition plates 1704 are uniformly arranged in a space between the fresh air inlet flange 1706 and the hot air exhaust flange 1710 in the tank body. The partition plates 1704 are fixed to the pull rods 1705. An inner cavity of the heat exchange pipe is communicated with the upper end cover 1701 and the lower end cover 1707. A U-shaped pipe 1713 is arranged at the bottom of a lower cover plate 1707.

The schematic diagram of the boiler system is shown in FIG. 25. The liquid high-temperature aerobic fermentation system 206 includes a liquid high-temperature aerobic fermentation reactor 206A. a liquid high-temperature aerobic fermentation reactor 206B and a liquid high-temperature aerobic fermentation reactor 206N, that is, includes totally N liquid high-temperature aerobic fermentation reactors (N≥1). The solid high-temperature aerobic fermentation system 207 includes a solid high-temperature aerobic fermentation, reactor 207A, a solid high-temperature aerobic fermentation reactor 207B and a solid high-temperature aerobic fermentation reactor 207M, that is, includes totally M solid high-temperature aerobic fermentation reactors (M≥1). A water inlet pipeline 503 of the boiler 512 is connected with the water outlet of a pressure water tank 501. A water inlet valve 502 is arranged on the water inlet pipeline 503. The water inlet of the pressure water tank 501 is connected with a water supplementing pipe 505. A water supplementing valve 501 is arranged on the water supplementing pipe 505. A water outlet pipeline 511 of the boiler 512 is connected to the input end of a three-way electric regulation valve 506. The two output ends of the three-way electric regulation valve 506 are respectively connected with the water inlet flanges of the plurality of liquid high-temperature aerobic fermentation reactors (206A, 206B and . . . 206N) and the water inlets of the solid high-temperature aerobic fermentation reactors (207A, 207R and . . . 207N) through the water outlet pipeline 511. The water outlet flange of each liquid high-temperature aerobic reactor 206 and the water outlet of each solid high-temperature aerobic fermentation reactor 207 are connected with a water return pipeline 508 of the boiler 512. Electromagnetic valves 507 are arranged on the water outlet pipelines of each liquid high-temperature aerobic reactor 206 and each solid high-temperature aerobic fermentation reactor 207. A temperature sensor is installed on the water outlet pipeline 511 of the boiler 512. A temperature sensor 507B, a circulating pump 513, an exhaust valve 509 and a pressure gauge 510 are also arranged on the water return pipeline 508 of the boiler 512.

### Implementation Mode I:

(1). The pigsty keeps off rainwater, and the rainwater is drained through the external drainage ditch 101 in time, thereby realizing rainwater and sewage separation. When pigs drink water, water leaking from the autodrinker for pigs and lips fall, into the U-shaped water collection cavity and then is drained into the external drainage ditch 101 through the water drainage pipeline in time, thereby realizing dunking water and sewage separation. Daily excrements (including feces and urine) of the pigs leak from the slatted floors 102 and fall onto the Y-shaped slope 105 or fall into the feces cleaning ditch 106. The feces and urine which fall onto the V-shaped slope 105 naturally slide into the feces cleaning ditch 106 under the gravity action. The feces scraping system is started regularly every day. The driving device 107 drives the feces scraper 108 through the driving rope 110 to move back and forth from the highest end to the lowest end along the bottom surface of the feces cleaning ditch 106. When the feces scraper 108 moves towards the feces collection pit 203, the limiting clip 108A clamps the scraper blade, and the scraper blade 108B drives the feces and urine to move forwards to finally collect the feces and urine into the feces collection pit 203. When the feces scraper 108 moves towards the other side of the feces collection pit 203, the restriction of the limiting clip 108A is released, and the scraper blade 108B is shoveled up by the driving rope 110, but the feces and urine may not move backwards. When the sensors 111 detect that the feces scraper 108 arrives at both ends of the feces cleaning ditch 106, the feces scraping control system 109 controls the feces scraping driving device 107 to stop the operation and then to operate oppositely after delay time. When there are pigs being slaughtered, the feces scraping system is started firstly to clean the feces and urine falling onto the Y-shaped slope 105 or falling into the feces cleaning ditch 106, and then the water is controlled to flush the pigsty. During pigsty flushing, the feces scraping system stops the operation, and the pigsty flushing water flows into the feces cleaning ditch 106 through the slatted floor 102 and finally flows into the pigsty flushing water pit 301.

(2) When the source separation pigsty 201 is flushed, the pigsty flushing water enters the pigsty flushing water pit 301 through the coarse grating and flows to the ABR 302 through the fine grating and the overflow pipe. The pigsty flushing water is subjected to sludge settling and anaerobic fermentation of the ABR 302, and then fermentation liquid enters the first SBR 303A. After the liquid level of the first SBR 303A reaches its designed liquid level, the test and control system 210 controls the electromagnetic valve 305A in front of the SBR 303 A to turn off the electromagnetic valve 305A in front of the first SBR 303A and turn on the electromagnetic valve 305B of the second SBR 303B, so as to allow the liquid levels of all the SBRs to reach the designed liquid levels respectively. The test and control system 210 realizes an aerobic-anaerobic alternating technical process on all SBRs (303A, 303B and 303X) according to an SBR technique by controlling intermittent aeration. After the SBRs (303A, 303B and . . . 303X) complete a complete SBR treatment technique, supernatant is conveyed to the ecological wetland 304 through the conveying equipment and then discharged. Filter residues produced by impurity removal through the gratings of the pigsty flushing water pit 301 and sludge produced by the ABR 302 and the SBRs 303 are conveyed into the solid high-temperature aerobic fermentation reactor 207 and are mixed with the feces for high-temperature aerobic fermentation treatment to prepare a solid organic fertilizer

(3). The feces and urine in a piglet pigsty are conveyed into the liquid high-temperature aerobic fermentation reactor 206 through the conveying equipment. A liquid part separated through the solid-liquid separation device 205 is conveyed into the liquid high-temperature aerobic fermentation reactor 206 through a conveying device, and a separated solid part is conveyed into the solid high-temperature aerobic fermentation reactor 207 through the conveying device.

(4) Auxiliary materials and high-temperature aerobic bacteria are conveyed into the solid high-temperature aerobic fermentation reactor 207 through conveying equipment. During feeding, the test and control system 210 starts all the power driving devices at the same time to allow all the power supporting wheel groups 701 to rotate at the same time to drive the horizontal drum 814 of the solid high-temperature aerobic fermentation reactor 207 to rotate forwards. By virtue of the action of the spiral stirring and anti-sticking device 809 in the solid high-temperature aerobic fermentation reactor 207, fermentation raw materials are conveyed to the discharge side, and organic wastes are shoveled up and fall down so as to be fully stirred and mixed with oxygen, thereby enlarging the contact area of the fermentation raw materials and the oxygen.

(5). The dead pigs and the placentas 1604 are put into the hanging basket through a forklift truck or other sets of transferring equipment. The lifting device 1405 lifts the hanging basket into the liquid high-temperature aerobic fermentation reactor 206 to immerse the whole hanging basket into the liquid, and at the same time, a proper amount of a composite microbial fermentation agent is inoculated into the liquid high-temperature aerobic fermentation reactor 206.

(6). The circulating pump 513 and the boiler 512 are started in sequence. Hot water enters the heat exchange coil of the liquid high-temperature aerobic fermentation reactor 206 and the jack of the solid high-temperature aerobic fermentation reactor 207 to respectively heat the materials in the liquid high-temperature aerobic fermentation reactor 206 and the solid high-temperature aerobic fermentation reactor 207. The boiler system 208 and the odor and flue gas treatment system 209 are started at the same time. Odor exhausted by the solid high-temperature aerobic fermentation reactor 207 is cooled by the odor heat exchange condenser A, then is conveyed to the biological deodorization. filtering tower 403A through the induced draft fan 402A for absorption and conversion and is discharged into the atmosphere through the exhaust port of the biological deodorization filtering tower 403A after reaching the standard. Hot air heated by the odor heat exchange condenser A is introduced into the solid high-temperature aerobic fermentation reactor 207 through the induced draft fan 402A to heat and supply oxygen to the material in the solid high-temperature aerobic fermentation reactor 207. The aeration fan 404 adjusts the opening of the three-way electric regulation valve 407 according to an oxygen demand of the material in the liquid high-temperature aerobic fermentation reactor 206 to aerate the liquid high-temperature aerobic fermentation reactor 206, so that the air input ends of the hearth of the boiler 512 and the three-way electric regulation valve 407 are in negative pressure states all the time, and flue gas generated by the boiler 512 and partial fresh air are mixed through the three-way electric regulation valve 407 and enter the liquid high-temperature aerobic fermentation reactor 206 tor aeration of the feces and urine liquid. Odor exhausted by the liquid high-temperature aerobic fermentation reactor 206 is cooled by the odor heat exchange condenser B, then is conveyed to the biological deodorization filtering tower 403B through the induced draft fan 402B for absorption and conversion and is discharged to the atmosphere through the exhaust port of the biological deodorization filtering tower 403B after reaching the standard. Hot air heated by the odor heat exchange condenser B is introduced into the hot water boiler 512 through the air blower of the hot water boiler 512 to provide fresh hot air for the hot water boiler 512. Condensed water produced by heat exchange between hot odor and cold air is drained into a natural ditch through the U-shaped pipes 1713 of the odor heat exchange condensers A and B.

(7) When detecting that the odor temperature detected by the temperature sensors (406A and 406B) installed on the main-path air inlet pipelines of the biological deodorization filtering towers (403A and 403B) is more than 40° C., the test and control system 210 turns on the electromagnetic valves (405A and 405C) on the air inlet pipelines of the odor heat exchange condensers (A and B) and turns off the electromagnetic valves (405B and 405D) of the bypass branches to allow the odor entering the deodorization filtering tower (403A and 403B) to be cooled by the odor heat exchange condensers (A and B). When detecting that the odor temperature defected by the temperature sensors ( 406A. and 406B) installed on the main-path air inlet pipelines of the biological deodorization filtering towers (403A and 403B) is less than 15° C. the test and control system 210 turns off the electromagnetic valves (405A and 405C) on the air inlet pipelines of the odor heat exchange condensers (A and B) and turns on the electromagnetic valves (405B and 405D) of the bypass branches to forbid the odor to enter the odor heat exchange condensers (A and B) for cooling, so that the biological deodorization filtering towers (403A and 403B) work in a temperature range between 15° C. and 40° C. so as to guarantee the deodorization effect and prevent dormancy or death of microorganisms in the biological deodorization filtering towers (403A and 403B).

(8) In an aerobic fermentation reaction process, the test and control system 210 controls the power driving device of the solid high-temperature aerobic fermentation reactor 207 to operate in a periodic intermittent operation manner of backward rotation-stop-backward rotation-stop . . . according to a detected temperature of the fermentation raw material or a set time. During rotating of the drum 814, under the driving of the contact blocks 1001, the shoveling plates of the stirring and anti-sticking device drive 809 drive the materials at the bottom of the horizontal drum 814 to move upwards along the inner wall of the drum 814, and the materials are separated from the shoveling plates and thrown away and fall back to the bottom of the horizontal drum 814 under the gravity action, so as to achieve stirring and air contact effects. By virtue of the action of the spiral shoveling plates in the solid high-temperature aerobic fermentation reactor 207, the backward rotating drum shovels up the materials and conveys the fermentation raw materials to the feed side, so that the fermentation materials may not compact the discharge side sealing cover 822. Because the cage-shaped structures of the stirring and anti-sticking device 809 collide with different contact blocks 1001 in the drum 814 and rotate under the driving of the contact blocks 1001, the cage-shaped structures and the inner wall of the drum 814 may slide relatively, so that the fermentation raw materials may not be adhered to the inner wall of the drum 814 of the solid high-temperature aerobic fermentation reactor 207, and the energy consumption caused by stirring and heat conduction is reduced to the minimum.

(9). The solid materials in the solid high-temperature aerobic fermentation reactor 207 are continuously fermented at more than 60° C. for more than 24 hours to complete the whole high-temperature aerobic fermentation process to prepare the solid organic fertilizer. The test and control system 210 controls to turn off the electromagnetic valves 507 at the front ends of the power supporting wheel group 701 of the solid high-temperature aerobic fermentation reactor 207 and the water inlet pipeline of the water jacket 811 and controls to bun on the discharge gate 820 at the same time. Then the test and control system 210 controls the power supporting wheel group 701 to continuously rotate forwards to discharge part of old fermented materials to the next working procedure for treatment through external conveying equipment.

(10). The material in the liquid high-temperature aerobic fermentation reactor 206 is continuously fermented at 60° C. or higher for more than 3 days to complete the whole high-temperature aerobic fermentation process. Hot fermentation liquid subjected to the high-temperature aerobic fermentation is immediately conveyed to the biogas generating pit 204 subjected to heat presentation treatment through a pipeline for high-temperature or medium-temperature anaerobic fermentation. The fermentation liquid is continuously anaerobically fermented at 35-60° C. for 15 to 20 days to complete the anaerobic fermentation process. After being diluted secondary fermentation liquid may be directly applied lot agriculture, and produced biogas may be applied to the boiler system 208 or power generation. Residues produced by the dead pigs 1604 are rotten, and hairs and bone residues are conveyed to the boiler 512 for incineration. Ash produced by incineration is conveyed to the solid high-temperature aerobic fermentation reactor 207 and is mixed with solid feces for fermentation, so as to prepare the solid organic fertilizer.

(11). The test and control system 210 is used for monitoring and acquiring key data of all aspects of the comprehensive treatment system and coordinately controlling all constituents of the comprehensive treatment system according to the acquired data:

\hat{1} in the high-temperature aerobic fermentation reaction process, the test and control system 210 automatically controls the opening of a circulating water three-way electric regulation valve 506 according to the temperatures of the materials in the high-temperature aerobic fermentation reactors (the liquid high-temperature aerobic fermentation reactor 206 and the solid high-temperature aerobic fermentation reactor 207), so that the temperatures of the fermentation materials are stabilized at a set temperature all the time when the temperature of the material in the high-temperature aerobic fermentation reactor (the liquid high-temperature aerobic fermentation reactor 206 or the solid high-temperature aerobic fermentation reactor 207) of a first fermentation object is less than the set value, the opening of the three-way electric regulation valve 506 in tins loop is 100%, and the openings in the loops of other high-temperature aerobic fermentation reactors (the liquid high-temperature aerobic fermentation reactor 207) or the solid high-temperature aerobic fermentation reactor 207) are 0; when the temperature of the material of the high-temperature aerobic fermentation reactor (the liquid high-temperature aerobic fermentation reactor 206 or the solid high-temperature aerobic fermentation reactor 207) of the first fermentation object is close to the set value, the test and control system 210 controls to turn on the electromagnetic valve in the loop of the second high-temperature aerobic fermentation reactor (the liquid high-temperature aerobic fermentation reactor 206 or the solid high-temperature aerobic fermentation reactor 207), and the three-way electric regulation valve 506 performs PID regulation to allow part of the hot circulating water to flow through the second high-temperature aerobic fermentation reactor, so that the second high-temperature aerobic fermentation reactor is heated under the condition of stabilizing the temperature of the material of the first high-temperature aerobic fermentation reactor at the set value, because the aerobic fermentation process is a heat release process, with the fermentation, the temperature of the material in the high-temperature aerobic fermentation reactor (the liquid high-temperature aerobic fermentation reactor 206 or the solid high-temperature aerobic fermentation reactor 207) continuously use up; when the temperature of the material in the high-temperature aerobic fermentation reactor of the first fermentation object is greater than the set value, the test and control system 210 slows down oi shuts off the heating of the boiler 512; under the action of the circulating pump 513, the circulating water is mixed with circulating water of the high-temperature aerobic fermentation reactors of the first fermentation object and the second fermentation object, resulting in that the temperature of the material in the high-temperature aerobic fermentation reactor of the first fermentation object is reduced, and the temperature of the material in the high-temperature aerobic fermentation reactor of the second fermentation object is increased; the three-way electric regulation valve 506 and the electromagnetic valve 507 are coordinately controlled by the test and control system 210 to convey fermentation reaction heat of the previous high-temperature aerobic fermentation reactor and heat generated by heating of the boiler to the second or Mth solid high-temperature aerobic fermentation reactor (207A, 207B and . . . 207M) or the Nth liquid high-temperature aerobic fermentation reactor (206A, 206B and . . . 206N), so that the temperatures of the materials in the high-temperature aerobic fermentation reactors (the liquid high-temperature aerobic fermentation reactor 206 or the solid high-temperature aerobic fermentation reactor 207) may be stabilized at the set value, and the heat energy generated by the fermentation reaction may be recycled; and

\hat{2} the test and control system 210 uploads the key data in a data region in the test and control system 210 to a cloud or remote server 211 for storage and backup through communication with a data gateway, so that all data of a treatment process evidence chain are stored for later inquiry, and service staff of a remote head office can find out faults and alarm of equipment operation by virtue of the data of the cloud and handle with the faults and alarm in time; and the data stored in the cloud are also favorable for completion and upgrading of the treatment system.

### Implementation Mode II:

(1). The pigsty keeps off rainwater and the rainwater is drained through the external drainage ditch 101 in time, thereby realizing rainwater and sewage separation When pigs drink water, water leaking from the autodrinker for pigs and lips fall into the U-shaped water collection cavity and then is drained into the external drainage ditch 101 through the water drainage pipeline in time, thereby realizing drinking water and sewage separation Daily excrements (including feces and urine) of the pigs leak from the slatted floors 102 and fall onto the V-shaped slope 105 or fall into the feces cleaning ditch 106. The feces and urine which fall onto the V-shaped slope 105 naturally slide into the feces cleaning ditch 106 under the gravity action. The feces scraping system is started regularly every day. The driving device 107 drives the feces scraper 108 through the driving rope 110 to move back and forth from the highest end to the lowest end along the bottom surface of the feces cleaning ditch 106. When the feces scraper 108 moves towards the feces collection pit 203, the limiting clip 108A clamps the scraper blade, and the scraper blade 108B drives the feces and urine to move forwards to finally collect the feces and urine into the feces collection pit 203. When the feces scraper 108 moves towards the other side of the feces collection pit 203, the restriction of the limiting clip 108A is released, and the scraper blade 108B is shoveled up by the driving rope 110, but the feces and urine may not move backwards. When the sensors 111 detect that the feces scraper 108 arrives at both ends of the feces cleaning ditch 106, the feces scraping control system 109 controls the feces scraping driving device 107 to stop the operation and then to operate oppositely after delay time. When there are pigs being slaughtered, the feces scraping system is started firstly to clean the feces and urine falling onto the V-shaped slope 105 or falling into the feces cleaning ditch 106, and then the water is controlled to flush the pigsty. During pigsty flushing the feces scraping system stops the operation, and the pigsty flushing water flows into the feces cleaning ditch 106 through the slatted floor 102 and finally flows into the pigsty flushing water pit 301.

(2) When the source separation pigsty 201 is flushed, the pigsty flushing water enters the pigsty flushing water pit 301 through the coarse grating and flows to the ABR 302 through the fine grating and the overflow pipe. The pigsty flushing water is subjected to sludge settling and anaerobic fermentation of the ABR 302, and then fermentation liquid enters the first SBR 303A. After the liquid level of the first SBR 303A reaches its designed liquid level, the test and control system 210 controls the electromagnetic valve 305A in front of the SBR 303A to turn off the electromagnetic valve 305A in front of the first SBR 303A and turn on the electromagnetic valve 305B of the second SBR 303B, so as to allow the liquid levels of all the SBRs to reach the designed liquid levels respectively. The test and control system 210 realizes an aerobic-anaerobic alternating technical process on all SBRs (303A, 303B and . . . 303X) according to an SBR technique by controlling intermittent aeration. After the SBRs (303A, 303B and . . . 303X) complete a complete SBR treatment technique, supernatant is conveyed to the ecological wetland 304 through the conveying equipment and then discharged. Filter residues produced by impurity removal through the gratings of the pigsty flushing water pit 301 and sludge produced by the ABR 302 and the SBRs 303 are conveyed into the solid high-temperature aerobic fermentation reactor 207 and are mixed with the feces for high-temperature aerobic fermentation treatment to prepare a solid organic fertilizer.

(3). The feces and urine in a piglet pigsty are conveyed into the liquid high-temperature aerobic fermentation reactor 206 through the conveying equipment. A liquid part separated through the solid-liquid separation device 205 is conveyed into the liquid high-temperature aerobic fermentation reactor 200 through a conveying device, and a separated solid part is conveyed into the solid high-temperature aerobic fermentation reactor 207 through the conveying device.

(4) Auxiliary materials and high-temperature aerobic bacteria are conveyed into the solid high-temperature aerobic fermentation reactor 207 through conveying equipment. During feeding, the test and control system 210 starts all the power driving devices at the same time to allow all the power supposing wheel groups 701 to rotate at the same time to drive the horizontal drum 814 of the solid high-temperature aerobic fermentation reactor 207 to rotate forwards. By virtue of the action of the spiral stirring and anti-sticking device 809 in the solid high-temperature aerobic fermentation reactor 207, fermentation raw materials are conveyed to the discharge side, and organic wastes are shoveled up and fall down so as to be fully stirred and mixed with oxygen, thereby enlarging the contact area of the fermentation raw materials and the oxygen.

(5). The dead pigs 1604 are conveyed into the combustion hearth of the boiler 512 through a forklift truck or other sets of transferring equipment. At the same time, a proper amount of a composite microbial fermentation agent is inoculated into the liquid high-temperature aerobic fermentation reactor 206.

(6). The circulating pump 513 and the boiler 512 are started in sequence. Hot water enters the heat exchange coil of the liquid high-temperature aerobic fermentation reactor 206 and the jack of the solid high-temperature aerobic fermentation reactor 207 to respectively heat the materials in the liquid high-temperature aerobic fermentation reactor 206 and the solid high-temperature aerobic fermentation reactor 207. The boiler system 208 and the odor and flue gas treatment system 209 are started at the same time. Odor exhausted by the solid high-temperature aerobic fermentation reactor 207 is cooled by the odor heat exchange condenser A, then is conveyed to the biological deodorization filtering tower 403A through the induced draft fan 402A for absorption and conversion and is discharged into the atmosphere through the exhaust port of the biological deodorization filtering tower 403A after reaching the standard. Hot air heated by the odor heat exchange condenser A is introduced into the solid high-temperature aerobic fermentation reactor 207 through the induced draft fan 402A to heat and supply oxygen to the material in the solid high-temperature aerobic fermentation reactor 207. Odor exhausted by the liquid high-temperature aerobic fermentation reactor 206 is cooled by the odor heat exchange condenser C, then is conveyed to the biological deodorization filtering tower 403B through the induced draft fan 402B for absorption and conversion and is discharged to the atmosphere through the exhaust port of the biological deodorization filtering tower 403B after reaching the standard. Hot air heated by the odor heat exchange condenser C is blown and pressurized by the aeration fan 404 and conveyed into the liquid high-temperature aerobic fermentation reactor 206. The feces and urine liquid is heated and aerated. Flue gas generated by the hot water boiler 512 is subjected to heat exchange and is cooled by the flue gas heat exchange condenser 601, is introduced into the biological deodorization filtering tower 403C through the induced draft fan 602 for absorption and conversion and is discharged to the atmosphere through the exhaust port of the biological deodorization filtering tower 403C after reaching the standard. Hot air heated by the flue gas heat exchange condenser 601 is blasted into the hearth of the hot water boiler 512 through the an blower of the hot water boiler 512 to provide fresh not air for the combustion of the hot water boiler 512. Condensed water produced by heat exchange between the odor heat exchange condensers A and C and the flue gas heat exchange condenser 601 is drained into a natural ditch through the U-shaped pipes 1713 of the odor heat exchange condensers A and C and the flue gas heat exchange condenser 601.

(7) When detecting that the odor temperature detected by the temperature sensors (406A and 406B) installed on the main-path air inlet pipelines of the biological deodorization filtering towers (403A and 403B) is more than 40° C., the test and control system 210 turns on the electromagnetic valves (405A and 405C) on the air inlet pipelines of the odor heat exchange condensers and turns off the electromagnetic valves (405B and 405D) of the bypass branches to allow the odor entering the deodorization filtering tower (403A and 403B) to be cooled by the odor heat exchange condensers (A, B and C). When detecting that the odor temperature detected by the temperature sensors (406A and 406B) installed on the main-path air inlet pipelines of the biological deodorization filtering towers (403A and 403B) is less than 15° C., the test and control system 210 turns off the electromagnetic valves (405A and 405C) on the air inlet pipelines of the odor heat exchange condensers (A, B and C) and turns on the electromagnetic valves (405B and 405D) of the bypass branches to forbid the odor to enter the odor heat exchange condensers (A, B and C) for cooling, so that the biological deodorization filtering towers (403A and 403B) work in a temperature range between 15° C. and 40° C., so as to guarantee the deodorization effect and prevent dormancy or death of microorganisms in the biological deodorization filtering towers (403A and 403B).

(8) In an aerobic fermentation reaction process, the test and control system 210 controls the power driving device of the solid high-temperature aerobic fermentation reactor 207 to operate in a periodic intermittent operation manner of backward rotation-stop-backward rotation-stop . . . according to a detected temperature of the fermentation raw material or a set time. During rotating of the drum 814, under the driving of the contact blocks 1001, the shoveling plates of the stirring and anti-sticking device drive 809 drive the materials at the bottom of the horizontal drum 814 to move upwards along the inner wall of the drum 814. and the materials are separated from the shoveling plates and thrown away and fall back to the bottom of the horizontal drum 814 under the gravity action, so as to achieve stirring and air contact effects. By virtue of the action of the spiral, shoveling plates in the solid high-temperature aerobic fermentation reactor 207, the backward rotating drum shovels up the materials and conveys the fermentation raw materials to the feed side, so that the fermentation materials may not compact the discharge side sealing cover 822. Because the cage-shaped structures of the stilling and anti-sticking device 809 collide with different contact blocks 1001 in the drum 814 and rotate under the driving of the contact blocks 1001, the cage-shaped structures and the inner wall of the drum 814 may slide relatively, so that the fermentation raw materials may not be adhered to the inner wall of the drum 814 of the solid high-temperature aerobic fermentation reactor 207, and the energy consumption caused by stirring and heat conduction is reduced to the minimum.

(9). The solid materials in the solid high-temperature aerobic fermentation reactor 207 are continuously fermented at more than 60° C. for more than 24 hours to complete the whole high-temperature aerobic fermentation process to prepare the solid organic fertilizer The test and control system 210 controls to turn off the electromagnetic valves 507 at the front ends of the power supporting wheel group 701 of the solid high-temperature aerobic fermentation reactor 207 and the water inlet pipeline of the water jacket 811 and controls to turn on the discharge gate 820 at the same time. Then the test and control system 210 controls the power supporting wheel group 701 to continuously rotate forwards to discharge part of old fermented materials to the next working procedure for treatment through external conveying equipment.

(10). The material in the liquid high-temperature aerobic fermentation reactor 206 is continuously fermented at 60° C. or higher tor more than 3 days to complete the whole high-temperature aerobic fermentation process. Hot fermentation liquid subjected to the high-temperature aerobic fermentation is immediately conveyed to the biogas generating pit 204 subjected to heat preservation treatment through a pipeline for high-temperature or medium-temperature anaerobic fermentation. The fermentation liquid is continuously anaerobically fermented at 35-60° C. for 15 to 20 days to complete the anaerobic fermentation process. After being diluted, secondary fermentation liquid may be directly applied for agriculture, and produced biogas may be applied to the boiler system 208 or power generation.

(11). The test and control system 210 is used for monitoring and acquiring key data of all aspects of the comprehensive treatment system and coordinately controlling all constituents of the comprehensive treatment system according to the acquired data:

\hat{1} in the high-temperature aerobic fermentation reaction process, the test and control system 210 automatically controls the opening of a circulating water three-way electric regulation valve 506 according to the temperatures of the materials in the high-temperature aerobic fermentation reactors (the liquid high-temperature aerobic fermentation reactor 206 and the solid high-temperature aerobic fermentation reactor 207), so that the temperatures of the fermentation materials are stabilized at a set temperature all the time: when the temperature of the material in the high-temperature aerobic fermentation reactor (the liquid high-temperature aerobic fermentation reactor 200 or the solid high-temperature aerobic fermentation reactor 207) of a first fermentation object is less than the set value, the opening of the three-way electric regulation valve 506 in this loop is 100%, and the openings in the loops of other high-temperature aerobic fermentation reactors (the liquid high-temperature aerobic fermentation reactor 206 or the solid high-temperature aerobic fermentation reactor 207) are 0; when the temperature of the material of the high-temperature aerobic fermentation reactor (the liquid high-temperature aerobic fermentation reactor 206 or the solid high-temperature aerobic fermentation reactor 207) of the first fermentation object is close to the set value, the test and control system 210 controls to turn on the electromagnetic valve in the loop of the second high-temperature aerobic fermentation reactor (the liquid high-temperature aerobic fermentation reactor 206 or the solid high-temperature aerobic fermentation reactor 207), and the three-way electric regulation valve 506 performs PID regulation to allow part of the hot circulating water to flow through the second high-temperature aerobic fermentation reactor, so that the second high-temperature aerobic fermentation reactor is heated under the condition of stabilizing the temperature of the material of the first high-temperature aerobic fermentation reactor at the set value; because the aerobic fermentation process is a heat release process, with the fermentation, the temperature of the material in the high-temperature aerobic fermentation reactor (the liquid high-temperature aerobic fermentation reactor 206 or the solid high-temperature aerobic fermentation reactor 207) continuously rise up- when the temperature of the material in the high-temperature aerobic fermentation reactor of the first fermentation object is greater than the set value, the test and control system 210 slows down or shuts off the heating of the boiler 512; under the action of the circulating pump 51 the circulating water is mixed with circulating water of the high-temperature aerobic fermentation reactor s of the first fermentation object and the second fermentation object, resulting in that the temperature of the material in the high-temperature aerobic fermentation reactor of the first fermentation object is reduced, and the temperature of the material in the high-temperature aerobic fermentation reactor of the second fermentation object is increased; the three-way electric regulation valve 506 and the electromagnetic valve 507 are coordinately controlled by the test and control system 210 to convey fermentation reaction heat of the previous high-temperature aerobic fermentation reactor and heat generated by heating of the boiler to the second or Mth solid high-temperature aerobic fermentation reactor (207A, 207B and . . . 207M) or file Nth liquid high-temperature aerobic fermentation reactor (206A, 206B and . . . 206N), so that the temperatures of the materials in the high-temperature aerobic fermentation reactors (the liquid high-temperature aerobic fermentation reactor 206 or the solid high-temperature aerobic fermentation reactor 207) may be stabilized at the set value, and the heat energy generated by the fermentation reaction may be recycled; and

\hat{2} the test and control system 210 uploads the key data in a data region in the test and control system 210 to a cloud or remote server 211 for storage and backup through communication with a data gateway, so that all data of a treatment process evidence chain are stored for later inquiry, and service staff of a remote head office can find out faults and alarm of equipment operation by virtue of the data of the cloud and handle with the faults and alarm in time and the data stored in the cloud are also favorable for completion and upgrading of the treatment system

### Implementation Mode III:

(1). The pigsty keeps off rainwater, and the rainwater is drained through the external drainage ditch 101 in time, thereby realizing rainwater and sewage separation. When pigs drink water, water leaking from the autodrinker for pigs and lips fall into the U-shaped water collection cavity and then is drained into the external drainage ditch 101 through the water drainage pipeline in time, thereby realizing drinking water and sewage separation. Daily excrements (including feces and urine) of the pigs leak from the slatted floors 102 and fall onto the Y-shaped slope 105 or fall into the feces cleaning ditch 106. The feces and urine which fall onto the V-shaped slope 108 naturally slide into the feces cleaning ditch 106 under the gravity action. The feces scraping system is started regularly every day. The driving device 107 drives the feces scraper 108 through the driving rope 110 to move back and forth from the highest end to the lowest end along the bottom surface of the feces cleaning ditch 106. When the feces scraper 108 moves towards the feces collection pit 203, the limiting clip 108A clamps the scraper blade, and the scraper blade 108B drives the feces and urine to move forwards to finally collect the feces and urine into the feces collection pit 203. When the feces scraper 108 moves towards the other side of the feces collection pit 203, the restriction of the limiting clip 108A is released, and the scraper blade 108B is shoveled up by the driving rope 110, but the feces and urine may not move backwards. When the sensors 111 detect that the feces scraper 108 arrives at both ends of the feces cleaning ditch 106, the feces scraping control system 109 controls the feces scraping driving device 107 to stop the operation and then to operate oppositely after delay time. When there are pigs being slaughtered, the feces scraping control system 109 is started firstly to clean the feces and urine falling onto the V-shaped slope 105 or falling into the feces cleaning ditch 106, and then the water is controlled to flush the pigsty. During pigsty flushing, the feces scraping control system 109 stops the operation, and the pigsty flushing water flows into the feces cleaning ditch 106 through the slatted floor 102 and finally flows into the pigsty flushing water pit 301.

(2) When the source separation pigsty 201 is flushed, the pigsty flushing water enters the pigsty flushing water pit 301 through the coarse grating and flows to the ABR 302 through the fine grating and the overflow pipe. The pigsty flushing water is subjected to sludge settling and anaerobic fermentation of the ABR 302, and then fermentation liquid enters the first SBR 303A. After the liquid level of the first SBR 303A reaches its designed liquid level, the test and control system 210 controls the electromagnetic valve 305 A in front of the SBR 303A to turn off the electromagnetic valve 305A in front of the first SBR 303A and turn on the electromagnetic valve 305B of the second SBR 303B, so as to allow the liquid levels of all the SBRs to reach the designed liquid levels respectively. The test and control system 210 realizes an aerobic-anaerobic alternating technical process on all SBRs (303A, 303B and . . . 303X) according to an SBR technique by controlling intermittent aeration. After the SBRs (303A, 303B and . . . 303X) complete a complete SBR treatment technique, supernatant is conveyed to the ecological wetland 304 through the conveying equipment and then discharged. Filter residues produced by impurity removal through the gratings of the pigsty flushing water pit 301 and sludge produced by the ABR 302 and the SBRs 303 are conveyed into the solid high-temperature aerobic fermentation reactor 207 and are mixed with the feces for high-temperature aerobic fermentation treatment to prepare a solid organic fertilizer.

(3). The feces and urine in a piglet pigsty are conveyed into the liquid high-temperature aerobic fermentation reactor 206 through the sludge pump 112 and a connection pipeline thereof. A liquid part separated through the solid-liquid separation device 205 is conveyed into the liquid high-temperature aerobic fermentation reactor 206 through a conveying device, and a separated solid part is conveyed into the solid high-temperature aerobic fermentation reactor 207 through the conveying device.

(4) Auxiliary materials and high-temperature aerobic bacteria are conveyed into the solid high-temperature aerobic fermentation reactor 207 through conveying equipment. During feeding, the test and control system 210 starts all the power driving devices at the same tune to allow all the power supporting wheel groups 701 to rotate at the same time to drive the horizontal drum 814 of the solid high-temperature aerobic fermentation reactor 207 to rotate forwards. By virtue of the action of the spiral stirring and anti-sticking device 809 in the solid high-temperature aerobic fermentation reactor 207, fermentation raw materials are conveyed to the discharge side, and organic wastes are shoveled up and fall down so as to be fully stirred and mixed with oxygen, whereby enlarging the contact area of the fermentation raw materials and the oxygen.

(5). The dead pigs 1604 are conveyed into the combustion hearth of the boiler 512 through a forklift truck or other sets of transferring equipment. At the same time, a proper amount of a composite microbial fermentation agent is inoculated into the liquid high-temperature aerobic fermentation reactor 206.

(6). The circulating pump 513 and the boiler 512 are started in sequence Hot water enters the heat exchange coil of the liquid high-temperature aerobic fermentation reactor 206 and the jack of the solid high-temperature aerobic fermentation reactor 207 to respectively heat the materials in the liquid high-temperature aerobic fermentation reactor 206 and the solid high-temperature aerobic fermentation reactor 207. The boiler system 208 and the odor and flue gas treatment system 209 are started at the same time. Odor exhausted by the solid high-temperature aerobic fermentation reactor 207 is cooled by the odor heat exchange condenser A, then is conveyed to the biological deodorization filtering tower 403A through the induced draft fan 402A for absorption and conversion and is discharged into the atmosphere through the exhaust port of the biological deodorization filtering tower 403A after reaching the standard. Hot air heated by the odor heat exchange condenser A is introduced into the solid high-temperature aerobic fermentation reactor 207 through the induced draft fan 402A to heat and supply oxygen to the material in the solid high-temperature aerobic fermentation reactor 207. Odor exhausted by the liquid high-temperature aerobic fermentation reactor 206 is cooled by the odor heat exchange condenser C, then is conveyed to the biological deodorization filtering tower 403B through the induced draft fan 402B for absorption and conversion and is discharged to the atmosphere through the exhaust port of the biological deodorization filtering tower 403B after reaching the standard. Hot air heated by the odor heat exchange condenser C is blown and pressurized by the aeration fan 404 and conveyed into the liquid high-temperature aerobic fermentation reactor 206. The feces and urine liquid is heated and aerated. Flue gas generated b the hot water boiler 512 is subjected to heat exchange and is cooled by the flue gas heat exchange condenser 601, is introduced into the biological deodorization filtering tower 403C through the induced, draft fan 602 for absorption and conversion and is discharged to the atmosphere through the exhaust port of the biological deodorization filtering tower 403C after reaching the standard. Hot air heated by the flue gas heat exchange condenser 601 is blasted into the hearth of the hot water boiler 512 through the air blower of the hot water boiler 512 to provide fresh hot air for the combustion of the hot water boiler 512. Condensed water produced by heat exchange between the odor hear exchange condensers A and C and the flue gas heat exchange condenser 601 is drained into a natural ditch through the U-shaped pipes 1713 of the odor heat exchange condensers A and C and the flue gas heat exchange condenser 601.

(7) When detecting that the odor temperature detected by the temperature sensors (406A and 406B) installed on the main-path air inlet pipelines of the biological deodorization filtering towers (403A and 403B) is more than 40° C., the test and control system 210 turns on the electromagnetic valves (405A and 405C) on the air inlet pipelines of the odor heat exchange condensers and turns off the electromagnetic valves (405B and 405D) of the bypass branches to allow the odor entering the deodorization filtering tower (403A and 403B) to be cooled by the odor heat exchange condensers A and C. When detecting that the odor temperature detected by the temperature sensors (406A and 406B) installed on the main-path air inlet pipelines of the biological deodorization filtering towers (403A and 403B) is less than 15° C. the test and control system 210 turns off the electromagnetic valves (405A and 405C) on the air inlet pipelines of the odor heat exchange condensers A and C and turns on the electromagnetic valves (405B and 405D) of the bypass branches to forbid the odor to enter the odor heat exchange condensers A and C for cooling, so that the biological deodorization filtering towers (403A and 403B) work in a temperature range between 15° C. and 40° C., so as to guarantee the deodorization effect and prevent dormancy or death of microorganisms in the biological deodorization filtering towers (403A and 403B).

(8) In an aerobic fermentation reaction process, the test and control system 210 controls the power driving device of the solid high-temperature aerobic fermentation reactor 207 to operate in a periodic intermittent operation manner of backward rotation-stop-backward rotation-stop . . . according to a detected temperature of the fermentation raw material or a set time. During rotating of the drum 814, under the driving of the contact blocks 1001, the shoveling plates of the stirring and anti-sticking device drive 809 drive the materials at the bottom of the horizontal drum 814 to move upwards along the inner wall of the drum 814, and the materials are separated from the shoveling plates and thrown away and fall back to the bottom of the horizontal drum 814 under the gravity action, so as to achieve stirring and air contact effects. By virtue of the action of the spiral shoveling plates in the solid high-temperature aerobic fermentation reactor 207, the backward rotating drum shovels up the materials and conveys the fermentation raw materials to the feed side, so that the fermentation materials may not compact the discharge side sealing cover 822. Because the cage-shaped structures of the stirring and anti-sticking device 809 collide with different contact blocks 1001 in the drum 814 and rotate under the driving of the contact blocks 1001, the cage-shaped structures and the inner wall of the drum 814 may slide relatively, so that the fermentation raw materials may not be adhered to the inner wall of the drum 814 of the solid high-temperature aerobic fermentation reactor 207, and the energy consumption caused by stirring and heat conduction is reduced to the minimum.

(9). The solid materials in the solid high-temperature aerobic fermentation reactor 207 are continuously fermented at more than 60° C. for more than 24 hours to complete the whole high-temperature aerobic fermentation process to prepare the solid organic fertilizer. The test and control system 210 controls to turn off the electromagnetic valves 507 at the front ends of the power supporting wheel group 701 of the solid high-temperature aerobic fermentation reactor 207 and the water inlet pipeline of the water jacket 811 and controls to turn on the discharge gate 820 at the same time. Then the test and control system 210 controls the power supporting wheel group 701 to continuously rotate forwards to discharge part of old fermented materials to the next working procedure for treatment through external conveying equipment.

(10). The material in the liquid high-temperature aerobic fermentation reactor 206 is continuously fermented at 60° C. or higher for more than 3 days to complete the whole high-temperature aerobic fermentation process. After being diluted, the fermentation liquid may be directly applied for agriculture.

(11). The test and control system 210 is used for monitoring and acquiring key data of all aspects of the comprehensive treatment system and coordinately controlling all constituents of the comprehensive treatment system according to the acquired data:

\hat{1} in the high-temperature aerobic fermentation reaction process, the test and control system 210 automatically controls the opening of a circulating water three-way electric regulation valve 506 according to the temperatures of the materials in the high-temperature aerobic fermentation reactors (the liquid high-temperature aerobic fermentation reactor 206 and the solid high-temperature aerobic fermentation reactor 207), so that the temperatures of the fermentation materials are stabilized at a set temperature all the time: when the temperature of the material in the high-temperature aerobic fermentation reactor (the liquid high-temperature aerobic fermentation reactor 206 or the solid high-temperature aerobic fermentation reactor 207) of a first fermentation object is less than the set value, the opening of the three-way electric regulation valve 506 in this loop is 100% and the openings in the loops of other high-temperature aerobic fermentation reactors (the liquid high-temperature aerobic fermentation reactor 206 or the solid high-temperature aerobic fermentation reactor 207) are 0; when the temperature of the material of the high-temperature aerobic fermentation reactor (the liquid high-temperature aerobic fermentation reactor 200 or the solid high-temperature aerobic fermentation reactor 207) of the first fermentation object is close to the set value, the test and control system 210 controls to turn on the electromagnetic valve in the loop of the second high-temperature aerobic fermentation reactor (the liquid high-temperature aerobic fermentation reactor 206 or the solid high-temperature aerobic fermentation reactor 207), and the three-way electric regulation valve 506 performs PID regulation to allow part of the hot circulating water to flow through the second high-temperature aerobic fermentation reactor, so that the second high-temperature aerobic fermentation reactor is heated under the condition of stabilizing the temperature of the material of the first high-temperature aerobic fermentation reactor at the set value; because the aerobic fermentation process is a heat release process, with the fermentation, the temperature of the material in the high-temperature aerobic fermentation reactor (the liquid high-temperature aerobic fermentation reactor 206 or the solid high-temperature aerobic fermentation reactor 207) continuously rise up; when the temperature of the material in the high-temperature aerobic fermentation reactor of the first fermentation object is greater than the set value, the test and control system 210 slows down or shuts off the heating of the boiler 512, under the action of the circulating pump 513, the circulating water is mixed with circulating water of the high-temperature aerobic fermentation reactors of the first fermentation object and the second fermentation object, resulting in that the temperature of the material in the high-temperature aerobic fermentation reactor of the first fermentation object is reduced, and the temperature of the material in the high-temperature aerobic fermentation reactor of the second fermentation object is increased; the three-way electric regulation valve 506 and the electromagnetic valve 507 are coordinately controlled by the test and control system 210 to convey fermentation reaction heat of the previous high-temperature aerobic fermentation reactor and heat generated by heating of the boiler to the second or Mth solid high-temperature aerobic fermentation reactor (207A, 207B and . . . 207M) or the Nth liquid high-temperature aerobic fermentation reactor (206A, 206B and . . . 206N), so that the temperatures of the materials in the high-temperature aerobic fermentation reactors (the liquid high-temperature aerobic fermentation reactor 206 or the solid high-temperature aerobic fermentation reactor 207) may be stabilized at the set value, and the heat energy generated by the fermentation reaction may be recycled; and

\hat{2} the test and control system 210 uploads the key data in a data region in the test and control system 210 to a cloud or remote server 211 for storage and backup through communication with a data gateway, so that all data of a treatment process evidence chain are stored for later inquiry, and service staff of a remote head office can find out faults and alarm of equipment operation by virtue of the data of the cloud 211 and handle with the faults and alarm in time; and the data stored in the cloud 211 are also favorable for completion and upgrading of the treatment system.

We claim:

1. A system for comprehensive treatment of cultivation pollution in a scalable pig farm comprising a source separation pigsty a pigsty flushing water treatment system, a solid-liquid separation system, a solid high-temperature aerobic fermentation system, a liquid high-temperature aerobic fermentation system, an odor and flue gas treatment system, a boiler system and a test and control system, wherein the source separation pigsty is designed into a pigsty that separates feces and urine from rainwater, pigsty flushing water and residual drinking water; the rainwater and the residual drinking water are discharged into an outdoor drainage ditch; the feces and urine are conveyed to a feces collection pit of the solid-liquid separation system; the pigsty flushing water is discharged into a pigsty flushing water pit of the pigsty flushing water treatment system; the solid-liquid separation system is composed of a sludge pump installed at a bottom of the feces collection pit, a solid-liquid separation device and conveying equipment; the sludge pump pumps the feces and urine to the solid-liquid separation device; solids separated by the solid-liquid separation device are conveyed to a feed port of the solid high-temperature aerobic fermentation system, and liquid separated by the solid-liquid separation device is conveyed to a liquid inlet of the liquid high-temperature aerobic fermentation system; fermentation odor exhaust ports of the solid high-temperature aerobic fermentation system and the liquid high-temperature aerobic fermentation system and a flue gas exhaust port of the boiler system are connected with the odor and flue gas treatment system through pipelines; the boiler system comprises a boiler, a circulating pump, a hot water pipeline and a water return pipeline; the hot water pipeline of the boiler is connected with a heat exchange jacket or coil pipe of the solid high-temperature aerobic fermentation system and a jacket or coil pipe of the liquid high-temperature aerobic fermentation system; the circulating pump is installed in the water return pipeline; all sensors of the test and control system are disposed in the above systems to detect all key parameters; and the rest and control system controls connection of the above systems.

2. The system according to claim 1, wherein the pigsty flushing water treatment system is composed of the pigsty flushing water pit, an ABR (anaerobic baffled reactor) and a plurality of parallel-connected SBRs (sequencing batch reactors); a water overflow port is formed in an upper part of the pigsty flushing water pit; a large grating and a small grating are respectively installed on a water inlet outer side and a water outlet inner side of the pigsty flushing water pit; the water outlet side of the overflow port is connected with a water inlet of the ABR through an overflow pipe, a water outlet of the ABR is connected with water inlets of the parallel-connected SBRs through pipelines respectively; an electromagnetic valve is installed in front of the water inlet of each SBR; the water outlet of each SBR is connected to an ecological wetland through a pipeline; and settled sludge in the pigsty flushing water pit, in the ABR and in the SBRs are conveyed to a feed port of the solid high-temperature aerobic fermentation reactor and then are mixed with feces for fermentation to prepare a solid organic fertilizer

3. The system according to claim 1, wherein the solid high-temperature aerobic fermentation system comprises 1 to X solid high-temper a true aerobic fermentation reactors; X is more than or equal to 1, each solid high-temperature aerobic fermentation reactor is composed of an inclined horizontal drum, a teed side sealing cover labyrinth sealing device, a discharge side sealing cover labyrinth sealing device; a power supporting wheel group, a stirring and anti-sticking device and an integrated base; a water jacket is arranged outside the horizontal drum; a feed side is higher than a discharge side; the horizontal drum, a feed side sealing cover, a discharge side sealing cover and the labyrinth sealing devices on both sides form a closed fermentation space; a feed hole and an exhaust hole are formed in an upper part of the feed side scaling cover; an air inlet hole is formed in an upper part of the discharge side sealing cover; a discharge hole is formed in a lower part of the discharge side sealing cover; a discharge gate is installed on a discharge hole; the stirring and anti-sticking device is positioned in the horizontal drum which is disposed on the power supporting wheel group; and the power supporting wheel group, the feed side sealing cover and the discharge side sealing cover are fixed to the inclined integrated base to form a whole.

4. The system according to claim 3, wherein a structure and principle of the feed side sealing cover labyrinth sealing device are completely the same as those of the discharge side sealing cover labyrinth sealing device, and each of the structures is that two or more concentric cylindrical hoods having unequal diameters are perpendicularly welded and fixed to an inner side of the sealing cover, and the cylindrical hoods are consistent in height; correspondingly, a radial lining ring is welded and fixed to an inner wall of the drum at an end part of the horizontal drum; periphery of the lining ring is hermetically welded and fixed with the inner wall of the drum; one, two or more concentric cylindrical bodies having unequal diameters are perpendicularly welded on the lining ring, and the concentric cylindrical bodies are consistent in height, moreover, the height of each of the concentric cylindrical bodies is equal to that of each of the concentric cylindrical hoods on the sealing cover; and the concentric cylindrical hoods on the inner sides of the sealing covers and the concentric cylindrical bodies on the lining ring at the end part of the horizontal drum are alternately sheathed and sealed in a labyrinth manner.

5. The system according to claim 4, wherein according to a length of the horizontal drum, the stirring and anti-sticking device is composed of one or more cage-shaped structures; when the horizontal drum is relatively short, the stirring and anti-sticking device is composed of only one cage-shaped structure; when the horizontal drum is relatively long, the stirring and anti-sticking device is composed of a plurality of cage-shaped structures; each of the cage-shaped structures is composed of two coaxial supporting plates and a plurality of shoveling plates; the supporting plates are circular rings, and both ends of each of the plurality of shoveling plates are respectively connected and fixed with the two coaxial supporting plates; correspondingly, contact blocks are disposed on the inner wall of the horizontal drum; the plurality of shoveling plates are parallel to axes of the cage-shaped structures, or the plurality of shoveling plates and the axes of the cage-shaped structures form inclined angles, or the plurality of shoveling plates are of spiral curve shapes; and when the horizontal drum rotates, the contact blocks on the inner wall drive the stirring and anti-sticking device to rotate.

6. The system according to claim 1, wherein the liquid high-temperature aerobic fermentation system is composed of 1 to N liquid high-temperature aerobic fermentation reactors; N is more than or equal to 1; each liquid high-temperature aerobic fermentation reactor mainly comprises a top cover, a tank body, a lifting device and a hanging basket; a feed port is formed in the top cover, and a discharge port is formed in a bottom of the tank body; a heat exchange coil is arranged in the tank and is connected with an external hot water pipeline through a water inlet flange and a water outlet flange which are installed on the tank body; an aeration device is also arranged in the tank and is connected with an aeration pipeline and a fan through an air inlet flange installed on the tank body; an exhaust port flange is also installed on the top cover of the tank body to exhaust aeration waste gas; the discharge port is connected to a biogas generating pit through a pipeline; the aeration device is connected to an external fan through an air inlet pipeline; the lifting device is used for lifting and transferring the top cover component and the hanging basket, the hanging basket is composed of a hanging basket main body, a hanging basket door and a lock catch; the hanging basket door is laterally opened from one side and is used for allowing putting dead livestock or placentas into the hanging basket; steel meshes are welded at an upper part, a bottom and a side wall of the hanging basket main body; and the hanging basket carries the dead livestock and the placentas and puts the dead livestock and the placentas into the feces and urine fermentation liquid for fermentation.

7. The system according to claim 1, wherein the odor and flue gas treatment system comprises an odor heat exchange condenser, a flue gas heat exchange condenser, a biological deionization filtering tower, an induced draft fan, a temperature sensor, a three-way electric regulation valve and an electromagnetic valve; each heat exchange condenser comprises an upper end cover, a tank body and a lower end cover which are connected and fixed in sequence; an upper end of the upper end cover is provided with an odor inlet flange; an odor collection pipeline is connected and fixed with the odor inlet flange, a lower part of the side wall of the tank body is provided with a fresh air inlet flange, and a upper part is provided with a hot air exhaust flange; an upper pipe plate is installed at an upper part of the tank body and a lower pipe plate is installed at a lower part of the tank body, a plurality of holes are uniformly formed in the upper and the lower pipe plates; a heat exchange pipe passes through corresponding holes of the upper pipe plate and the lower pipe plate to connect the upper pipe plate with the lower pipe plate; both ends of the heat exchange pipe are respectively fixed to the upper and the lower pipe plates, so that a closed cavity is formed among the ripper and the lower pipe plates, an outer side of the heat exchange pipe and an outer wall of the tank body and is communicated with outside through the fresh air inlet flange and the hot air exhaust flange; a plurality of pull rods are uniformly fixed to the lower pipe plate; a plurality of partition plates are uniformly arranged in a space between the fresh air inlet flange and the hot air exhaust flange in the tank body; the partition plates are fixed to the pull rods; an inner cavity of the heat exchange pipe is communicated with the upper end cover and the lower end cover: a U-shaped pipe is arranged at a bottom of a lower cover plate; an odor exhaust flange is arranged on a side wall of the lower end cover; a volume of the lower end cover of each heat exchange condenser is greater than or equal to that of the upper end cover; each partition plate is of a trimmed circular structure and has a diameter equal to an inner diameter of the tank body; the partition plates are uniformly distributed in the tank body in a disordered manner along an axial direction; and the partition plates are fixed to the pull rods, so that fresh air flows in a “Z”-shaped manner.

8. The system according to claim 7, wherein the odor and flue gas treatment system is configured to realize the following treatment systems:
(1) an odor treatment system of the solid high-temperature aerobic fermentation system, wherein the exhaust port of the solid high-temperature aerobic fermentation reactor is connected with a heat exchange air inlet of the odor heat exchange condenser A; a heat exchange exhaust port of the odor heat exchange condenser A is connected with an input end of the induced draft fan; an electromagnetic valve and a bypass branch are arranged on an air inlet pipeline of the odor heat exchange condenser A; the bypass branch of the odor heat exchange condenser A is provided with an electromagnetic valve; an output end of the induced draft fan is connected with an air inlet of the biological deodorization filtering tower, and a temperature sensor is installed on a main-path air inlet pipeline of the biological deodorization filtering tower; a biological deodorization filler is arranged in the biological deodorization filtering tower; the air inlet of the odor heat exchange condenser A is connected with atmosphere, and an air output port connected to an air inlet of the solid high-temperature aerobic fermentation reactor through a pipeline; and
(2) an odor treatment system of the high-temperature aerobic fermentation system and a flue gas treatment system of the boiler system, wherein a flue gas exhaust port of the boiler is connected with one air inlet input end of a three-way electric regulation valve, and the other air inlet input end of the three-way electric regulation valve is connected with atmosphere; an output end of the three-way electric regulation valve is connected with an input end of an aeration fan; an output end of the aeration fan is connected with an air inlet flange of the liquid high-temperature aerobic fermentation reactor; an exhaust flange of the liquid high-temperature aerobic fermentation reactor is connected with a heat exchange air inlet of an odor heat exchange condenser B; a heat exchange exhaust port of the odor heat exchange condenser B is connected with the input end of the induced draft fan; an electromagnetic valve and a bypass branch are arranged on an air inlet pipeline of the odor heat exchange condenser B; a bypass branch of the odor heat exchange condenser B is provided with an electromagnetic valve; the output end of the induced draft fan is connected with an air inlet of a biological deodorization filtering tower, and a temperature sensor is installed on a main-path air inlet pipeline of the biological deodorization filtering tower; a biological deodorization filler is arranged in the biological deodorization filtering tower; an air inlet of the odor heat exchange condenser B is connected with the atmosphere, and an air output port is connected to an air inlet of the boiler through a pipeline.

9. The system according to claim 7, wherein when the boiler is used for incinerating objects, such as garbage and dead pigs which, block the aeration heads easily, the odor and flue gas treatment system adopts the following connection modes that:
(1) in an odor treatment system of the solid high-temperature aerobic fermentation system, the exhaust, port of the solid high-temperature aerobic fermentation reactor is connected with a heat exchange air inlet of an odor heat exchange condenser A; a heat exchange exhaust port of the odor heat exchange condenser A is connected with an input end of the induced draft fan; an electromagnetic valve and a bypass branch are arranged on an air inlet pipeline of the odor heat exchange condenser A; the bypass branch of the odor heat exchange condenser A is provided with the electromagnetic valve; an output end of the induced draft fan is connected with an air inlet of a biological deodorization filtering tower, and a temperature sensor is installed on a main-path air inlet pipeline of the biological deodorization filtering tower; a biological deodorization filler is arranged in the biological deodorization filtering tower, the air inlet of the odor heat exchange condenser A is connected with atmosphere, and an air output port is connected to an air inlet of the solid high-temperature aerobic fermentation reactor through a pipeline;
(2) in an odor treatment system of the liquid high-temperature aerobic fermentation system, an exhaust flange of a liquid high-temperature aerobic fermentation reactor is connected with a heat exchange air inlet of an odor heat exchange condenser C; a heat exchange exhaust port of the odor heat exchange condenser C is connected with the input end of the induced draft fan; an electromagnetic valve and a bypass branch are arranged on an air inlet pipeline of the odor heat exchange condenser C; the bypass branch of the odor heat exchange condenser C is provided with, the electromagnetic valve; the output end of the induced draft fan is connected with the air inlet of the biological deodorization filtering tower, and the temperature sensor is installed on the main-path air inlet pipeline of the biological deodorization filtering tower; the biological deodorization filler is arranged in the biological deodorization filtering tower; the air inlet of the odor heat exchange condenser C is connected with atmosphere; an air output port is connected to an air inlet of an aeration fan through a pipeline; and an air outlet of the aeration fan is connected with an air inlet of the liquid high-temperature aerobic fermentation reactor; and
(3) in a flue gas treatment system of the boiler system, a flue gas exhaust port of the boiler is connected with a heat exchange air inlet of the flue gas heat exchange condenser; a heat exchange exhaust port of the flue gas heat exchange condenser is connected wish the input end of the induced draft fan; the output end of the induced draft fan is connected with the air inlet of the biological deodorization filtering tower; the biological deodorization filler is arranged in the biological deodorization filtering tower; the air inlet of the fine gas heat exchange condenser is connected with atmosphere; and an air output port is connected to an air inlet of the boiler through a pipeline.

10. The system according to claim 1, wherein the boiler system mainly comprises the boiler, the circulating pump, a pressure water tank, a three-way electric regulation valve and an electromagnetic valve, a water outlet pipeline of the boiler is connected to an input end of the three-way electric regulation valve; two output ends of the three-way electric regulation valve are respectively connected with water inlet flanges of parallel-connected liquid high-temperature aerobic fermentation reactors and parallel-connected solid high-temperature aerobic fermentation reactors through the water outlet pipeline; water outlet pipelines of the liquid high-temperature aerobic fermentation reactors and the solid high-temperature aerobic fermentation reactors are connected with the electromagnetic valve, a water outlet of the electromagnetic valve is connected with the water return pipeline of the boiler; temperature sensors me respectively arranged on the water outlet pipeline and the water return pipeline of the boiler; and the circulating pump is further installed on the water return pipeline to allow circulating water to form a loop.

11. The system according to claim 1, wherein the test and control system comprises a sensor, a controller and a data gateway which are installed in system equipment; the controller acquires key data of all aspects of the system equipment through the sensor and coordinately controls all parts of the system equipment according to the acquired data, the controller further communicates with the data gateway; and the controller sends the key data to a cloud or remote server through the data gateway for later inquiry and management.

12. A method for comprehensive treatment of system according to claim 1, comprising that:
(I) a source separation pigsty separates rainwater from sewage, separates drinking water from sewage and separates feces and urine from pigsty flushing water; rainwater and residual drinking water of pigs are discharged to an external ditch; pigsty flushing water is conveyed into a pigsty flushing water pit; feces and urine are conveyed into a feces collection pit;
(II) when a liquid level of the pigsty flushing water in the pigsty flushing water pit reaches an overflow port, the pigsty flushing water is filtered through gratings; filtrate flows into ABRs (anaerobic baffled reactors) through an overflow pipeline; a test and control system controls to turn on or turn off electromagnetic valves in front of SBRs (sequencing batch reactors) to allow liquid treated by the ABRs to respectively flow into different SBRs, and the SBRs are aerated intermittently according to an SBR technique to realize an aerobic-anaerobic technological process; before a complete SBR technique cycle is completed, the control system turns off the electromagnetic valve in front of the reactor and turns on the electromagnetic valve in front of a next SBR; when the SBRs complete the complete SBR treatment technique, a water pump pumps supernatant into an ecological wetland for discharging; sludge in the pigsty flushing water pit, the ABRs and the SBRs are regularly conveyed to a feed port of a solid high-temperature aerobic fermentation reactor and mixed with feces for fermentation to prepare a solid organic fertilizer;
(III) the feces and urine of piglets in a suckling period and a nursing period are conveyed into a liquid high-temperature aerobic fermentation reactor, namely a solid part separated by a solid-liquid separation device from the feces and urine of fattening pigs, boars and sows is conveyed into the solid high-temperature aerobic fermentation reactor, and a liquid part separated through solid-liquid separation is conveyed into the liquid high-temperature aerobic fermentation reactor;
(IV) auxiliary materials and high-temperature aerobic bacteria are conveyed into the solid high-temperature aerobic fermentation reactor through conveying equipment; during feeding, the test and control system starts all power driving devices at the same time to allow all power supporting wheel groups to rotate at the same nine to drive a horizontal drum of the solid high-temperature aerobic fermentation reactor to rotate forwards; by action of a spiral stirring and anti-sticking device in the solid high-temperature aerobic fermentation reactor, fermentation raw materials are conveyed to a discharge side, and organic waste is shoveled up and dropped down so that organic waste is fully stirred and mixed with oxygen, thereby enlarging a contact area of fermentation raw materials and the oxygen;
(V) dead pigs and placentas are put into a hanging basket through a forklift truck or other sets of transferring equipment; a lifting device lifts the hanging basket into the liquid high-temperature aerobic fermentation reactor to immerse the whole hanging basket into the liquid, and at the same time, a proper amount of a composite microbial fermentation agent is inoculated into the liquid high-temperature aerobic fermentation reactor for high-temperature aerobic fermentation; if certain pig farms are qualified for treating the dead pigs and the placentas in an incineration way or other sanitary ways, the hanging basket is omitted;
(VI) a circulating pump and a boiler are started in sequence; hot water enters a jack of the solid high-temperature aerobic fermentation reactor and a heat exchange coil of the liquid high-temperature aerobic fermentation reactor to respectively heat solids in the solid high-temperature aerobic fermentation reactor and liquid in the liquid high-temperature aerobic fermentation reactor.
(VII) a boiler system and an odor and flue gas treatment system are started at the same time, and the odor and fine gas treatment system is configured to realize the following methods:
(1) an odor treatment method of a solid high-temperature aerobic fermentation system odor exhausted by the solid high-temperature aerobic fermentation reactor through an exhaust port is cooled by an odor heat exchange condenses A, then is absorbed and converted through a biological deodorization filtering tower and is discharged after meeting a standard; hot air subjected to heat exchange through the odor heat exchange condenser A enters the solid high-temperature aerobic fermentation reactor through an air inlet of the solid high-temperature aerobic fermentation reactor to provide fresh hot air for the solid high-temperature aerobic fermentation reactor; and
(2) an odor treatment method of a high-temperature aerobic fermentation system and a flue gas treatment method of the boiler system: an aeration fan adjusts an opening of a three-way electric regulation valve according to air oxygen demand of a material in the liquid high-temperature aerobic fermentation reactor to aerate the liquid high-temperature aerobic fermentation reactor, so that air input ends of a hearth of the boiler and the three-way electric regulation valve are in negative pressure states all the time, and flue gas generated by the boiler and partial fresh air are mixed through the three-way electric regulation valve and enter the liquid high-temperature aerobic fermentation reactor for aeration; odor exhausted by the liquid high-temperature aerobic fermentation reactor through the exhaust port is cooled by an odor heat exchange condenser B, then is absorbed and converted through the biological deodorization filtering tower and is discharged after reaching the standard; and hot air subjected to hear exchange of the odor heat exchange condenser B enters the hearth of the boiler to provide fresh hot air for the boiler;
(VIII) when the boiler is used for incinerating objects, such as garbage and dead pigs which block the aeration heads easily, the odor and flue gas treatment system is configured to realize the following methods.
(1) an odor treatment method of the solid high-temperature aerobic fermentation system, the odor exhausted by the solid high-temperature aerobic fermentation reactor through the exhaust port is cooled by the odor heat exchange condenser A, then is absorbed and converted through the biological deodorization filtering tower and is discharged after reaching the standard; hot air subjected to heat exchange through the odor heat exchange condenser A enters the solid high-temperature aerobic fermentation reactor through the air inlet of the solid high-temperature aerobic fermentation reactor to provide fresh hot air for the solid high-temperature aerobic fermentation reactor;
(2) an odor treatment method of the liquid high-temperature aerobic fermentation system: the odor exhausted by the liquid high-temperature aerobic fermentation reactor through the exhaust port is cooled by the odor heat exchange condenser C, then is absorbed and converted through the biological deodorization filtering tower and is discharged after reaching the standard; hot air subjected to heat exchange of the odor heat exchange condenser C is blasted into the liquid high-temperature aerobic fermentation reactor through the aeration fan to provide fresh hot air for the liquid high-temperature aerobic fermentation reactor; and
(3) flue gas exhausted by the boiler is cooled by the flue gas heat exchange condenser, then is absorbed and converted through the biological deodorization filtering tower and is discharged after reaching the standard; and cold air is heated by the flue gas heat exchange condenser and then enters the hearth of the hot water boiler to provide fresh hot air for the hot water boiler;
(IX) when detecting that the odor temperature detected by a temperature sensor installed on a main-path air inlet pipeline of the biological deodorization filtering tower is more than 40° C., the test and control system turns on the electromagnetic valves on the air inlet pipelines of the odor heat exchange condensers A, B and C and turns off the electromagnetic valves of the bypass branches to allow the odor entering the biological deodorization filtering tower to be cooled by the odor heat exchange condensers A, B and C at first; when detecting that odor temperature detected by the temperature sensor installed on the main-path air inlet pipeline of the biological deodorization filtering tower is less than 15° C., the test and control system turns off the electromagnetic valves on the air inlet pipelines of the odor heat exchange condensers A, B and C and turns on the electromagnetic valves of the bypass branches to forbid the odor to enter the odor heat exchange condensers A, B and C for cooling, so that the biological deodorization filtering tower works in a temperature range between 15° C. and 40° C., so as to guarantee the deodorization effect and prevent dormancy and death of microorganisms in the biological deodorization filtering tower;
(X) when hot odor and cold air are subjected to heat exchange in the odor heat exchange condensers A, B and C, and hot flue gas and cold air are subjected to heat exchange in the flue gas heat exchange condenser, produced condensed water is discharged by the odor heat exchange condensers A, B and C and the flue gas heat exchange condenser and then is drained to an external ditch through a pipeline;
(XI) in an aerobic fermentation reaction process, the test and control system controls a power driving device of the solid high-temperature aerobic fermentation reactor to operate in a periodic intermittent operation manner of “backward rotation-stop-backward rotation-stop . . . ” according to a detected temperature of the fermentation raw material or a set time; during rotation of a drum, under driving of contact blocks welded on an inner wall of a horizontal drum, shoveling plates of a stirring and anti-sticking device drive materials at an inner bottom of the horizontal drum to move upwards along an inner wall of the drum, and materials are separated from the shoveling plates and thrown away and fall back to the bottom of the horizontal drum under gravity action, so as to achieve stirring and air contact effects; by action of spiral shoveling plates in the solid high-temperature aerobic fermentation reactor, a backward rotating drum shovels up the materials and conveys the fermentation raw materials to a feed side, so that the fermentation materials are not compacted on a discharge side sealing cover; because cage-shaped structures of the stirring and anti-sticking device collide with different contact blocks in the drum and rotate under the driving of the contact blocks, the cage-shaped structures and the inner wall of the drum slide relatively, so that the fermentation raw materials are not adhered to the inner wall of the drum of the solid high-temperature aerobic fermentation reactor, and energy consumption caused by stirring and heat conduction is minimized;
(XII) solids in the solid high-temperature aerobic fermentation reactor are continuously fermented at 60° C. or higher for more than 24 hours to complete the whole high-temperature aerobic fermentation process to prepare the solid organic fertilizer; the test and control system controls to turn off electromagnetic valves at front ends of the power driving device and a water inlet pipeline of the water jacket and controls to turn on a discharge gate at the same time; then the test and control system controls the power driving device to continuously rotate forwards to discharge part of old fermentation materials to a next working procedure for treatment through external conveying equipment;
(XIII) the material in the liquid high-temperature aerobic fermentation reactor is continuously fermented at 60° C. or higher for more than 3 days to complete the whole high-temperature aerobic fermentation process; if carcasses of dead pigs and placentas are not placed, the feces and urine are continuously fermented at 60° C. or higher for 24 hours to complete high-temperature harmless treatment; further, a discharge port of the liquid high-temperature aerobic fermentation reactor is provided with a heat preservation anaerobic fermentation reactor (a biogas generating pit), hot fermentation liquid subjected to the high-temperature aerobic fermentation is immediately conveyed to the heat preservation anaerobic fermentation reactor subjected to heat preservation treatment through a pipeline for high-temperature or medium-temperature anaerobic fermentation: the fermentation liquid is continuously anaerobically fermented at 35-60° C. for 15 to 20 days to complete the anaerobic fermentation process; after being diluted, secondary fermentation liquid is directly applied for agriculture, and produced biogas is applied to the boiler system or power generation; residues produced by the dead pigs are rotten, and hairs and bone residues are conveyed to a furnace for incineration, ash produced by incineration is conveyed to the solid high-temperature aerobic fermentation reactor and is mixed with solid feces for fermentation, so as to prepare a solid organic fertilizer;
(XIV) the test and control system is used for monitoring and acquiring key data of all aspects of the system for comprehensive treatment and coordinately controlling all constituents of the system for comprehensive treatment according to the acquired data;
(1) in the high-temperature aerobic fermentation reaction process, the test and control system automatically controls the opening of a circulating water three-way electric regulation valve according to the temperatures of the materials in the high-temperature aerobic fermentation reactors, so that the temperatures of the fermentation materials are stabilized at a set temperature all the time: when the temperature of the material in the high-temperature aerobic fermentation reactor of a first fermentation object is less than the set value, the opening of the three-way electric regulation valve in this loop is 100%, and the openings of the loops of other high-temperature aerobic fermentation reactors are 0; when the temperature of the material of the first fermentation object is close to the set value, the test and control system controls to turn on the electromagnetic valve in the loop of the second high-temperature aerobic fermentation reactor, and the three-way electric regulation valve performs PID regulation to allow the hot circulating water part to flow through the second high-temperature aerobic fermentation reactor, so that the second high-temperature aerobic fermentation reactor is heated under the condition of stabilizing the temperature of the material of the first high-temperature aerobic fermentation reactor at the set value; because the aerobic fermentation process is a heat release process, with the fermentation, the temperatures of the materials in the high-temperature aerobic fermentation reactors continuously rise up; when the temperature of the material in the high-temperature aerobic fermentation reactor of the first fermentation object is greater than the set value, the test and control system slows down or shuts off the heating of the boiler, under the action of the circulating pump, the circulating water of the high-temperature aerobic fermentation reactors of the first fermentation object and the second fermentation object is mixed, resulting in that the temperature of the material in the high-temperature aerobic fermentation reactor of the first fermentation object is reduced, and the temperature of the material in the high-temperature aerobic fermentation reactor of the second fermentation object is increased; the three-way electric legislation valve and the electromagnetic valve are coordinately controlled by the test and control system to convey fermentation reaction heat of the previous high-temperature aerobic fermentation reactor and heat generated by heating of the boiler to the second or Mth solid high-temperature aerobic fermentation reactor or the Nth liquid high-temperature aerobic fermentation reactor, so that the temperatures of the materials in the high-temperature aerobic fermentation reactors may be stabilized at the set value, and the heat energy generated by the fermentation reaction may be recycled; and
(2) the test and control system uploads the key data in a data region in the test and control system to a cloud or remote server for storage and backup through communication with a data gateway, so that all data of a treatment process evidence chain are stored for later inquiry and service staff of a remote head office can find out faults and alarm of equipment operation by virtue of the cloud data and handle with the faults and alarm in time; and the data stored in the cloud are also favorable for completion and upgrading of the treatment system.

13. The method according to claim 12, wherein the odor and flue gas treatment system is configured to realize the following methods:
(1) an odor treatment method of the solid high-temperature aerobic fermentation system: odor exhausted by the solid high-temperature aerobic fermentation reactor through an exhaust port is cooled by an odor heat exchange condenser A, then is absorbed and converted through a biological deodorization filtering tower and is discharged after reaching the standard; hot air subjected to heat exchange through the odor heat exchange condenser A enters the solid high-temperature aerobic fermentation reactor through an air inlet of the solid high-temperature aerobic fermentation reactor to provide fresh hot air for the solid high-temperature aerobic fermentation reactor; and
(2) an odor treatment method of a high-temperature aerobic fermentation system and a flue gas treatment method of the boiler system; an aeration fan adjusts the opening of a three-way electric regulation valve according to an oxygen demand of a material in the liquid high-temperature aerobic fermentation reactor to aerate the liquid high-temperature aerobic fermentation reactor, so that the air input ends of a hearth of the boiler and the three-way electric regulation valve are in negative pressure states all the time, and flue gas generated by the boiler and partial fresh air are mixed through the three-way electric regulation valve and enter the liquid high-temperature aerobic fermentation reactor; odor exhausted by the liquid high-temperature aerobic fermentation reactor through the exhaust port is cooled by an odor heat exchange condenser B, then is absorbed and converted through the biological deodorization filtering tower and is discharged after reaching the standard; and hot air subjected to heat exchange of the odor heat exchange condenser B enters the hearth of the boiler to provide fresh hot air for the boiler.

14. The method according to claim 12, wherein the odor and flue gas treatment system is configured to realize the following methods:
(1) an odor treatment method of the solid high-temperature aerobic fermentation system: the odor exhausted by the solid high-temperature aerobic fermentation reactor through the exhaust port is cooled by the odor heat exchange condenser A, then is absorbed and converted through the biological deodorization filtering tower and is discharged after reaching the standard; hot air subjected to heat exchange through the odor heat exchange condenser A enters the solid high-temperature aerobic fermentation reactor through the air inlet of the solid high-temperature aerobic fermentation reactor to provide fresh hot air for the solid high-temperature aerobic fermentation reactor;
(2) an odor treatment method of the liquid high-temperature aerobic fermentation system: odor exhausted by the liquid high-temperature aerobic fermentation reactor through the exhaust port is cooled by the odor heat exchange condenser C, then is absorbed and converted through the biological deodorization filtering tower and is discharged after reaching the standard; hot air subjected to heat exchange of the odor heat exchange condenser C is blasted into the liquid high-temperature aerobic fermentation reactor through the aeration fan to provide fresh hot air for the liquid high-temperature aerobic fermentation reactor; and
(3) flue gas exhausted by the boiler is cooled by the flue gas heat exchange condenser, then is absorbed and converted through the biological deodorization filtering tower and is discharged after reaching the standard; and cold air is heated by the flue gas heat exchange condenser and then enters the hearth of the hot water boiler to provide fresh hot air for the hot water boiler.
